(19) United States
(12) Patent Application Publication
(10) Pub. No.: US 2006/0233750 A1
(43) Pub. Date: Oct. 19, 2006

(54) Materials and method of modulating the immune response

(76) Inventor: Jim Xiang, Saskatoon (CA)

Correspondence Address: BERESKIN AND PARR, 40 KING STREET WEST, BOX 401, TORONTO, ON, M5H 3Y2, CA

(21) Appl. No.: 11/401,220

(22) Filed: Apr. 11, 2006

Related U.S. Application Data
(60) Provisional application No. 60/671,465, filed on Apr. 15, 2005.

(30) Foreign Application Priority Data
Apr. 15, 2005 (CA) 2,504,279

(51) Int. Cl. A61K 38/20; A61K 39/395; C12N 5/08

(52) U.S. Cl. 424/85.2; 424/145.1; 435/372

(57) ABSTRACT

Methods and materials to modulate the immune response to treat or prevent a disease, including methods of making T helper-antigen presenting cells and methods of using these cells. The invention also relates to methods of making exosome-absorbed dendritic cells and the uses of these cells to modulate the immune response to treat or prevent a disease.

### FIELD OF THE INVENTION

The invention relates to a method of modulating the immune response to treat or prevent a disease. In particular, the method relates to a method of making T helper-antigen presenting cells, and to methods of using the T helper-antigen presenting cells to modulate the immune response to treat or prevent a disease. The invention also relates to methods of making exosome-absorbed dendritic cells and exosome-absorbed T helper cells, and the uses of these cells to modulate the immune response to treat or prevent a disease.

### BACKGROUND OF THE INVENTION

Generation of effective cytotoxic T lymphocyte (CTL) responses to minor histocompatibility or tumor antigens not associated with danger signals often requires help from CD4⁺ T helper (Th) cells via cross-priming (1). Such help was originally thought to be mediated by CD4⁺ T cell IL-2 acting at short range to promote CD8⁺ T cell proliferation (2).

Two models of CD4⁺ T help for CD8⁺ CTL responses have been proposed previously, including the passive model of three-cell interaction (3,4) and the dynamic model of sequential two-cell interactions by antigen presenting cells (APC) (5). The three-cell model suggested that activated CD4⁺ T cells and naïve CD8⁺ T cells must interact simultaneously with a common APC, and that the CD4⁺ Th cells provide CD8⁺ T cell help via expression of Interleukin 2 (IL-2) (FIG. 1A). The conundrum, however, is how a rare antigen-specific CD4⁺ Th cell and an equally rare antigen-specific CD8⁺ T cell (typically 1 in 10⁵-10⁶ T cells) would simultaneously find the same antigen peptide-carrying APC in an unprimed animal (6). Instead, Ridge et al (5) have proposed a dynamic model of two sequential interactions, in which an APC first offers co-stimulatory signals to a CD4⁺ Th cell and then to a CD8⁺ CTL cell (FIG. 1B). According to this model, the APC-stimulated CD4⁺ Th cells must first reciprocally counter-stimulate the APCs (through CD40 ligand signaling) such that this newly “conditioned” APC can then directly co-stimulate CD8⁺ CTLs. Support for this model comprises evidence that antigen-specific CTL responses can be induced by vaccination with either large numbers of APC activated in vitro through CD40 signaling or, in either major histocompatibility complex (MHC) class II knockout (KO) or CD4⁺ T cell-depleted mice, by high level activation of APCs in vivo with anti-CD40 Ab (5,7-9). Although this model provides a possible explanation for the conditional nature of T-cell help for CTL responses, the experimental conditions used in the above studies may well not accurately model the physiology of Th cell-dependent immune responses in vivo. In addition, a scarcity caveat remains (10), in that very small numbers of antigen-bearing APCs (11) must first activate and be conditioned by the rare naïve antigen-specific CD4⁺ Th cells, and then find and activate in turn equally rare naïve Ag-specific CD8⁺ CTL. In addition, this model does not explain how IL-2 from Th cells' would be precisely targeted to Ag-specific CD8⁺ Ag-specific CTLs. Furthermore, the life span of an activated dendritic cell (DC) in the T cell zone of a lymph node is around 48 hours (11-13), possibly due to CD4⁺ T cell killing of the cognate APCs (14-15), whereas the antigen-specific CTL response is first detected at around day 5 in the lymph nodes (11,16). Thus, this dynamic model also does not explain compellingly the temporal gap between antigen presentation and the acquisition of CTL effector function in vivo.

It is recognized that stimulation of T cells by APCs involves at least two signaling events: one elicited by TCR recognition of peptide-MHC complexes and the other by costimulatory molecule signaling (e.g., T cell CD28/APC CD80) (17). A consequence of such Ag-specific T cell-APC interactions is the formation an immunological synapse, comprising a central cluster of TCR-MHC-peptide complexes and CD28-CD80 interactions surrounded by rings of engaged accessory molecules (e.g., complexed LFA-1-CD54) (18,19). One important feature of synapse physiology is that APC-derived surface molecules are transferred to the Th cells during the course of their TCR internalization followed by recycling (20,21).

Dendritic cells process exogenous antigens in endosomal compartments such as multivesicular endosomes (22) which can fuse with plasma membrane, thereby releasing antigen presenting vesicles called “exosomes” (23-25). Exosomes are 50-90 nm diameter vesicles containing Ag presenting molecules (MHC class I, class II, CD1, hsp70-90) tetraspan molecules (CD9, CD63, CD81), adhesion molecules (CD11b, CD54) and CD86 costimulatory molecules (26-28).

### SUMMARY OF THE INVENTION

The present inventor has demonstrated that CD4⁺ T cells can acquire the synapse-composed MHC class II and costimulatory molecules (CD54 and CD80), and bystander MHC class I/peptide complexes from antigen presenting cells. In addition, the inventor has demonstrated that the molecules acquired by the CD4⁺ T cells are functional, and that these CD4⁺ T cells can act as CD4⁺ T helper-antigen presenting cells (Th-APC) to stimulate the immune system in vitro and in vivo, particularly the CTL response.

The inventor has also shown that exosomes derived from dendritic cells display MHC class I/peptide complexes, CD11c, CD40, CD54 and CD80.

In addition, the inventor has shown that exosomes derived from dendritic cells can be absorbed onto CD4⁺ T cells. These exosome-absorbed CD4⁺ T cells express antigen presenting machinery derived from the dendritic cell, including peptide/MHC complexes, and costimulatory CD54 and CD80 molecules. These exosome-absorbed CD4⁺ T cells can act as Th-APC to stimulate the immune system in vitro and in vivo, particularly the CTL response.

Also, the inventor has shown that the antigen presenting machinery and costimulatory molecules can be transferred from activated dendritic cells to CD4⁺ T cells, and that these T cells can act as Th-APC to stimulate the immune system in vitro and in vivo, particularly the CTL response.

Further, the inventor has shown that the exosomes derived from dendritic cells can be absorbed onto dendritic cells, particularly mature dendritic cells. These exosome-absorbed dendritic cells express high levels of peptide/MHC class I complexes and costimulatory CD40, CD54, and CD80 molecules. These exosome-absorbed dendritic cells are potent stimulators of the immune system in vitro and in vivo, particularly the CTL response.

Accordingly, the invention provides a method of making a T helper-antigen presenting cell comprising contacting an exosome derived from a dendritic cell with a CD4⁺ T cell under conditions that allow absorption of the exosome on the CD4⁺ T cell.

Also, the invention provides a method of making a T helper-antigen presenting cell comprising contacting a CD4⁺ T cell with an activated dendritic cell under conditions that allow for transfer of molecules from the dendritic cell to the CD4⁺ T cell.

The invention also includes the isolated T helper-antigen presenting cell made according to the methods of the invention.

In addition, the invention provides a method of enhancing the immune response to treat or prevent a disease comprising administering an effective amount of T helper-antigen presenting cell to an animal in need thereof. The present invention also provides a use of an effective amount of T helper-antigen presenting cells to treat or prevent a disease.

Further, the invention provides a pharmaceutical composition for preventing or treating a disease comprising an effective amount of T helper-antigen presenting cells and a pharmaceutically acceptable carrier, diluent or excipient.

The invention also includes methods of making exosome-absorbed dendritic cells comprising contacting an exosome derived from a first dendritic cell with a second dendritic cell under conditions that allow absorption of the exosome on the second dendritic cell. The invention also includes the isolated exosome-absorbed dendritic cell made according to the methods of the invention.

In addition, the invention includes methods of enhancing the immune response to treat or prevent a disease comprising administering an effective amount of an exosome-absorbed dendritic cell to an animal in need thereof.

Further, the invention includes pharmaceutical compositions for preventing or treating a disease comprising an effective amount of an exosome-absorbed dendritic cell and a pharmaceutically acceptable carrier, diluent or excipient.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows three models for the delivery of CD4⁺ T help to CD8⁺ CTL. (A) The “passive”, three-cell interaction model, in which APC simultaneously present Ag to the T helper and the CTL, but deliver co-stimulatory signals only to the helper. The CD4⁺ Th cell in turn produces IL-2, which is required for CTL activation. (B) The dynamic model of sequential two-cell interactions by APCs, in which the APC offers co-stimulatory signals to the CD4⁺ T helper, which reciprocally “licenses” the APC (left side of panel) such that it can only then directly co-stimulate the CTL (right side). (C) The new dynamic model of sequential two-cell interactions, in which APCs “license” CD4⁺ T helper cells to act as APCs (Th-APCs). APCs directly transfer MHC class I/Ag complexes and co-stimulatory molecules to expanding populations of IL-2-producing Th cells, which thereby act directly as Th1-APCs to simulate CTL activation.

FIG. 2 shows analysis of OVA expression by flow cytometry.

(a) EG7 (thick solid lines) and EL4 (thick dotted lines), and (b) BL6-10_{OVA} (thick solid lines) and BL6-10 (thick dotted lines) tumor cells were stained with the rabbit anti-OVA antibody (Sigma), followed with the FITC-goat anti-rabbit IgG antibody, and then analyzed by flow cytometry. Tumor cells stained with normal rabbit serum were employed as control populations (thin dotted lines). One representative experiment of two is displayed.

FIG. 3 shows transfer of DC membrane molecules to active CD4⁺ T cells. (A) CFSE-labeled DC_{OVA} were incubated with Con A-stimulated CD4⁺ T cells from OT II mice. T cells with (thick solid lines) and without (thick dotted lines) incubation of DC_{OVA} were stained with Abs and analyzed for expression of H-2 K^{b}, Ia^{b}, CD54 and CD80 by flow cytometry, respectively. (B) CFSE-labeled DC_{OVA} were incubated with Con A-stimulated CD4⁺ T cells from H-2 K^{b}, Ia^{b}, CD54 and CD80 gene KO OT II mice, respectively. T cells with (thick solid lines) and without (thick dotted lines) incubation of DC_{OVA} were stained with Abs and analyzed for expression of the above molecules, respectively. T cells with incubation of DC_{OVA} were also stained with isotype-matched Abs and employed as control populations (thin dotted lines). (C) DC_{OVA}-activated CD4⁺ T cells (Th-APCs) from OT II mice were stained with a panel of Abs (thick solid lines) and analyzed by flow cytometry. The control CD4⁺ T cells (thin dotted lines) were only stained with isotype-matched Abs. (D) DC_{OVA}-activated CD4⁺ T cells (Th-APCs) from H-2 K^{b}, Ia^{b, CD}54 and CD80 gene KO OT II mice, respectively, were stained with a panel of Abs (thick solid lines). The control CD4⁺ T cells (thin dotted lines) were only stained with isotype-matched Abs. One representative experiment of two in the above different experiments is shown.

FIG. 4 shows membrane acquisition analysis by confocal fluorescence microscopy. CFSE-labeled DC_{OVA} were incubated with Con A-stimulated CD4⁺ T cells from (A) H-2 K^{b}, (B) CD54 and (C) CD80 gene KO OT II mice, stained with fluorochrome-labeled Abs, and analyzed by confocal fluorescence microscopy. Images include DCs (larger cells) alone, T (smaller) cells alone or a mixture of DC and T cells (a) under differential interference contrast, (b) with a cell-surface stain consisting of ECD (red)-Ab for either H-2K^{b}, CD54, or CD80, (c) with cytoplasmic CFSE stain (green), and (d) with both stains. The data confirm that (i) DC_{OVA} (larger cells), but not gene-deleted T cells (smaller cells), express H-2 K^{b}, CD54, and CD80 molecules (arrows), and (ii) during co-culture of DC_{OVA} with T cells, the T cells acquire H-2 K^{b}, CD54, and CD80 molecules (arrow heads). One representative experiment of two is shown.

FIG. 5 shows in vivo membrane transfer assay. The CD4⁺ T cells purified from OT II/Ia^{b-/-} and OT II/CD80^{-/-} mice were transferred into wild-type C57BL/6 mice, respectively. The first group of mice remained untreated and the second group of mice were immunized with DC_{OVA}. The CD4⁺ OT II/Ia^{b-/-} and OT II/CD80^{-/-} T cells were then purified from the first (thick dotted lines) and the second group (solid lines) of mice and then stained with the FITC-anti-Ia^{b} and FITC-anti-CD80 antibodies and the FITC-conjugated isotype-matched antibodies (thin dotted lines) for flow cytometric analysis, respectively. One representative experiment of three is shown.

FIG. 6 shows that CD4⁺ T-APCs stimulate RF3370 and OT I CD8⁺ T cells. (A) MHC class I presentation of OVA to RF3370 hybridoma by Th-APCs. The amount of IL-2 secretions of stimulated RF3370 cells in examining wells were subtracted by the amounts of IL-2 in wells containing DC_{OVA}, Th-APC and Con A-OT II alone, respectively. *, p<0.01 (Student t test) versus cohorts of Con A-OT II. (B) In vitro CD8⁺ T cell proliferation assay. Varying numbers of irradiated Th-APCs, K^{b-/-} Th-APCs, Con A-OT II and DC_{OVA} cells were co-cultured with naïve OT I or B6 CD8⁺ T cells. After three days, the proliferative responses of the CD8⁺ T cells were determined by ³H-thymidine uptake assays. (C) Th-APCs were cultured with OT I CD8⁺ T cells either separated in transwells (transwell) or not (all other bars). In the latter cultures, the impact on OT I CD8⁺ T cell proliferation of adding each of the neutralizing reagents, all neutralizing reagents together (mixed reagents), or all control Abs and fusion proteins (control reagents) was assessed. In one set of wells, supernatants from cultured Th-APCs (supernate) were added to the CD8⁺ T cells in place of the Th-APCs themselves. *, p<0.01 (Student t test) versus cohorts of Th-APC. (D) In vivo CD8⁺ T cell proliferation assay. CFSE-labeled OT I CD8⁺ T cells were i.v. injected into C57BL/6 mice. Twelve hours later, each mouse was i.v. given Th-APCs or Con A-OT II cells or DC_{OVA} or PBS, then 3 days later the numbers of division cycles of the CFSE-labeled CD8⁺ T cells in the recipient spleens were determined by flow cytometry. One representative experiment of three in the above different experiments is shown.

FIG. 7 shows that CD4⁺ T-APC induce the development of antigen-specific CTL activity in vitro and in vivo. In vitro cytotoxicity assay. (A) Three types of activated CD8⁺ T cells (DC_{OVA}/OT I, Th-APC/OT I, and Con A-OT II/OT I) were used as effector (E) cells, whereas ⁵¹Cr-labeled EG7 or control EL-4 tumor cells used as target (T) cells. (B) Th-APCs were used as effector (E) cells, whereas 51 Cr-labeled EG7, DCs, DC_{OVA}, LB27 and EG7OVAII cells used as target (T) cells. The data are presented as the percent specific target cell lysis in ⁵¹Cr-release assay. Each point represents the mean of triplicate cultures. (C) In vivo cytotoxicity assay. C57BL/6 splenocytes differentially labeled to be CFSE^{high} and CFSE^{low}, were pulsed with OVAI and Mut1 peptide, respectively. These splenocytes were then i.v. injected at ratio of 1:1 into mice immunized with DC_{OVA}, Th-APCs or Con A-OT II cells, or PBS. Sixteen hours later, the CFSE^{high} or CFSE^{low} cells remaining in the spleens were determined by flow cytometry. The value in each panel represents the percentage of CFSE^{high} cells versus CFSE^{low} cells remaining in the spleens.

FIG. 8 shows immune protection of lung metastasis in mice immunized with Th-APCs. Pulmonary metastases were formed in different groups of immunized mice by intravenous injection of 0.5×10⁶ BL6-10_{OVA} or BL6-10 tumor cells. Four weeks later, mouse lungs were removed. The extent of lung metastasis in 6 different groups of mice described in Exp I of Table 1 was displayed.

FIG. 9 is a phenotypic analysis of DC and DC-derived exosomes by flow cytometry. Flow cytometric analysis of (a) dendritic cells and DC-derived exosomes, and (b) OT II CD4⁺ cells. DC and DC-derived exosomes as well as OT II CD4⁺ cells (thick solid lines) were stained with a panel of Abs and then analyzed by flow cytometry. These cells and exosomes were also stained with isotype-matched irrelevant Abs, respectively, and employed as control populations (thin dotted lines).

FIG. 10 shows exosome uptake by CD4⁺ T cells. (a) Both naïve and active OT II and C57BL/6 CD4⁺ T cells with (thick solid lines) and without (thin dotted lines) uptake of EXO_{CFSE} were analyzed for CFSE expression by flow cytometry. (b) In the blocking assay, active OT II CD4⁺ aT cells were treated with anti-Ia^{b}, anti-LFA-1, CTLA-4/Ig, a mixture of these reagents or a mixture of matched isotype Abs (as control) on ice for 30 min, respectively, and then incubated with EXO_{CFSE}. The fractions of CFSE positive T cells were analyzed after co-culture for 4 h at 37° C. (c, e) Both naïve and active OT II CD4⁺ T cells with (thick solid lines) and without (thick dotted lines) uptake of EXO_{OVA} were analyzed for expression of a panel of surface molecules including H-2 K^{b}, CD54, CD80 and pMHC I by flow cytometry. Irrelevant isotype-matched Abs was used as controls (thin dotted lines). (d, f) Both naïve and active OT II CD4⁺ cells from H-2 K^{b}, CD54 and CD80 gene knock out mice were also co-cultured with (thick solid lines) and without (thin dotted lines) EXO_{OVA}, and then analyzed for expression of H-2 K^{b}, CD54 and CD80 by flow cytometry, respectively. One representative experiment of two is displayed.

FIG. 11 shows stimulation of CD8⁺ memory T cell responses in vitro. (a) In vitro CD8⁺ cell proliferation assay. EXO_{OVA} (10 μg/ml), DC_{OVA}, nT_{EXO}, aT_{EXO} and Con A-activated OTII T (aT) cells and their 2-fold dilutions were co-cultured with a constant number of OT I CD8⁺ T cells in presence or absence of CD4⁺25⁺ Tr cells. After three days, the proliferation response of CD8⁺ T cells was determined by ³H-thymidine uptake assay. (b) The impact of aT_{EXO} stimulation of OT I CD8⁺ T cell proliferation by adding each of the neutralizing reagents, a mixture of neutralizing reagents (mixed reagents), and a mixture of control Abs and fusion proteins (control reagents) was assessed. *, p<0.05 versus cohorts without adding any neutralizing reagent (Student's t test). (c) Phenotypic analysis of in vitro aT_{EXO}-primed CD8⁺ T cells. CFSE-labeled naïve OT I. CD8⁺ T cells were primed with irradiated DC_{OVA} and aT_{EXO} for two days in vitro and stained for CD8, CD25, CD44, CD62L and IL-7R, respectively. Dot plots of CFSE-positive CD8⁺ T cells stained with PE-anti-CD8 Ab are shown, indicating that the CFSE-labeled CD8⁺ T cells underwent some cycles of cell division, and were sorted by flow cytometry for assessment of CD25, CD44, CD62L and IL-7R expression using PE-labeled Abs (solid lines) or PE-isotype matched irrelevant Abs (dotted lines) by flow cytometry. (d) The in vitro DC_{OVA}- and aT_{EXO}-activated OT I CD8⁺ CD45.1⁺ T cells were purified using biotin-anti-CD45.1 Ab and anti-biotin-microbeads (Miltenyi Biotech) and referred to as DC_{OVA}/OT I_{6.1} and aT_{EXO}/OT I_{6.1}, respectively. They were then incubated with irradiated (4,000 rad) EG7 and EL4 for 24 hr. The supernatants in wells containing DC_{OVA}/OT I_{6.1} plus EG7 or EL4 cells (DC_{OVA}/OT I_{6.1}:EG7 or DC_{OVA}/OT I_{6.1}:EL4) and aT_{EXO}/OT I_{6.1} plus EG7 or EL4 cells (aT_{EXO}/OT I_{6.1}:EG7 or aT_{EXO}/OT I_{6.1}:EL4) were examined for IFN-γ expression by ELISA. (e) T cell proliferation assay. In vitro DC_{OVA}- and aT_{EXO}-activated CD8⁺ CD45.1+T cells (0.4×10⁵ cells/well) derived from OT I/B6.1 mice OTI CD8⁺ T cells, primed on day 0 with irradiated DC_{OVA} (▪) or aT_{EXO} (▴) were maintained in cultures for one week with the indicated cytokines [IL-2 (50 U/ml), IL-7 (10 ng/ml) and IL-15 (5 ng/ml)] added on days 3 and 5. Live CD8⁺ T cells with trypin blue exclusion for each culture done in triplicate were counted at the indicated time points. (f) In vitro cytotoxicity assay. The above DC_{OVA}/OT I_{6.1} (▪) and aT_{EXO}/OT I_{6.1} (A) cells were used as effector cells, whereas ⁵¹Cr-labeled EG7 or EL4 cells used as target cells in a chromium release assay. One representative experiment of three is displayed.

FIG. 12 shows stimulation of CD8⁺ T cell proliferation and differentiation in vivo. Wild-type C57BL/6 or Ia^{b-/-} gene KO mice were i.v. immunized with irradiated (a) DC_{OVA}, nT_{EXO}, aT_{EXO} and (b) aT_{EXO} with various gene KO, respectively. Six days after immunization, the tail blood samples of immunized mice were incubated with PE-H-2K^{b}/OVAI tetramer and FITC-anti-CD8 Ab, then analyzed by flow cytometry. The value in each panel represents the percentage of tetramer-positive CD8⁺ T cells versus the total CD8⁺ T cell population. The value in parenthesis represents the standard deviation. (c) In in vivo cytotoxicity assay, the above immunized mice were i.v. co-injected at 1:1 ratio of splenocytes labeled with high (3.0 μM, CFSE^{high}) and low (0.6 μM, CFSE^{low}) concentrations of CFSE and pulsed with OVAI and Mut1 peptide, respectively, six days after immunization with aT_{EXO} and aT_{EXO} with various gene KO, respectively. Sixteen hours after target cell delivery, the residual CFSE^{high} and CFSE^{low} target cells remaining in the recipients' spleens were sorted and analyzed by flow cytometry. The value in each panel represents the percentage of CFSE^{high} cells versus CFSE^{low} cells remaining in the spleens. One representative experiment of three in the above different experiments is shown.

FIG. 13 shows breaking immune tolerance with EXO-targeted CD4⁺ T cells in RIP-mOVA transgenic mice. (a) Proliferation assay. Wild-type C57BL/6 (B6) mice were s.c. immunized with OVAII peptide in CFA (▪) or CFA (∘) alone. (b) RIP-mOVA transgenic mice which had been treated with i.p. injection of anti-CD25 Ab (▪) or the irrelevant control Ab (∘) (0.25 mg/mouse) four days ago were s.c. immunized with OVAII peptide in CFA. Draining lymph nodes were taken from RIP-mOVA mice 10 days after the immunizations. Single cell suspensions were prepared. Serial dilution of OVAII peptide were mixed with 4×10⁵ cells per well in microtiter plates in total volumes of 200 μl/well of RPMI 1640 containing 1% syngenic mouse serum. Four days later, the proliferation response of CD4⁺ T cells was determined by ³H-thymidine uptake assay. (c) Tetramer staining assay. Wild-type C57BL/6(B6) and RIP-mOVA transgenic mice were i.v. immunized with irradiated (4,000 rad) DC_{OVA}, nT_{EXO} and aT_{EXO} cells (3×10⁶ cells/mouse), respectively. Six days after immunization, the tail blood samples of immunized mice were incubated with PE-H-2 K^{b}/OVAI tetramer and FITC-anti-CD8 Ab, then analyzed by flow cytometry. The value in each panel represents the percentage of tetramer-positive CD8⁺ T cells versus the total CD8⁺ T cell population. The value in parenthesis represents the standard deviation. (d) RIP-mOVA transgenic mice were i.v. immunized with irradiated (4,000 rad) DC_{OVA}, nT_{EXO} and aT_{EXO} cells (3×10⁶ cells/mouse), respectively. Mice were monitored for diabetes from day 6 for at least 20 days by urine glucose testing. Animals were considered diabetic after 2 consecutive days with readings ≧56 mmol/L. One representative experiment of three in the above different experiments is shown.

FIG. 14 shows the development of antigen-specific CD8⁺ memory T cells. (a). C57BL/6 mice were immunized with irradiated DC_{OVA} and aT_{EXO}, respectively. Three months later, the tail blood were taken from these immunized mice and stained with PE-H-2 K^{b}/OVA tetramer, FITC-anti-CD8 and ECD-anti-CD44 Abs, and analyzed by flow cytometry. The value in each panel represents the percentage of tetramer-positive CD8⁺ T cells versus the total CD8⁺ T cell population. The value in parenthesis represents the standard deviation. The PE-tetramer and FITC-CD8 positive cells in the squares were sorted and analyzed, showing they were also PE-tetramer and ECD-CD44 positive cells in the circles. (b). The above immunized mice were boosted with DC_{OVA}. Four days after the boost, the recall responses were examined using staining with PE-H-2K^{b}/OVA tetramer and FITC-anti-CD8 Ab and analyzed by flow cytometry. The value in each panel represents the percentage of tetramer-positive CD8⁺ T cells versus the total CD8⁺ T cell population. The value in parenthesis represents the standard deviation. The results presented are representative of 5 separate mice per group. One representative experiment of three is shown.

FIG. 15 is a phenotypic analysis of DC and DC-derived exosomes. BM-derived mDCs, imDCs and mDC-derived exosomes (solid lines) were stained with a panel of Abs, and then analyzed by flow cytometry. These cells and exosomes were also stained with isotype-matched irrelevant Abs, respectively, and employed as control populations (thin dotted lines). One representative experiment of two is displayed.

FIG. 16 shows exosome uptake by DC. (A) Both mDCs and imDCs with (thick solid lines) and without (thin dotted lines) uptake of EXO_{CFSE} and EXO_{6.1} were analyzed for CFSE and CD45.1 expression by flow cytometry. (B) Both mDCs and imDCs with (thick solid lines) and without (thick dotted lines) uptake of EXO_{OVA} were analyzed for expression of a panel of surface molecules by flow cytometry. Irrelevant isotype-matched Abs were used as controls (thin dotted lines). (C) Both mDCs and imDCs derived from gene KO mice with (thick solid lines) and without (thin dotted lines) uptake of EXO_{OVA} were analyzed for expression of a panel of surface molecules including H-2K^{b}, PMHC I, Ia^{b}, CD40, CD54 and CD80, respectively, by flow cytometry. (D) mDCs derived from H-2K^{b} gene KO mice with and without uptake of EXO_{OVA} were analyzed by fluorescent microscopy. (E) To investigate the molecular mechanisms involved in EXO uptaken by DC, mDC(K^{b-/-}) were incubated with a panel of anti-H-2 K^{b}, Ia^{b}, LFA-1, DC-SIGN and DEC205 Abs, the fusion protein CTLA-4/IgG, CCD, D-mannose, D-glucose, D-fucose, D-glucosamine and EDTA, respectively, on ice for 30 min before and during co-culturing with EXO_{OVA}. DCs were then analyzed for expression of H-2K^{b} molecule by flow cytometry. *, p<0.05 versus cohorts without adding any neutralizing reagent (Student's t test). One representative experiment of two is displayed.

FIG. 17 shows the stimulation of T cell proliferation in vitro. (A) In vitro CD8⁺ cell proliferation assay. EXO_{OVA} (10 μg/ml), DC_{OVA}, mDC_{EXO} and imDC_{EXO} (0.3×10⁵ cells/well) and their 2-fold dilutions were co-cultured with a constant number of OT I CD8⁺ T cells (1×10⁵ cells/well). After two days, the proliferation response of CD8⁺ T cells was determined by ³H-thymidine uptake assay. (B) The impact of mDC_{EXO} stimulation of OT I CD8⁺ T cell proliferation by adding each of the neutralizing reagents, a mixture of neutralizing reagents together (mixed reagents), and a mixture of control Abs and fusion proteins (control reagents) was assessed. *, p<0.05 versus cohorts without adding any neutralizing reagent (Student's t test). One representative experiment of three is displayed.

FIG. 18 shows the stimulation of T cell proliferation in vivo. (A) Mice were immunized i.v. with EXO_{OVA}, irradiated DC_{OVA}, mDC_{EXO} and imDC_{EXO}, respectively. After 3, 5, 7 and 9 days of the immunization, the splenocytes were prepared from these immunized mice and assayed for IFN-γ-secreting CD8⁺ T cells in response to OVA I stimulation in Elispot assay. (B) After 3, 5, 7 and 9 days of the immunization, the tail blood samples were taken from these immunized mice and stained with PE-H-2K^{b}/OVA tetramer and FITC-anti-CD8 Ab. The expression of PE-H-2K^{b}/OVA tetramer-specific TCR and CD8 molecules was examined by flow cytometry. (C) A typical flow cytometric analysis of the tail blood samples taken from the wild-type C57BL/6 (B6) and CD4 KO mice 7 days after the immunization was shown. The results presented are representative of 4 separate mice per group. One representative experiment of three is shown.

FIG. 19 shows the development of antigen-specific CTL activities in vitro and in vivo. (A) In vitro cytotoxicity assay, naïve OTI CD8⁺ T cells (2×10⁵ cells/mL) were stimulated for 3 days with EXO_{OVA} (10 μg/mL) or irradiated (4,000 rads) DC_{OVA}, mDC_{EXO} and imDC_{EXO} (0.6×10⁵ cells/ml). These activated CD8⁺ T cells were used as effector (E) cells, whereas ⁵¹Cr-labeled EG7 or control EL-4 tumor cells were used as target (T) cells. Specific killing was calculated as: 100×[(experimental cpm-spontaneous cpm)/(maximal cpm-spontaneous cpm)], as previously described. The data are presented as the percent specific target cell lysis in ⁵¹Cr release assay. Each point represents the mean of triplicate cultures. (B) In in vivo cytotoxicity assay, C57BL/6 splenocytes were harvested from naïve mouse spleens and incubated with either high (3.0 μM, CFSE^{high}) or low (0.6 μM, CFSE^{low}) concentrations of CFSE, to generate differentially labeled target cells. The CFSE^{high} cells were pulsed with OVA I peptide, whereas the CFSE^{low} cells were pulsed with Mut 1 peptide and served as internal controls. These peptide-pulsed target cells were i.v. injected at 1:1 ratio into the above immunized mice 3, 5, 7 and 9 days after immunization of EXO_{OVA}, DC_{OVA}, mDC_{EXO} and imDC_{EXO}, respectively. Sixteen hrs later, the spleens of immunized mice were removed and residual CFSE^{high} and CFSE^{low} target cells remaining in the recipients' spleens were analyzed by flow cytometry. (C) A typical flow cytometric analysis of the splenocytes from the mice 7 days after the immunization was shown. The value in each panel represents the percentage of CFSE^{high} cells versus CFSE^{low} cells remaining in the spleens. One representative experiment of three is shown.

FIG. 20 shows the development of antigen-specific CD8+ memory T cells. (A) C57BL/6 mice were immunized with EXO_{OVA}, DC_{OVA}, mDC_{EXO} and imDC_{EXO}, respectively. Three months later, the tail blood samples were taken from these immunized mice and stained with PE-H-2K^{b}/OVA tetramer and FITC-anti-CD8 Ab or ECD-anti-CD44 Ab, and analyzed by flow cytometry. The PE-tetramer-positive T cells are also ECD-CD44 positive in each respective group assessed by flow cytometric sorting analysis. (B) The above immunized mice were boosted with DC_{OVA}. Four days after the boost, the recall responses were examined using staining with PE-H-2K^{b}/OVA tetramer and FITC-anti-CD8 Ab and analyzed by flow cytometry. The results presented are representative of 4 separate mice per group. One representative experiment of three is shown.

### DETAILED DESCRIPTION OF THE INVENTION

The inventor has demonstrated that T helper cells can acquire antigen-presenting machinery from antigen presenting cells. In particular, the T helper cells can acquire MHC class I/peptide complexes, MHC class I/peptide complexes and co-stimulatory molecules from antigen presenting cells. The inventor has demonstrated that these molecules are functional on the T helper cells. Thus the T helper cells can act as T helper-antigen presenting cells and directly stimulate the immune response, particularly CTL activity.

Accordingly, the invention provides a method of making a T helper-antigen presenting cell comprising contacting an exosome derived from a dendritic cell with a CD4⁺ T cell under conditions that allow absorption of the exosome on the CD4⁺ T cell.

The term “T helper-antigen presenting cells” refers to CD4⁺ T helper cells that can stimulate cytotoxic T lymphocytes by acting as antigen presenting cells. In one embodiment, the T helper-antigen presenting cells express MHC/antigen complexes and co-stimulatory molecules, such as CD54 and CD80, and can act as antigen presenting cells to stimulate cytotoxic T lymphocytes responses. The T helper cells can acquire the MHC/antigen complexes and co-stimulatory molecules directly or indirectly from antigen presenting cells, such as dendritic cells, B cells and macrophages. T helper-antigen presenting cells are also referred to as Th-APCs herein.

T cells express MHC class I and CD54, and some activated T cells have been shown to express MHC class II and CD80. However, Th-APCs differ from these T cells because they express increased levels of MHC class I, MHC class II, CD54 and CD80 molecules as compared to other T cells, and the increased expression is not due to endogenous T cell up-regulation of these molecules. Further, Th-APCs are able to stimulate or enhance the immune system in vitro and in vivo.

The term “exosome” as used herein refers to membrane vesicles that are normally about 50-90 nm in diameter. In the methods of the invention, the exosomes are derived from antigen presenting cells, such as dendritic cells. Exosomes derived from antigen presenting cells, such as dendritic cells, contain antigen presenting machinery, adhesion and costimulatory molecules, including MHC class I/antigen complexes, MHC class II/antigen complexes, CD1, hsp70-90, CD9, CD63, CD81, CD11b, CD11c, CD40, CD54, CD80, CD86, chemokine receptor CCR7, mannose-rich C-type lectin receptor DEC205 and Toll-like receptors TLR4 and TLR9.

The term “exosome derived from a dendritic cell” as used herein refers to preparing and purifying exosomes from a dendritic cell. In one example, a culture of dendritic cells is centrifuged to remove the cells and cellular debris, and then centrifuged to pellet the exosomes. In one embodiment of the invention, the exosome derived from the dendritic cell is from a bone marrow derived dendritic cell.

The term “under conditions that allow absorption of the exosome on the CD4+ T cell” as used herein refers to allowing the exosome and the CD4+ T cells to contact so that the exosome is absorbed on the CD4+ T cell or so that the antigen presenting machinery and/or costimulatory molecules are transferred from the exosome onto the CD4+ T cell. In one embodiment, the exosomes and CD4+ T cells are incubated together at 37° C. for 4 hours. A person skilled in the art will appreciate that the conditions for optimal absorption can depend on a number of factors including, temperature, the concentration of cells, concentration of exosomes, and the composition of the incubation medium.

In one embodiment of the invention the CD4+ T cell is activated prior to contact with the exosome. In another embodiment of the invention, the CD4+ T cell is naïve.

In further embodiment of the invention, the dendritic cell is exposed to an antigen prior to deriving the exosome from the dendritic cell. For example, the dendritic cells can be pulsed with an antigen, such as antigen from an infectious agent or a tumor antigen.

Another aspect of the invention is a method of making a T helper-antigen presenting cell comprising contacting a CD4⁺ T cell with an activated dendritic cell under conditions that allow for transfer of molecules from the dendritic cell to the CD4⁺ T cell. In one embodiment, CD4+ T cells are isolated and then incubated in the presence of dendritic cells for 3 days. In a preferred embodiment, the dendritic cells are bone marrow derived and are activated. In another embodiment, the CD4+ T cells and the dendritic cells are incubated in the presence of IL-2, IL-12 and/or anti-IL-4 antibodies. A person skilled in the art will appreciate that different conditions can be used to allow optimal transfer of molecules from the dendritic cells to the CD4+ T cells. For example, the concentration cells, length of incubation, type of incubation medium, temperature, etc. can be varied.

The transfer of molecules from the dendritic cell to the CD4+ T cell includes the transfer of antigen presentation machinery and/or costimulatory molecules, including, without limitation, MHC class I and peptide complexes, MHC class II and peptide complexes, CD54 and CD80.

Activated dendritic cells can be isolated using methods known to persons skilled in the art (29). In one embodiment, the activated dendritic cells are exposed to an antigen prior to contact with the CD4+ T cell. For example, the dendritic cell can be pulsed with an antigen, such as antigen from an infectious agent or a tumor antigen.

The invention also includes an isolated T helper-antigen presenting cell made according to the methods of the invention. The term “isolated” as used herein refers to a T helper-antigen presenting cell that is substantially free of other cell types, cellular debris or culture medium.

The term “a cell” as used herein includes a single cell as well as a plurality or population of cells.

A person skilled in the art will appreciate that T helper-antigen presenting cells can also be generated by recombinant technology. In one embodiment, T helper cells are genetically engineered to express MHC complexes with an antigen of interest and co-stimulatory molecules, such as CD54 and CD80.

A person skilled in the art will also appreciate that the antigen presenting cells, such as dendritic cells, which are the source of the exosomes can be modified by recombinant technology to express increased levels of antigen presenting machinery, adhesion and/or costimulatory molecules, including MHC class I/antigen complexes, MHC class II/antigen complexes, CD1, hsp70-90, CD9, CD63, CD81, CD11b, CD11c, CD40, CD54, CD80, CD86, chemokine receptor CCR7, mannose-rich C-type lectin receptor DEC205 and Toll-like receptors TLR4 and TLR9. These antigen presenting cells can also be recombinantly engineered to express antigens, such as tumor antigens or antigens from infectious agents, such as viruses and bacteria. The exosomes derived from these recombinantly engineered antigen presenting cells will express these additional molecules and can transfer them to the T helper cells or dendritic cells upon absorption.

Necessary techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991).

The invention also provides methods of enhancing the immune response to treat or prevent a disease comprising administering an effective amount of T helper-antigen presenting cell to an animal in need thereof. The present invention also provides a use of an effective amount of T helper-antigen presenting cells to treat or prevent a disease.

The term “disease” term disease as used herein includes, and is not limited to, cancer, immune diseases, such as an autoimmune disease, or infections.

As used herein, the phrase “to treat or prevent a disease” refers to inhibition or reducing the occurrence of a disease. For example, if the disease is cancer “preventing cancer” refers to prevention of cancer cell replication, inhibition of cancer spread (metastasis), inhibition of tumor growth, reduction of cancer cell number or tumor growth, decrease in the malignant grade of a cancer (e.g., increased differentiation), or improved cancer-related symptoms; and “treating cancer” refers to preventative treatment which decreases the risk of a patient from developing a cancer, or inhibits progression of a pre-cancerous state (e.g. a colon polyp) to actual malignancy. If the disease is an infection, then “preventing infection” refers to prevention or inhibition of the infection, a decrease in the severity of the infection or improved symptoms; and “treating infection” refers to preventative treatment which decreases the risk of a patient from developing an infection, or inhibits the progression or severity of an infection.

As used herein, the phrase “effective amount” means an amount effective, at dosages and for periods of time necessary to achieve the desired result, e.g. to treat or prevent a disease. Effective amounts of T helper-antigen presenting cells may vary according to factors such as the disease state, age, sex, weight of the animal. Dosage regime may be adjusted to provide the optimum therapeutic response. For example, several divided doses may be administered daily or the dose may be proportionally reduced as indicated by the exigencies of the therapeutic situation.

As used herein, the term “animal” includes all members of the animal kingdom, including humans.

The term “enhancing the immune response” as used herein refers to enhancing the immune system of an animal. In a preferred embodiment, the CTL response is enhanced. The immune response of an animal can be readily tested using techniques known in the art. In one embodiment, in vivo or in vitro CD8⁺ T cell proliferation assays can be used. In another embodiment, in vivo or in vitro CD8⁺ cytotoxic assays can be used.

In one embodiment, T helper-antigen presenting cells are used alone to enhance the immune response to treat or prevent a disease. In another embodiment, T helper-antigen presenting cells are used in combination with other immune cells to enhance the immune response to treat or prevent a disease. Other immune cells include, and are not limited to, dendritic cells, macrophages, B cells and cytotoxic T lymphocytes.

In a further embodiment, the method of the invention includes the use of an immune adjuvant. Immune adjuvants are known to persons skilled in the art and include, without being limited to, the lipid-A portion of a gram negative bacteria endotoxin, trehalose dimycolate or mycobacteria, phospholipid bromide (DDA), certain linear polyoxypropylene-polyoxyethylene (POP-POE) block polymers, mineral salts such as aluminum hydroxide, liposomes, cytokines and inert vehicles such as gold particles.

The T helper-antigen presenting cells may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation.

Accordingly, the present invention provides a pharmaceutical composition for preventing or treating a disease comprising an effective amount of T helper-antigen presenting cells and a pharmaceutically acceptable carrier, diluent or excipient.

The active substance may be administered in a convenient manner such as by injection (subcutaneous, intravenous, intramuscular, etc.), oral administration, inhalation, transdermal administration (such as topical cream or ointment, etc.), or suppository applications. Depending on the route of administration, the active substance may be coated in a material to protect the T helper-antigen presenting cells from the action of enzymes, acids and other natural conditions which may inactivate the T helper-antigen presenting cells.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, Remington's Pharmaceutical Sciences (2003-20th edition) and in The United States Pharmacopeia: The National Formulary (USP 24 NF19) published in 1999. On this basis, the compositions include, albeit not exclusively, solutions of the substances in association with one or more pharmaceutically acceptable vehicles or diluents, and contained in buffered solutions with a suitable pH and iso-osmotic with the physiological fluids.

The inventor has also shown that the exosomes derived from dendritic cells can be absorbed onto dendritic cells, particularly mature dendritic cells. These exosome-absorbed dendritic cells express high levels of peptide/MHC class I complexes and costimulatory CD40, CD54, and CD80 molecules. These exosome-absorbed dendritic cells are potent stimulators of the immune system in vitro and in vivo, particularly the CTL response.

Accordingly, another aspect of the invention is a method of making exosome-absorbed dendritic cells comprising contacting an exosome derived from a first dendritic cell with a second dendritic cell under conditions that allow absorption of the exosome on the second dendritic cell.

The phrase “conditions that allow absorption of the exosome” as used herein refers to allowing the exosome and the second dendritic cell to contact so that the exosome is absorbed on the second dendritic cell or so that the antigen presenting machinery and/or costimulatory molecules are transferred from the exosome to the second dendritic cell. In one embodiment, the dendritic cell and exosome are co-cultured for 6 hours at 37° C. A person skilled in the art will appreciate that the conditions for optimal absorption can depend on a number of factors including, temperature, the concentration of cells, concentration of exosomes, and the composition of the incubation medium.

In one embodiment of the invention the first dendritic cell is bone marrow derived. In another embodiment of the invention the second dendritic cell is a mature dendritic cell. In an additional embodiment of the invention, the first dendritic cell is exposed to an antigen prior to deriving the exosome from the dendritic cell. For example, the dendritic cells can be pulsed with an antigen, such as antigen from an infectious agent or a tumor antigen.

The invention also includes the isolated exosome-absorbed dendritic cell made according to the methods of the invention.

The invention also provides methods of enhancing the immune response to treat or prevent a disease comprising administering an effective amount of an exosome-absorbed dendritic cell to an animal in need thereof. As explained above, the term “disease” includes, without limitation, cancer, immune diseases, such as autoimmune diseases, or infections.

The exosome-absorbed dendritic cells can be used alone to enhance the immune response to treat or prevent a disease. In another embodiment, T helper-antigen presenting cells are used in combination with other immune cells to enhance the immune response to treat or prevent a disease. Other immune cells include, and are not limited to, dendritic cells, macrophages, B cells and cytotoxic T lymphocytes. In a further embodiment, the invention includes the use of an immune adjuvant.

The exosome-absorbed dendritic cells can be formulated into pharmaceutical compositions for administration to subjects in a biologically compatible form suitable for administration in vivo.

Accordingly, the present invention provides a pharmaceutical composition for preventing or treating a disease comprising an effective amount of an exosome-absorbed dendritic cell and a pharmaceutically acceptable carrier, diluent or excipient. The pharmaceutical composition can be administered and prepared as described above.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present invention:

### EXAMPLES

### Example 1

### CD4+ T Helper-Antigen Presenting Cells

Materials and Methods

Tumor Cells, Reagents and Animals

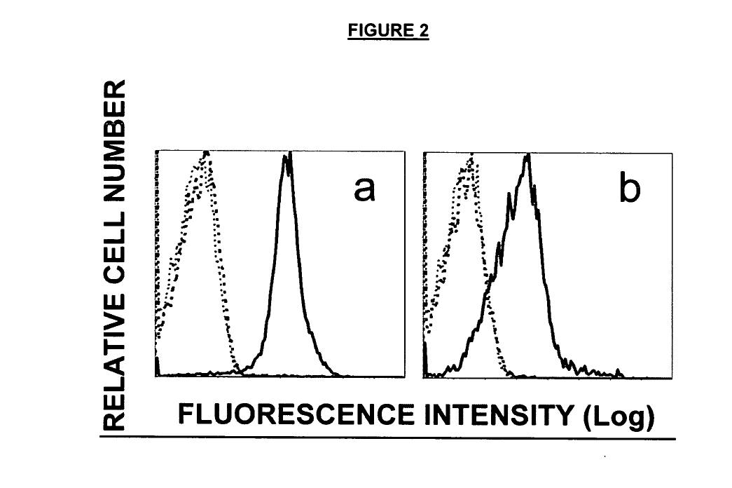

The highly lung metastatic B16 mouse melanoma BL6-10 and OVA-transfected BL6-10 (BL6-10_{OVA}) cell lines were generated by the inventor (30). Both cell lines form numerous lung metastasis after i.v. tumor cell (0.5×10⁶ cells/mouse) injection. The mouse B cell hybridoma cell line LB27 expressing both H-2K^{b} and Ia^{b}, the mouse thymoma cell line EL4 of C57BL/6 mice and the OVA-transfected EL4 (EG7) cell line which is sensitive to CTL killing were obtained from American Type Culture Collection (ATCC, Rockville, Md.). Both BL6-10 and BL6-10_{OVA} express similar levels of H-2K^{b}, but not Ia^{b}. Both BL6-10_{OVA} and EG7 cells expressed OVA by flow cytometric analysis, whereas BL6-10 and EL4 cells did not (FIG. 2). T cell hybridoma cell line RF3370 expresses TCR specific for H-2K^{b}/OVA peptide complexes (31). The biotin-labeled monoclonal Abs specific for H-2K^{b} (AF6-88.5), Ia^{b} (AF6-120.1), CD3 (145-2C11), CD4 (GK1.5), CD8 (53-6.7), CD11b (MAC-1), CD11c (HL3), CD25 (7D4), CD54 (3E2), CD69 (H1.2F3), CD80 (16-10A1) and Vα2Vβ5⁺ TCR (MR9-4) were obtained from BD Pharmingen, Mississauga, ON, Canada. The OVAI (SIINFEKL) (SEQ ID NO:1) and OVAII (ISQAVHAAHAEINEAGR) (SEQ ID NO:2) peptides (32,33) are OVA tumor peptides for H-2K^{b} and Ia^{b}, respectively, whereas Mut1 (FEQNTAQP) (SEQ ID NO:3) peptide is an irrelevant 3LL lung carcinoma for H-2K^{b} (34). These peptides were synthesized by Multiple Peptide Systems (San Diego, Calif.). The OVA-specific TCR transgenic OT I and OT II mice, and H-2 K^{b}, Ia^{b}, CD4, CD8, CD54 and CD80 KO mice on a C57BL/6 background were obtained from the Jackson Laboratory (Bar Harbor, Mass.). Homozygous OT II/H-2K^{b-/-}, OT II/Ia^{b-/-}, OT II/CD54^{-/-} and OT II/CD80^{-/-} mice were generated by backcrossing the designated gene KO mice (H-2K^{b}) onto the OT II background for three generations; homozygosity was confirmed by PCR according to Jackson laboratory's protocols. All mice were maintained in the animal facility at the Saskatoon Cancer Center and treated according to animal care committee guidelines of University of Saskatchewan.

Preparation of Dendritic Cells

Activated, mature bone marrow-derived DCs, expressing high levels of MHC class II, CD40, CD54 and CD80, were generated from C57BL/6 mice, as described previously (29). To generate OVA-pulsed DC (DC_{OVA}), DCs were pulsed overnight at 37° C. with 0.1 mg/ml OVA (Sigma, St. Louis, Mo.), then washed extensively (34).

Preparation of OT II CD4⁺ and OT I CD8⁺ T Cells

Naïve OVA-specific CD4⁺ T and CD8⁺ T cells were isolated from OT II or OT I mouse spleens, respectively, and enriched by passage through nylon wool columns. CD4⁺ and CD8⁺ cells were then purified by negative selection using anti-mouse CD8 (Ly2) or CD4 (L3T4) paramagnetic beads (DYNAL Inc, Lake Success, N.Y.) to yield populations that were >98% CD4⁺/Vα2Vβ5⁺ or CD8⁺/Vα2Vβ5⁺, respectively. To generate DC_{OVA}-activated CD4⁺ T cells, CD4⁺ T cells (2×10⁵ cells/ml) from OT II mice or designated gene-deleted OT II mice were stimulated for three days with irradiated (4,000 rads) BM-derived DC_{OVA} (1×10⁵ cells/ml) in the presence of IL-2 (10 U/ml), IL-12 (5 ng/ml) and anti-IL-4 antibody (10 μg/ml) (R&D Systems, Minneapolis, Minn.) (35). These in vitro DC_{OVA}-activated CD4⁺ T cells, also referred to herein as CD4⁺ Th-Ag presenting cells (Th-APCs), were then isolated by Ficoll-Paque (Sigma) density gradient centrifugation, or further purified using CD4 microbeads (Milttenyi Biotec, Auburn, Calif.) in some experiments. Con A-stimulated OT II CD4⁺ T (Con A-OT II) cells were similarly generated by incubating splenocytes from OT II or OT Il/KO mice with Con A (1 μg/ml) and IL-2 (10 U/ml) for 3 days, after which the CD4⁺ T cells were purified on density gradients. To ascertain that no DCs were in purified Th-APCs or Con A-OT II cells, these active T cells were further purified by using CD4 microbeads (Milttenyi Biotec).

Phenotypic Characterization of DC_{OVA}-Activated CD4⁺ T Cells

For the phenotypic analyses, Th-APCs were stained with Abs specific for H-2K^{b}, Ia^{b}, CD3, CD4, CD8, CD11b, CD11c, CD25, CD54, CD69, CD80 and Vα2Vβ5⁺ TCR (BD Pharmingen), respectively, and analyzed by flow cytometry. For the intracellular cytokines, cells were restimulated with 4000 rad-irradiated BL27 tumor cells pulsed with OVAII peptide for 4 hours (35), and then processed using a ‘Cytofix/CytoPerm Plus with GolgiPlug’ kit (BD Pharmingen), with R-phycoerythrin (PE)-conjugated anti-IL4, -perforin and -IFN-γ Abs (R&D Systems), respectively. Culture supernatants of the re-stimulated Th-APCs were analyzed for IFN-γ, IL-2 and IL-4 expression using ELISA kits (Endogen, Cambridge, Mass.), as reported previously (34).

In Vitro and In Vivo Membrane Molecule Transfer Assays

In in vitro membrane transfer assay, DC_{OVA} or DC were incubated with 5-carboxy-fluorescein diacetate succinimidyl ester (CFSE; 0.5 μM) at 37° C. for 15 minutes and washed 3 times with PBS. CFSE-labeled DC_{OVA} or DC were incubated with Con A-OT II cells at 37° C. for 4 hours, then the cell mixtures, the original DC_{OVA} and Con A-OT II cells were stained with a panel of phycoerythrin-Texas red-X (ECD)-Abs specific for H-2 K^{b}, CD54 and CD80, respectively, and analyzed by confocal fluorescence microscopy. CD4⁺ T cells in the cell mixture were also purified by cell sorting and analyzed by flow cytometry. Con A-OT II cells stained with biotin-labeled isotype-matched Abs and ECD-avidin (BD Pharmingen) were used as controls.

In in vivo membrane transfer assay, naïve T cells were isolated from OT II/Ia^{b-/-} and OT II/CD80^{-/-} mouse spleens, respectively, and enriched by passage through nylon wool columns. The CD4⁺ T cells (5×10⁶ cells/mouse) were further purified by negative selection using the anti-mouse CD8 (Ly2) paramagnetic beads (DYNAL Inc), and then i.v. injected into wild-type C57BL/6 mice. One group of mice remained untreated. One day subsequent to the injection, another group of mice were i.v. immunized with irradiated (4,000 rads) DC_{OVA} (0.2×10⁶ cells/mouse). Three days after the immunization, mice were sacrificed. T cells were isolated from the spleens of these two groups of mice, and enriched by passage through nylon wool columns. The OVA-specific CD4⁺ OT II T cells were further purified from these T cells by positive selection using the biotin-anti-TCR antibody and anti-biotin microbeads (Milttenyi Biotec), and then stained with FITC-anti-Ia^{b} and FITC-anti-CD80 antibodies for flow cytometric analysis, respectively.

Antigen Presentation

RF3370 hybridoma cells (0.5×10⁵ cells/well) were cultured with irradiated (4,000 rad) DC_{OVA} or Th-APCs or Con A-OT II (1×10⁵ cells/well) for 24 hr. To investigate the fate of acquired MHC class I/peptide expression, Th-APCs alone were cultured for 1, 2 and 3 days in culture medium containing IL-2 (10 U/ml), termed Th-APC (1, 2 and 3 Day), and then harvested for stimulation of RF3370 cells, respectively. The supernatants were harvested for measurement of IL-2 secretion using ELISA kit (Endogen).

CD8⁺ T Cell Proliferation Assays

For in vitro CD8⁺ T cell proliferation assay, irradiated (4,000 rads) stimulators, the Th-APCs, Con A-OT II cells (0.4×10⁵ cells/well), DC_{OVA} (0.1×10⁵ cells/well) and their 2-fold dilutions were cultured with a constant number of responders, the naïve OT I or C57BL/6 (B6) CD8⁺ T cells (0.5×10⁵ cells/well). To rule out the potent effect of endogenous H-2K^{b}, Th-APCs generated from H-2K^{b-/-} OT II T cells were termed K^{b-/-} Th-APCs and used as stimulators. In some experiments, each of a panel of neutralizing reagents (anti-IL-2, -H-2K^{b} or -LFA-1 Abs, and CTLA-4/Ig fusion protein) (each 15 μg/ml; R&D Systems) or a mixture of the above reagents were added to the cells, while control cells received a mixture of isotype-matched irrelevant Abs and fusion protein. In other experiments, the irradiated CD4⁺ Th-APCs and naïve OT I CD8⁺ T cells were cultured in transwell plates (Costar, Corning, N.Y.), separated by 0.4 μM pore-sized membranes. After 48 hrs, thymidine incorporation was determined by liquid scintillation counting (34).

For in vivo CD8⁺ T cell proliferation assay, purified naïve OT I CD8⁺ T cells were labeled with CFSE (1.5 μM) and i.v. injected into C57BL/6 mice (2×10⁶ cells each). Twelve hours later, each mouse was i.v. injected with 2×10⁶ Th-APCs and Con A-OT II cells, respectively, or 0.2×10⁶ DC_{OVA}. In another group, mice were injected with PBS. Three days later, the splenic T cells from the recipients were stained with ECD-anti-CD8 Ab (Beckman Coulter, Miami, Fla.), and then analyzed by flow cytometry.

Cytotoxicity Assays

For in vitro cytotoxicity assay, the activated CD8⁺ T cells derived from the above three day co-culture with irradiated (4,000 rads) DC_{OVA}, Th-APCs and Con A-OT II cells were purified on density gradients and termed DC_{OVA}/OT I, Th-APC/OT I and Con A-OT II/OT I, respectively. These cells as well as Th-APCs were used as effector (E) cells, while ⁵¹Cr-labeled EG7, the control EL-4 tumor cells, DC_{OVA}, LB27 and OVAII-pulsed LB27 (LB27_{OVAII}) tumor cells were used as target (T) cells, respectively. Specific killing was calculated as: 100×[(experimental cpm−spontaneous cpm)/(maximal cpm−spontaneous cpm)], as previously described (34).

The inventor adapted a recently reported in vivo cytotoxicity assay (36). Briefly, C57BL/6 mice were i.v. immunized with DC_{OVA} (0.5×10⁶ cells), Th-APCs or Con A-OT II cells (2×10⁶ cells). Seven days later, mice were boosted once. In another group, mice were injected with PBS. Naïve mouse splenocytes were incubated with either high (3.0 μM, CFSE^{high}) or low (0.6 μM, CFSE^{low}) concentrations of CFSE, to generate differentially labeled target cells. The CFSE^{high} cells were pulsed with OVAI, whereas the CFSE^{low} cells were pulsed with the irrelevant 3LL lung carcinoma H-2K^{b} peptide Mut1 and served as internal controls. These peptide-pulsed target cells were washed extensively to remove free peptide, and then i.v. co-injected at 1:1 ratio into the above immunized mice three days after the boost. Sixteen hours after target cell delivery, the spleens were removed and residual CFSE^{high} and CFSE^{low} target cells remaining in the recipients' spleens were sorted and analyzed by flow cytometry.

Animal Studies

Wild-type C57BL/6 mice (n=8) were injected i.v. with 0.2×10⁶ DC_{OVA}, 2×10⁶ Th-APCs and Con A-OT II cells, respectively, and then 7 days later they were boosted once. To study the immune mechanism, CD4 and CD8 KO mice (n=8) were injected i.v. with 2×10⁶ Th-APCs, and then 7 days later the mice were boosted once. Three days subsequent to the boost, the mice were i.v. given 0.5×10⁶ BL6-10_{OVA} or BL6-10 tumor cells. The mice were sacrificed 4 weeks after tumor cell injection and the lung metastatic tumor colonies were counted in a blind fashion (30). Metastases on freshly isolated lungs appeared as discrete black pigmented foci that were easily distinguishable from normal lung tissues and confirmed by histological examination. Metastatic foci too numerous to count were assigned an arbitrary value of >100.

Results

CD4⁺ Th-APCs Acquire the Synapse-Composed MHC Class II and CD54 Molecules and the Bystander MHC Class I from APCs by APC Stimulation

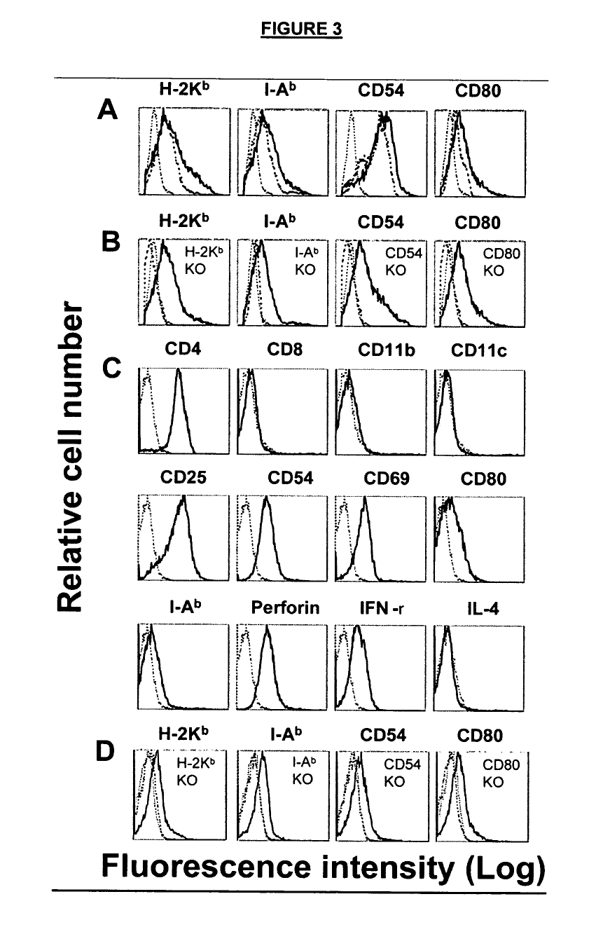

In order to explore DC membrane-derived APC machinery acquisition by CD4⁺ T cells, Con A-stimulated CD4⁺ T cells from OVA-specific TCR transgenic OT II mice were cultured for 4 h either alone or with OVA-pulsed DCs (DC_{OVA}) or DC. The CD4⁺ T cells were then sorted and examined for expression of MHC class I and II, CD54 and CD80 by flow cytometry. The control Con A-stimulated OT II CD4⁺ T cells expressed some MHC class I and II, CD54 and CD80. However, following incubation with DC_{OVA}, these T cells displayed moderately augmented levels of these molecules (FIG. 3A), suggesting that DC molecules could have been transferred to the T cells. The membrane transfer can be mostly blocked by addition of anti-H-2 Kb and LFA-1 antibodies and CTLA-4/Ig fusion protein, indicating that the membrane acquisition of Th-APCs from DC_{OVA} is mediated by TCR and co-stimulatory molecules. In addition, these T cells following interaction with DCs without OVA pulsing also displayed augmented levels of these molecules, but to a lesser extent, indicating that these DC molecule transfer is mediated by both the antigen-specific and non-specific manners.

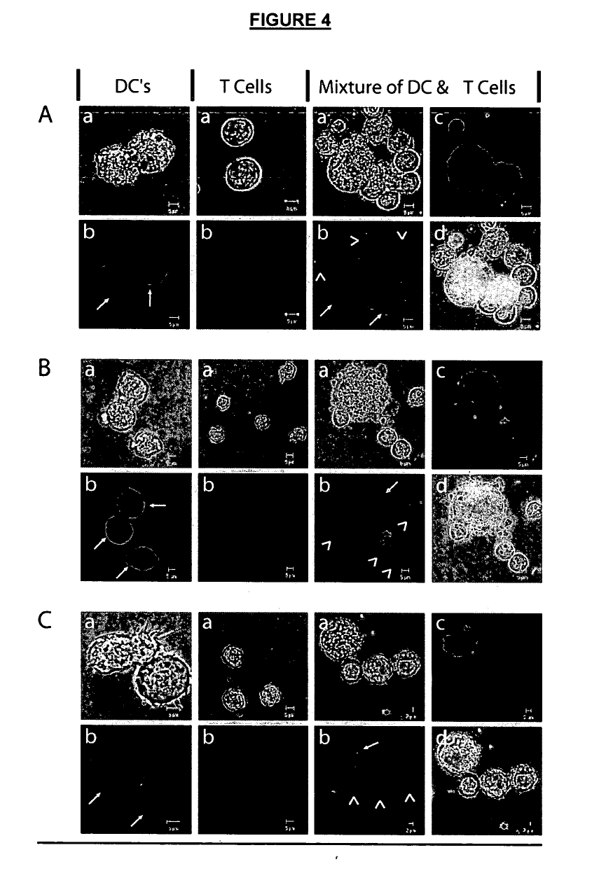

Since all T cells express MHC class I and CD54, and some activated T cells also express MHC class II and CD80 molecules (37,38), it was necessary to confirm that the increased levels of T cell-associated MHC class I and II, CD54 and CD80 were not due to endogenous T cell up-regulation of these molecules. Thus, CFSE-labeled DC_{OVA} with Con A-stimulated CD4⁺ T cells derived from OT II mice were incubated with homozygous H-2K^{b}, Ia^{b}, CD54 and CD80 gene KO, respectively, then sorted the T cells and assessed their expression of these markers. The T cells did not express their respectively deleted gene products when cultured alone, but did discernibly express H-2 K^{b}, Ia^{b}, CD54 and CD80 after 4 hr incubation with DC_{OVA}, as determined by flow cytometry (FIG. 3B) or confocal fluorescence microscopy (FIG. 4). These results indicate that, besides previously reported MHC class I transferred onto CD8⁺ T cells during DC/CD8⁺ T cell interaction and MHC class II and CD80 molecules transferred onto CD4⁺ T cells during DC/CD4⁺ T cell interaction (21,39,40), CD4⁺ T cells can also acquire CD54 forming the immune synapse (18,19) as well as the bystander MHC class I molecules from DCs after DC stimulation of CD4⁺ T cells. In addition to the mechanism of antigen-specific MHC-TCR mediated internalization and recycling (20,21), the uprooting of APC molecules or APC-released vesicles may also contribute to the above membrane transfer, especially the bystander MHC class I (41).

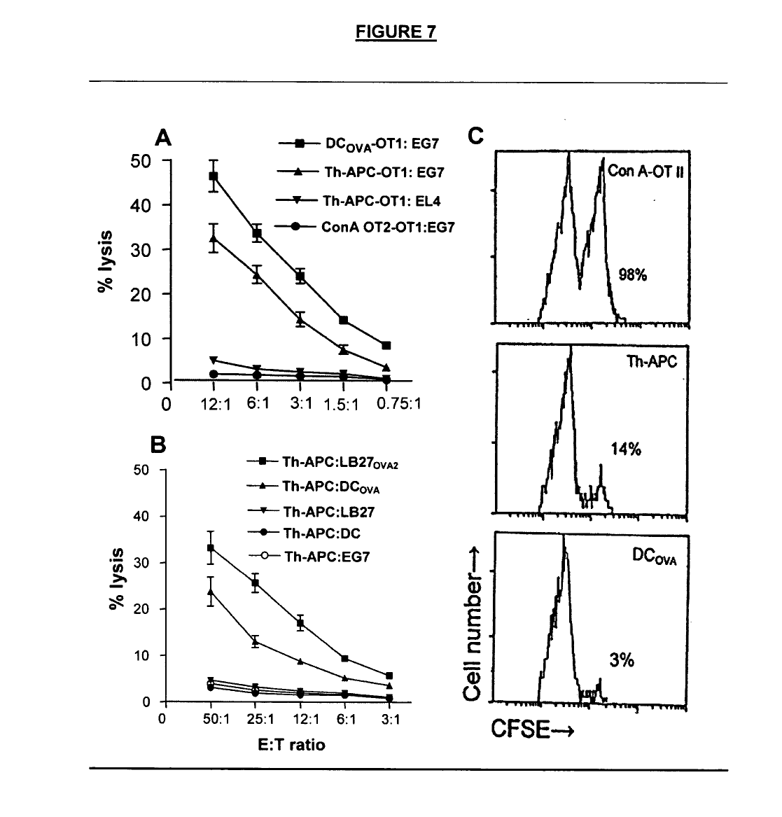

The inventor then examined whether naïve T cells can also acquire DC Ag-presenting machinery in culture. Naïve OT II CD4⁺ T cells were first purified by using nylon column to remove DCs and B cells and anti-CD8 paramagnetic beads (DYNAL Inc) to remove CD8⁺ T cells, and then incubated for three days with irradiated DC_{OVA}. The activated OT II CD4⁺ T cells were then purified by using ficoll-Paque density gradient centrifugation and CD4 microbeads (Milttenyi Biotec), and then analyzed by flow cytometry. These T cells, which proliferated in response to DC_{OVA} stimulation, expressed cell surface CD4, CD25 and CD69, and intracellular perforin and IFN-γ, but not IL-4 (FIG. 3C); they also secreted IFN-γ (˜2 ng/ml/10⁶ cells/24 hr) and IL-2 (˜2.5 ng/ml/10⁶ cells/24 hr), but not IL-4, in culture. This data indicates that these OVA-TCR transgenic CD4⁺ T cells were type 1 T helpers (Th1). In addition, there was no CD11b⁺/11c⁺DC population existing in these purified CD4⁺ T cells (FIG. 3C). This is because that any survival irradiated DC_{OVA} cells and the potential small amount of contamination of spleen DCs or B cells within the original naïve OT II CD4⁺ T cell preparation, which might picked up OVA peptides from irradiated DC_{OVA} in the culture, would be eliminated by the killing activity of these activated Th1 cells expressing perforin (FIG. 7B) (42,43). In addition to the common H-K^{b} expression, these Th cells also expressed Ia^{b}, CD54 and CD80 molecules, and here too they did so whether they were derived from wild-type or homozygous H-2K^{b-/-}, Ia^{b-/-}, CD54^{-/-} or CD80^{-/-} KO mice (FIG. 3D). Thus, the inventor demonstrates that naïve CD4⁺ T cells can also acquire MHC class II and costimulatory molecules (CD54 and CD80) composing the immune synapse as well as the bystander

MHC class I from DCs by In Vitro DC Stimulation.

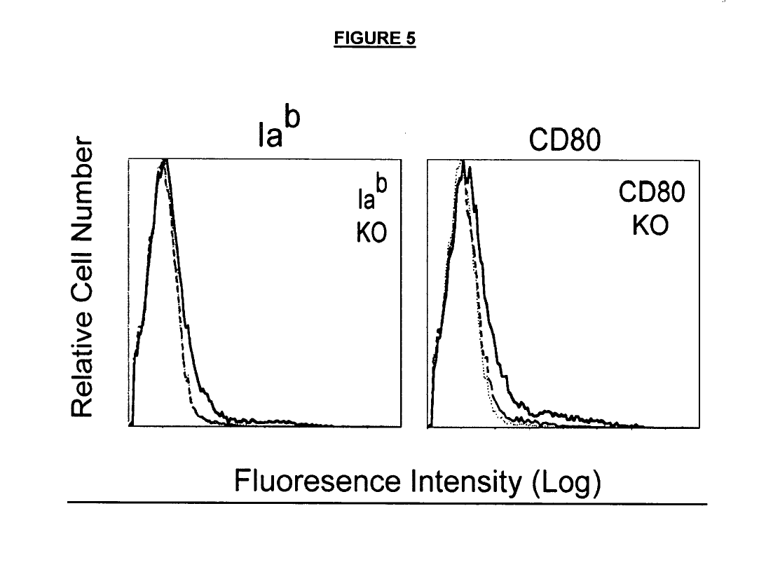

To further confirm the membrane acquisition in vivo, wild-type C57BL/6 mice were first injected with purified CD4⁺ OT II/Ia^{b-/-} and OT II/CD80^{-/-} T cells, and then immunized with DC_{OVA}. Three days after the immunization, mice were sacrificed. CD4⁺ OT II T cells were purified from these immunized mouse spleens, and then stained with FITC-anti-Ia^{b} and FITC-anti-CD80 antibodies for flow cytometric analysis, respectively. As shown in FIG. 5, CD4⁺ OT II/Ia^{b-/-} and OT II/CD80^{-/-} T cells derived from mice immunized with DC_{OVA} became slightly Ia^{b} and CD80 positive, respectively, whereas these T cells derived from mice without immunization remained Ia^{b} and CD80 negative, indicating that CD4⁺ OT II T cells acquire Ia^{b} and CD80 molecules by in vivo DC_{OVA} stimulation.

Th-APCs Stimulate CD8⁺ T Cell Proliferation In Vitro and In Vivo

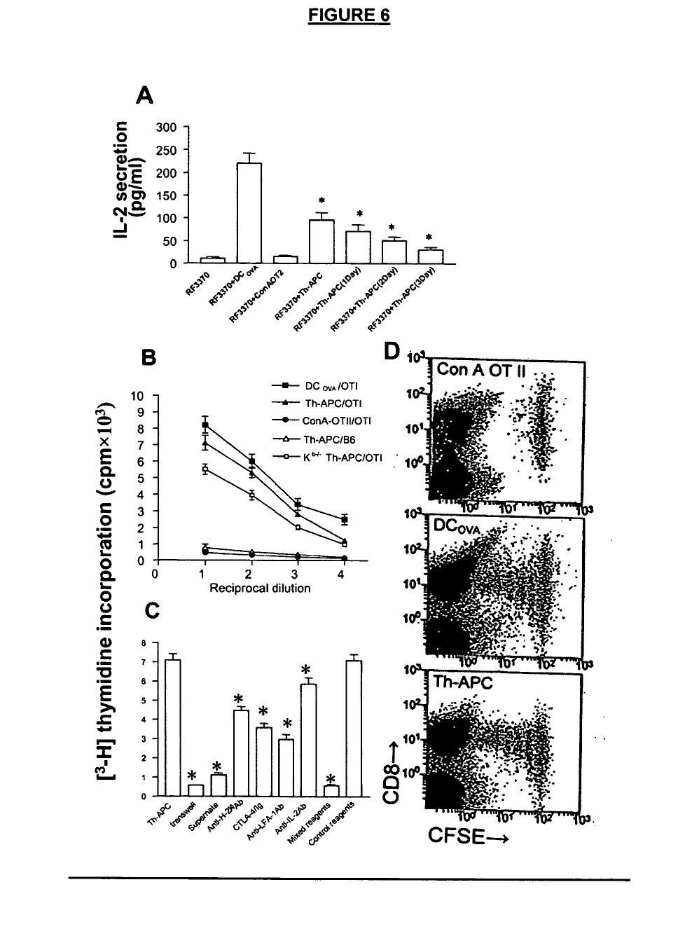

The ability of the CD4⁺ T cells, which acquired H-2K^{b}/OVAI peptide complexes and the DC Costimulatory molecules, to act as direct APCs (termed CD4⁺ TL-APLs) for CD8⁺ T cell stimulation was then examined. To examine the functionality of these putative Th-APC cells, the inventor initially assessed their ability to stimulate IL-2 secretion of T cell hybridoma RF3370. As shown in FIG. 6A, RF3370 cells alone did not secret IL-2. However, Th-APCs significantly stimulated RF3370 to secret IL-2 (95 pg/ml) as did DC_{OVA} (220 pg/ml), indicating that Th-APCs expressed functional H-2K^{b}/OVAI peptide complexes. The stability of the acquired MHC I/OVAI peptide complexes was then assessed. The rate of their decay was assessed by culturing these Th-APCs after MHC class I acquisition for varying time periods. As shown in FIG. 6A, the ability to stimulate IL-2 secretion of RF3370 cells did decay over time. However, readily detectible MHC class I/peptide expression was still observed as much as 3 days after in vitro culture.

To further confirm the results, the inventor then assessed the ability of the Th-APCs to induce proliferation of naïve OT I CD8⁺ T cells in vitro. The positive control DC_{OVA} cells which previously demonstrated to possess a highly activated phenotype (29) strongly induced OT I cell proliferation (FIG. 6B). DC_{OVA}-activated CD4⁺ Th-APCs which were purified by Ficoll-Paque density gradient centrifugation and using CD4 microbeads did indeed stimulate proliferation of OT I CD8⁺ T cells, but to a lesser extent due to (i) less costimulatory molecules and (ii) lacking the third signal, DC-secreted IL-12 (44), compared with DC_{OVA}. However, they did not stimulate responses of the control naïve C57BL/6 (B6) mouse CD8⁺ T cells, nor did Con A-stimulated OT II CD4⁺ T (Con A-OT II) cells [secreting IFN-γ (˜4.0 ng/ml/10⁶ cells/24 hr) and IL-2 (˜3.3 ng/ml/10⁶ cells/24 hr), but lacking self IL-4 and acquired H-2K^{b}/OVA peptide complexes] stimulate OT I CD8⁺ T cell proliferation. In addition, K^{b-/-} Th-APCs derived from the H-2K^{b-/-} OT II KO mice (FIG. 3D) showed similar CD8⁺ T cell stimulatory activity as Th-APCs derived from the wild-type OT II mice (FIG. 6B), indicating that the activation of CD8⁺ OT I T cells is mediated via the acquired H-2K^{b}/OVA peptide complexes, but not the endogenous H-2K^{b} of Th-APCs. In separate experiments, it was demonstrated that CD8⁺ T cell stimulatory activity of the Th-APCs was contact-dependent since transwells blocked CD8⁺ T cell proliferation (FIG. 6C). Furthermore, adding anti-MHC class I or -LFA-1 Abs, or cytotoxic T lymphocyte-associated Ag (CTLA)-4/Ig fusion protein could significantly inhibit the OT I CD8⁺ T cell proliferative response in the co-cultures by 38, 50, and 58%, respectively, while anti-IL-2 antibody had less effect (19% inhibition) (p<0.01). Simultaneous addition of all blocking reagents reduced the proliferative response by 92% (p<0.01). Taken together, this data indicates that this response is critically dependent on H-2K^{b}/OVAI/TCR specificity and greatly affected by nonspecific co-stimulatory CD54/LFA-1 and CD80/CD28 interactions between the CD4⁺ Th-APCs and CD8⁺ T cells. That this proliferative effect was not simply an in vitro artifact was confirmed by demonstrating that these Th1-APCs can also stimulate proliferative responses in vivo. The inventor adoptively transferred CFSE-labeled naïve OT I CD8⁺ T cells into mice that were also given Th-APCs, ConA-OT II cells, DC_{OVA} or PBS. The labeled CD8⁺ T cells did not show any division in mice treated with PBS. However, the labeled CD8⁺ T cells underwent some cycles of cell division in the mice given either Th-APCs or DC_{OVA}, but did not respond in the animals given Con A-OT II cells (FIG. 6D).

Th-APCs Stimulate CD8⁺ T Cell Differentiation into CTL Effectors In Vitro and In Vivo

As a critical test of the functionality of these purified CD4⁺ Th-APCs, their ability to induce the differentiation of naïve OT I CD8⁺ T cells into CTL effectors was tested, as determined using in vitro ⁵¹Cr release assays with EG7 tumor cells expressing an OVA transgene. The Th-APC-activated OT I CD8⁺ T (Th-APC/OT I) cells displayed substantial cytotoxic activity (33% specific killing; E:T ratio, 12) against an OVA-expressing EG7 cell line as did the DC_{OVA}-activated OT I CD8⁺ T (DC_{OVA}/OT I) cells (46% killing; E:T ratio,

12), but not against its parental EL4 tumor cells (FIG. 7A), indicating that the killing activity of these CTLs is OVA-tumor specific. In addition, these CD4⁺ Th-APCs expressing perforin (FIG. 3C) displayed killing activities for DC_{OVA} and LB27_{OVAII} cells with Ia^{b}/OVAII expression (FIG. 7B). However, they themselves did not show any killing activity to LB27 and EG7 (FIG. 7B) or BL6-10_{OVA} cells without Ia^{b}/OVAII expression. As with the proliferation assays, the in vitro CD8⁺ CTL induction capacity of CD4⁺ Th-APCs can also be translated into an induction of effector CTL function in vivo. The inventor adoptively transferred OVAI peptide-pulsed splenocytes that had been strongly labeled with CFSE (CFSE^{high}), as well as the control peptide Mut1-pulsed splenocytes that had been weakly labeled with CFSE (CFSE^{low}), into recipient mice that had been vaccinated with these purified Th-APCs, DC_{OVA}, Con A-OT II cells or PBS. The disappearance of the labeled cells from the mice was assessed by flow cytometric analysis and found that the CFSE^{low} (irrelevant Mut1 peptide-pulsed) cells were unaffected by the vaccination protocol. In addition, no substantial loss (1%) of the CFSE^{high} (OVAI peptide-pulsed) cells from the PBS-immunized mice was found. However, there was substantial loss of the CFSE^{high} (OVAI peptide-pulsed) cells from the Th-APC-immunized (86%) or DC_{OVA}-vaccinated (97%) mice, but not from the Con A-OT II cell-vaccinated (2%) mice (FIG. 7C). These data indicate that CD4+Th-APCs carrying H-2K^{b}/OVAI complexes and DC co-stimulatory molecules can stimulate the development of OVA-specific CTL effector cells in vivo.

Th-APCs Induce OVA-Specific Antitumor Immunity In Vivo

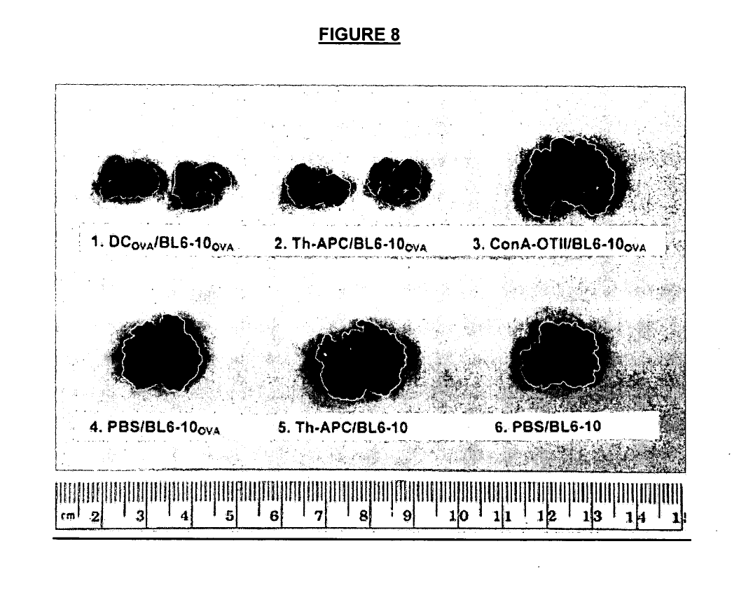

In addition, Th-APCs can also stimulate OVA-specific CTL-mediated antitumor immunity in vivo. These purified Th-APCs were injected i.v. into mice, followed by i.v. challenge with OVA-expressing BL6-10_{OVA} or OVA-negative BL6-10 tumor cells. All mice immunized with Con A-OT II cells (i.e., cells lacking acquired H-2K^{b}/OVAI complexes and co-stimulatory molecules) as well as the control mice (8/8) without any immunization had large numbers (>100) of lung metastatic tumor colonies four weeks after tumor cell challenge (Exp I of Table 1 and FIG. 8). In addition, all mice (8/8) immunized with naïve OT II T cells also died of lung metastasis. However, all mice (8/8) immunized with Th-APCs had no lung tumor metastasis. DC_{OVA} immunization was equally effective in inducing anti-tumor immunity. The specificity of the protection was confirmed with the observation that Th-APCs did not protect against BL6-10 tumors that did not express OVA, with all mice having large numbers (>100) of lung metastatic tumor colonies after tumor cell challenge. To study the immune mechanism, CD4 and CD8 KO mice were used for immunization of Th-APCs. As shown in Exp II of Table 1, all of the CD4 KO mice (8/8) were still protected from BL6-10_{OVA} tumor challenge, indicating that activation of CD8⁺ CTL response by Th-APCs is independent on the host CD4⁺ T cells. However, all CD8 KO mice (8/8) had numerous lung tumor metastases, indicating that the Th-APCs-driven antitumor immunity is mediated by CD8⁺ CTLs. The Th-APC-induced CD8+ CTL response is more likely through direct interaction between Th-APCs and CD8⁺ CTLs rather than cross-presentation of the host DCs picking up OVA peptides released from Th-APCs, because the former is CD4⁺ T cell independent whereas the latter is CD4⁺ T cell dependent.

Discussion

A long-standing paradox in cellular immunology has been the conditional requirement for CD4⁺ Th cells in priming of CD8⁺ CTL responses. CTL responses to non-inflammatory stimuli (e.g., MHC class I alloantigen Qa-1, the male HY Ag) are CD4⁺ T cell-dependent (2,45,46). The inventor demonstrates the critical helper requirement for CTL induction, as have two other recent reports. Wang et al showed that the primary CD8⁺ T cell responses to Ags presented in vivo by peptide-pulsed DCs are also dependent on help from CD4⁺ T cells (47). More importantly, Behrens et al have demonstrated that coinjection of Ag-presenting DC-activated, but not naïve, CD4⁺ OT II T cells induces CTL responses against islet β cell OVA Ag and leads to diabetes in rat insulin promoter (RIP)-OVA^{hi} transgenic mice. They also found that activated CD4⁺ OT II T cells provide CD40-mediated help to CD8⁺ T cell responses without these T cells necessarily seeing Ag on the same APC (48). On the other hand, some have suggested that CD4⁺ T cell help is only essential for memory CTL responses (36). Thus, the generation of effectors from naïve CD8⁺ T cells is reported to be helper independent in mice immunized with irradiated embryonic cells expressing an adenovirus type 5 E1A transgene (49). Having said that it is highly relevant that such adenoviral challenge would also introduce potent inflammatory signals into the sensitizing microenvironment (leading to high level DC maturation) (50), to say nothing of the potential for help from natural killer cells (51). In addition, the E1A adenoviral Ag features multiple CD8⁺ T cells epitopes (52), and therefore also a greater base of Ag-specific CD8⁺ T cell precursors from which to draw (53). A strong and direct activation of DCs (54) would explain the previous demonstrations that induction of some anti-viral CTL responses is CD4⁺ T helper cell-independent.

T cell-to-T cell (T-T) Ag presentation, dependent upon activated CD4⁺ T cells first acquiring MHC class II and CD80 molecules from APCs and then stimulating other CD4⁺ T cells, is increasingly attracting attention (39,40). However, the roles such T-APCs may play in vivo have been as yet ill defined and the results of the relevant in vitro studies disparate, in part because multiple experimental systems have been employed. For example, CD4⁺ T-APCs can induce IL-2 production and proliferative responses among naïve responder T cells (55,56), which is consistent with the results in this study. However, these T-APCs have also been shown to induce apoptosis in activated CD4⁺ T cells or anergization of CD4⁺ T cell lines (40,57-59). In contrast, the inventor found that in vivo transfer of CD4⁺ Th1-APCs expressing high levels of INF-γ and IL-2, which were generated by incubation of OT II CD4⁺ T cells with DC_{OVA} in the presence of IL-12 and anti-IL-4 antibody, were able to stimulate OVA-specific CTL responses. Interesting, the inventor also found that in vivo transfer of CD4⁺ Th2-APCs expressing high levels of IL-4 and IL-10, which were generated by incubation of OT II CD4⁺ T cells with DC_{OVA} in the presence of IL-4 and anti-IFN-γ antibody, were able to induce OVA-specific immune suppression. In other reports, however, in vivo transfer of CD4⁺ Th1-APCs derived from IL-2-dependent transformed T cell lines, has been reported to induce immunosuppressive, but not immunostimulatory effects in the context of autoimmune responses (59,60). In these studies, the T-APCs employed were derived from rather uncharacterized Con A-stimulated allogeneic or Ag-pulsed CD4⁺ T cell lines. Therefore, it is difficult to assess the extent to which they are representative of T-APCs as they would be generated in vivo. In addition, these studies have addressed only the activation of CD4⁺ T cell responses.

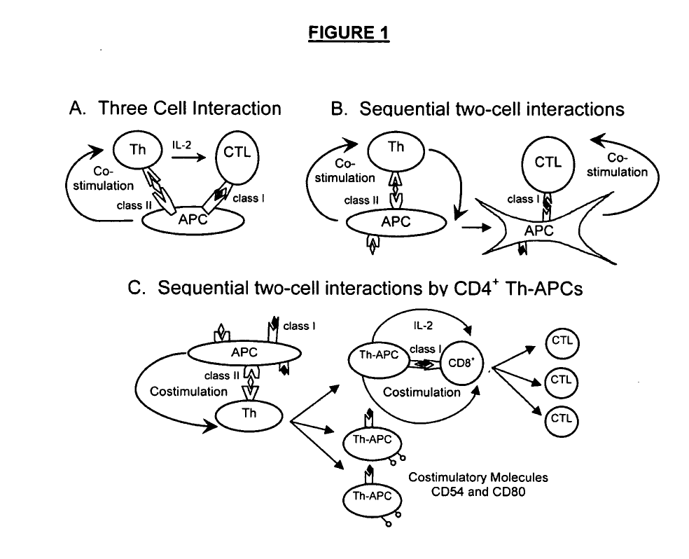

In this study, it was shown that CD4⁺ T cells can acquire synapse-composed MHC class II, CD54 and CD80 molecules from APCs by APC stimulation. In addition, for the first time, the inventor has shown that CD4⁺ T cells can also acquire the bystander MHC class I/OVAI peptide complexes which are critical molecules in stimulation of OVA-specific CTL responses. Furthermore, the inventor has provided a complete line of evidence that compellingly substantiates the practical aspects of CD4⁺ T cells acting as APCs for effective CD8⁺ T cell responses in vitro and in vivo. A model of CD4⁺ T cell help for CTL induction that takes these observations into account would address multiple important aspects of this paradigm in cellular immunology. A central caveat in models of CD4⁺ T cell help for CTL responses is that of scarcity, or how rare Ag peptide-carrying DCs, Ag-specific CD4+, and Ag-specific CD8⁺ T cells manage to encounter each other with enough efficiency to ensure that we expeditiously and appropriately respond to all Ags/pathogens (i.e., to maintain the integrity of the organism). It is counter-intuitive that a function as critical as this not be optimized in some way. The model wherein APCs that are themselves licensed by Th cells to directly activate CD8⁺ T cells (FIG. 1B) (5) offers the advantage that a single licensed APC can contact multiple CD8⁺ T cells, and thereby expand the activation signal. However, a very limited number of DCs arriving in lymph nodes would interact with many CD4⁺ T cells, and the evidence demonstrates that they both induce marked proliferative responses among the naïve Ag-specific CD4⁺ T cell population, and also bestow on them of these progeny Th-APC functionality. In turn, each new Th-APC can interact with and activate naïve CD8⁺ CTL precursor cells, such that they also undergo expansion. The gain in this system is thereby dramatically increased even before the newly activated CTL precursors begin to proliferate. The discovery of the inventor also fits in well with the practical and theoretical constraints of Th-cell-dependent CTL responses in the host. Experimental evidence clearly shows that provision of IL-2 dramatically augments the efficiency of precursor CTL expansion (2-4). The inventor has shown that Th-APCs produce IL-2, and the data explains how CD4⁺ Th cells' IL-2 would be efficiently and precisely targeted to Ag-specific CD8⁺ T cells. It also addresses the requirement for cognate CD4⁺ T cell help for CD8⁺ CTL precursors (3,4,61), with the APCs in this case being by definition a cognate T helper cell.

Taken together, this study clearly delineates the role CD4⁺ Th-APCs can play in stimulation of CD8⁺ CTL responses. It also provides a solid experimental foundation for each of the tenants of a new dynamic model of sequential two-cell interactions by CD4⁺ Th-APCs in Th-cell-dependent CTL immune responses. Not only are Th-APC effective inducers of Ag-specific CTL activity in vitro, but also they efficiently induce protective anti-tumor immunity in vivo, thereby confirming their physiological relevance. While the inventor has addressed multiple parameters of this new model in the context of Th-cell-dependent CTL responses, in principle its conditions could be equally well met in regulatory T cell-dependent tolerance induction. Thus, T helper-antigen presenting cells can be used in antitumor immunity, cancer vaccine development and other immune disorders (e.g., autoimmunity).

### Example 2

### Targeting CD4⁺ T Cells with Exosomes

Materials and Methods

Reagents, Cell Lines and Animals

Ovalbumin (OVA) was obtained from Sigma (St. Louis, Mo.). OVA I (SIINFEKL) and OVA II (ISQAVHMHAEINEAGR), which are OVA peptides specific for H-2K^{b} and Ia^{b}, respectively (33,32). Mut I (FEQNTAQP) peptide is specific for H-2K^{b} of an irrelevant 3LL lung carcinoma. All peptides were synthesized by Multiple Peptide Systems (San Diego, Calif.). Biotin-labeled or fluorenscein isothiocyanate (FITC)-labeled antibodies (Abs) specific for H-2K^{b} (AF6-88.5), Ia^{b} (AF6-120.1), CD3 (145-2C11), CD4 (GK1.5), CD8 (53-6.7), CD11c (HL3), CD25 (7D4), CD40 (IC10), CD44 (IM7), CD54 (3E2), CD62L (MEL-14), CD69 (H1.2F3), CD80 (16-10A1), IL-7R (4G3) and Vα2Vβ5⁺ TCR (MR9-4) as well as FITC-conjugated avidin were all obtained from Pharmingen Inc. (Mississauga, Ontario, Canada). The anti-H-2K^{b}/OVA I complex (PMHC I) Ab was obtained from Dr. Germain (National Institute of Health, Bethesda, Md.) (62). The anti-LFA-1, interleukin (IL)-2, interferon (IFN)-γ and tumor necrosis factor (TNF)-α Abs, the cytotoxic T lymphocyte-associated Ag (CTLA4/Ig) fusion protein, the recombinant mouse IL-4 and granulocyte-macrophage colony-stimulating factor (GM-CSF) were purchased from R&D Systems Inc (Minneapolis, Minn.). The 5-carboxy-fluorescein diacetate succinimidyl ester (CFSE) was obtained from Molecular Probes, Eugene, Oreg. The mouse thymoma cell line EL4 and OVA-transfected EL4 (EG7) cell line were obtained from American Type Culture Collection (ATCC). The highly lung metastatic BL/6-10 and the OVA-transfected BL6-10 (BL6-10_{OVA}) melanoma cell lines were generated in the inventor's own laboratory (63). Female C57BL/6 (B6, CD45.2+) (32), C57BL/6.1 (B6.1, CD45.1⁺), OVA-specific TCR-transgenic OT I and OT II mice, and H-2K^{b}, Ia^{b}, IL-2, IFN-γ, TNF-α, CD54 and CD80 gene knockout (KO) mice on a C57BL/6 background were obtained from the Jackson Laboratory (Bar Harbor, Mass.). Homozygous OT II/H-2K^{b-/-}, OT II/CD54^{-/-}, OT II/CD80^{-/-}, OT II/IL-2^{-/-}, OT II/IFN-γ and OT II/TNF-α^{-/-} mice were generated by backcrossing the designated gene KO mice onto the OT II background for three generations. Rat insulin promoter (RIP)-mOVA mice that are on C57BL/6 background were obtained from The Walter and Eliza Hall Institute of Medical Research (Melbourne, Australia). They express OVA under the RIP and have, as such, OVA as a neo-self-antigen. They are transgenic for truncated OVA that is expressed as membrane bound molecule in pancreatic islets, kidney proximal tubules, and testis of male mice. All mice were treated according to animal care committee guidelines of the University of Saskatchewan.

DC Generation

Mouse spleen DCs were generated as described previously (47). Briefly, spleen cells were prepared in PBS with 5 mM EDTA, washed, and incubated in culture medium with 7% FCS at 37° C. for 2 hr. Nonadherent cells were removed by gentle pipetting with warm serum free medium. Adherent cells were cultured overnight in medium with 1% normal mouse serum, GM-CSF (1 ng/ml) and OVA (0.2 mg/ml). These DCs were termed as DC_{OVA}. DC generated from H-2 K^{b}, CD54 and CD80 gene KO mice were referred to as (K^{b-/-})DC_{OVA}, (CD54^{-/-})DC_{OVA} and (CD80^{-/-})DC_{OVA}, respectively.

Exosome Preparation

Exosomes (EXO) preparation and purification as described previously (64,65). Briefly, culture supernatants of OVA-pulsed bone marrow-derived DC (66) were subjected to four successive centrifugations at 300×g for 5 min to remove cells, 1,200×g for 20 min and 10,000×g 30 min to remove cellular debris and 100,000×g for 1 h to pellet EXO. The EXO pellets were washed twice in a large volume of PBS and recovered by centrifugation at 100,000×g for 1 h. The amount of exosomal proteins recovered was measured by Bradford assay (Bio-Rad, Richmond, Calif.). EXO derived from DC_{OVA} of wild-type C57BL/6 and C57BL/6.1 was termed as EXO_{OVA} and EXO_{6.1}, respectively. To generate CFSE-labeled EXO, DC were stained with 0.5 μM CFSE at 37° C. for 20 minutes (32) and washed three times with PBS, and then pulsed with OVA protein in AIM-V serum-free medium for overnight. The CFSE-labeled EXO (EXO_{CFSE}) were harvested and purified from the culture supernatants as described above.

CD4⁺ T Cell Preparation

Naïve OVA-specific T (nT) cells were isolated from OVA-specific TCR transgenic OT I and OT II mouse spleens, enriched by passage through nylon wool columns, and then purified by negative selection using anti-mouse CD8(Ly2) or CD4 (L3T4) paramagnetic beads (DYNAL Inc) to yield populations that were >98% CD4⁺/Vα2Vβ5⁺ or CD8⁺/Vα2Vβ5⁺, respectively (63). To generate active OT II CD4⁺ T cells, the spleen cells from OT II mouse were cultured in RPMI1640 medium containing IL-2 (20 U/ml) and Con A (1 μg/ml) for 3 days (23). The Con A-activated CD4⁺ T (aT) cells were then purified as described above.

Exosomal Molecule Uptake by CD4⁺ T Cells

Firstly, the CD4⁺ nT and aT cells were incubated with EXO_{CFSE} (10 μg/1×10⁶ T cells) at 37° C. for 4 hours and then analyzed for CFSE staining by flow cytometry (66). In another set of experiment, the CD4⁺ nT and aT cells were co-cultured with EXO_{6.1} and then analyzed for expression of CD45.1 molecule. To further determine the transfer of exosomal molecules to T cells, the CD4⁺ nT and aT cells from OT II mice or OT II mice with different gene KO were incubated with EXO_{OVA}, and then analyzed for expression of H-2 K^{b}, CD54, CD80 and pMHC I by flow cytometry. For blocking assays, CD4⁺ T cells from H-2K^{b} gene KO mice were incubated with anti-H-2 Kb and anti-Ia^{b} Abs (12 μg/ml) or CTLA-4/1 g (12 μg/ml), respectively, on ice for 30 min, then were co-cultured with EXO_{OVA} for 4 h at 37° C. The cells were harvested and analyzed for expression of H-2K^{b} by flow cytometry. The CD4⁺ nT and aT cells co-cultured with EXO_{OVA} were termed nT_{EXO} and aT_{EXO}, respectively. The CD4⁺ aT cells from mice with H-2K^{b}, CD54, CD80, IL-2, IFN-γ and TNF-α gene KO, which were previously co-cultured with EXO_{OVA}, were termed CD4⁺ aT_{EXO}(K^{b-/-}), aT_{EXO}(CD54^{-/-}), aT_{EXO}(CD80^{-/-}), aT_{EXO}(IL-2^{-/-}), aT_{EXO}(IFN-γ^{-/-}) and aT_{EXO}(TNF-α^{-/-}) cells, respectively. The cytokine profiles of aT_{EXO}(K^{b-/-}), aT_{EXO}(CD54^{-/-}) and aT_{EXO}(CD80^{-/-}) cells are similar to that of aT_{EXO} cells, whereas the cytokine profiles of aT_{EXO(IL-}2^{-/-}), aT_{EXO}(IFN-γ^{-/-}) and aT_{EXO}(TNF-α^{-/-}) cells are also similar to that of aT_{EXO} cells except for the specific cytokine (IL-2 or IFN-γ or TNF-α) deficiency.

T Cell Proliferation Assay

To assess the functional effect of CD4⁺ nT_{EXO} and aT_{EXO} cells, a CD8⁺ T cell proliferation assay was performed. The CD4⁺ nT_{EXO} and aT_{EXO} (0.3×10⁵ cells/well) cells and their 2-fold dilutions were cultured with a constant number of naïve OT I CD8⁺ T cells (1×10⁵ cells/well) in presence or absence of CD4⁺ CD25+T cells (0.3×10⁵ cells/well) purified from C57BL/6 mouse spleen T cells using CD25-microbeads (Miltenyi Biotech, Auburn, Calif.). To examine the molecular mechanism, a panel of reagents including anti-H-2K^{b}, I-A^{b} and LFA-1 Abs and CTLA-4/Ig fusion protein (each 10 μg/ml), a mixture of the above reagents (as mixed reagents) and a mixture of isotype-matched irrelevant Abs (as control reagents) were added to the cell cultures, respectively. In another set of experiments, C57BL/6 and RIP-mOVA mice were s.c. immunized with OVA II peptide (500 μM) emulsified 1:1 (v/v) in CFA (50 μl/each mouse). Ten days after immunization, single cell suspensions were prepared from the regional lymph nodes of immunized mice. Serial dilutions of OVA II peptides were mixed with 5×10⁵ cells per well in microtiter plates in RPIMI 1640 containing 5% syngenic mouse serum. After culturing for 3 days, thymidine incorporation was determined by liquid scintillation counting (34).

Tetramer Staining Assay

C57BL/6 mice were i.v. injected with irradiated (4,000 rad) DC_{OVA}, nT_{EXO} and aT_{EXO} cells (3×10⁶ cells), respectively. In one set of experiments, one hundred microliter of blood was taken from the tail of the above mice 6 days after immunization. The blood samples were incubated with PE-conjugated H-2K^{b}/OVA₂₅₇₋₂₆₄ tetramer (Beckman Coulter, Mississauga, Ontario, Canada) and FITC-conjugated anti-CD8 Ab for 30 min at room temperature. The erythrocytes were then lysed using lysis/fixed buffer (Beckman Coulter). The cells were washed and analyzed by flow cytometry. Three months after the immunization, the mouse tail blood was analyzed using PE-conjugated tetramer, and ECD-conjugated anti-CD44 and FITC-conjugated anti-CD8 Abs for detection of OVA-specific CD8⁺ Tm cells by flow cytometry. In another set of experiments, the above immunized mice were i.v. boosted with irradiated DC_{OVA} (0.5×10⁶) three months after immunization. The blood samples obtained from these mice 4 days after the boost were analyzed for OVA-specific CD8⁺ Tm cell expansion by flow cytometry.

Cytotoxicity Assay

In vivo cytotoxicity assays were performed as previously described (63). Briefly, C57BLU6 mice were i.v. immunized with above cells, respectively. Splenocytes were harvested from naïve mouse spleens and incubated with either high (3.0 μM, CFSE^{high}) or low (0.6 μM, CFSE^{low}) concentrations of CFSE, to generate differentially labeled target cells. The CFSE^{high} cells were pulsed with OVA I peptide, whereas the CFSE^{low} cells were pulsed with Mut 1 peptide and served as internal controls. These peptide-pulsed target cells were washed extensively to remove free peptides, and then i.v. co-injected at 1:1 ratio into the above immunized mice six days after immunization. Sixteen hrs after the target cell delivery, the spleens of immunized mice were removed and residual CFSE^{high} and CFSE^{low} target cells remaining in the recipients' spleens were analyzed by flow cytometry.

Animal Studies

To examine the antitumor protective immunity conferred by EXO-targeted CD4⁺ T cells wild-type C57BL/6, Ia^{b} or K^{b} KO mice (n=8) lacking CD4⁺ or CD8⁺ T cells were injected i.v. with irradiated (4,000 rad) DC_{OVA}, nT_{EXO} and aT_{EXO} cells or aT_{EXO} cells (1×10 ⁶ cells/mouse) with various gene KO, respectively. The mice injected with PBS as a control. In one set of experiments, wild-type C57BL/6 mice were immunized with irradiated (4,000 rad) aT_{EXO} cells (1×10⁶ cells/mouse) with various gene KO. The immunized mice were challenged i.v. with 0.5×10⁶ BL6-10_{OVA} or BL6-10 cells six days subsequent to the immunization to assess antitumor immunity. In another set of experiments, wild-type C57BL/6 mice were immunized with irradiated (4,000 rad) DC_{OVA} and aT_{EXO} cells (1×10⁶ cells/mouse). The immunized mice were then challenged i.v. with 2×10⁶ BL6-10_{OVA} cells three months subsequent to the immunization to assess development of tumor-specific memory T (Tm) cells. The mice were sacrificed 4 weeks after tumor cell injection, and the lung metastatic tumor colonies were counted in a blind fashion. Metastases on freshly isolated lungs appeared as discrete black pigmented foci that were easily distinguishable from normal lung tissues and confirmed by histological examination. Metastatic foci too numerous to count were assigned an arbitrary value of >100 (63).

Results

CD4⁺ T Cells Uptake EXO in Both Ag-Specific and None-Specific Manners

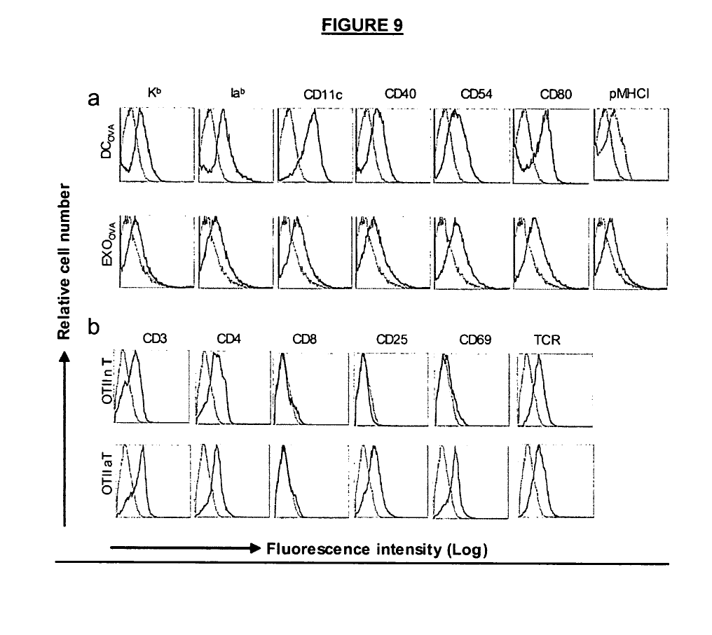

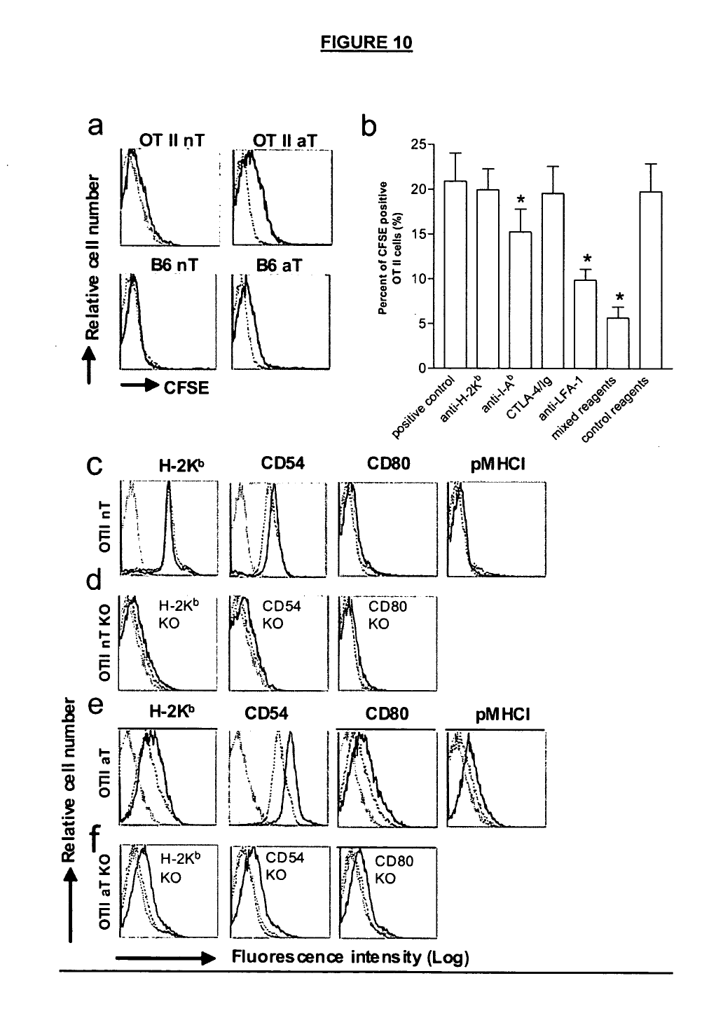

Similar to OVA-pulsed DC_{OVA}, MHC class I (Kb) and class II (Ia^{b}), CD11c, CD40, CD54, CD80 and PMHC I complex were detected on DC_{OVA}-derived EXO_{OVA}, but with a less content compared with DC_{OVA} (FIG. 9a). The naïve CD4⁺ T (nT) and Con A-stimulated active CD4⁺ T (aT) cells derived from transgenic OT II mice expressed both CD4 and TCR molecules (FIG. 9b). The CD4⁺ aT cells expressing active T cell markers (CD25 and CD69), but not the CD4⁺ nT cells, secreted IL-2 (−2.4 ng/ml per 10⁶ cells/24 hr), IFN-γ (˜2.0 ng/ml per 10⁶ cells/24 hr) and TNF-α (˜1.7 ng/ml per 10⁶ cells/24 hr), but no IL-4 and IL-10, indicating that they are type 1 helper T cells. To assess EXO uptake by T cells, CD4⁺ nT and aT cells derived from OT II and wild-type C57BL/6 (B6) mice were incubated with CFSE-labeled EXO (EXO_{CFSE}), and then analyzed by flow cytometry. As shown in FIG. 10a, the CFSE dye was detectable on OT II CD4⁺ nT and aT cells as well as B6 CD4⁺ aT cells, but not on B6 CD4⁺ nT cells. To elucidate the molecular mechanisms involved in EXO uptake, a panel of reagents was then used in blocking assay. As shown in FIG. 10b, the anti-Ia^{b} and LFA-1 Abs, but not the CTLA-4/Ig fusion protein and anti-H-2K^{b} Ab, were able to block EXO uptake, indicating that the EXO uptake by CD4⁺ T cells is mediated by both OVA-specific Ia^{b}/TCR and non-specific CD54/LFA-1 interactions, which is consistent with the previous reports (20,67).

CD4⁺ T Cells Acquire pMHC I and Costimulatory Molecules by EXO Uptake

Similar to the above transferred CFSE dye, other EXO molecules such as MHC class I and II, CD54 and CD80 molecules were transferred onto OT II CD4⁺ nT and aT cells (FIGS. 10c and 10e). In addition, pMHC I complexes, the critical components in stimulation of OVA-specific CD8⁺ CTL responses, were also transferred onto the CD4⁺ T cells. Since the original CD4⁺ T cells, especially CD4⁺ aT cells expressed some of the above exosomal molecules, it was necessary to confirm that an increased expression of these molecules is not due to their endogenous up-regulation. Thus, OT II CD4⁺ T cells were incubated with different gene KO with EXO, and then analyzed by flow cytometry. As shown in FIGS. 10d and 10f, the original OT II CD4⁺ nT and aT cells with gene KO did not express endogenous H-2 K^{b}, CD54 and CD80, respectively. However, after uptake of EXO_{OVA}, each of them did display their exogenous H-2 K^{b}, CD54 and CD80 molecules, indicating that an increased expression of the above molecules on CD4⁺ T cells is due to an uptake of EXO molecules.

EXO-Targeted CD4⁺ T Cells Stimulate Naïve CD8⁺ T Cell Proliferation in Presence of CD4⁺ CD25+Tr Cells In Vitro

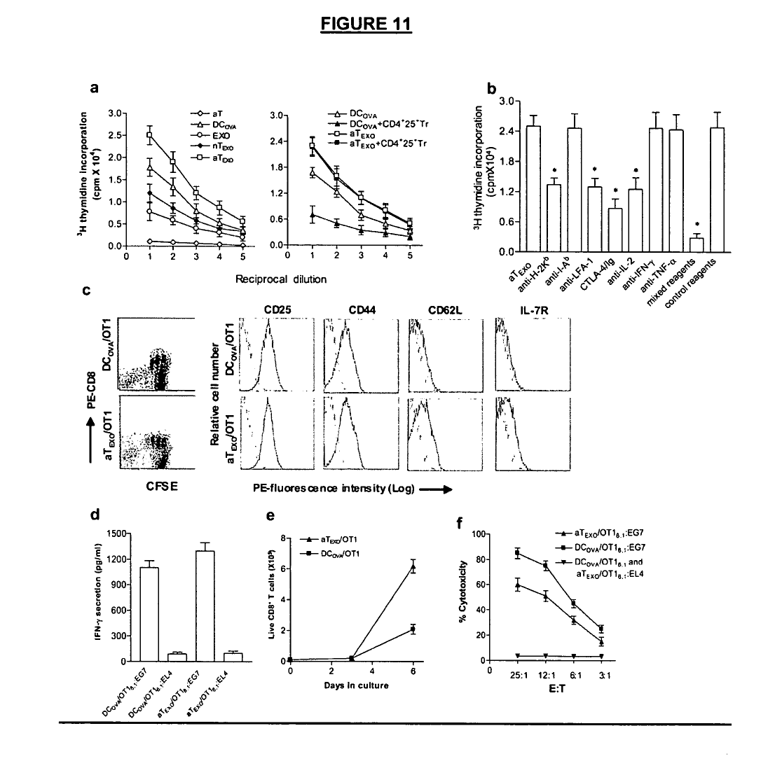

The stimulatory effect of EXO-targeted CD4⁺ T cells was then examined. As shown in FIG. 11a, EXO_{OVA} could stimulate CD8⁺ T cell proliferation in vitro, which is consistent with a previous report by Hwang et al (20), but in a much less extent compared with DC_{OVA}. However, EXO-targeted active aT_{EXO} is a stronger stimulator in CD8⁺ T cell proliferation than DC_{OVA}, whereas naïve nT_{EXO} is a relatively weak stimulator. CD4⁺ CD25⁺ Tr cells inhibited DC_{OVA}-stimulated CD8⁺ T cell proliferation. However, aT_{EXO} maintained its stimulatory effect in presence of CD4⁺ CD25⁺ Tr cells, indicating that aT_{EXO} may bypass CD4⁺ CD25⁺ Tr cell-mediated suppressive pathways. To investigate the molecular mechanism involved in CD8⁺ T cell proliferation, a panel of reagents were added to the cell cultures. As shown in FIG. 11b, anti-H-2K^{b}, anti-LFA-1, anti-IL-2 Abs, and CTLA-4/Ig, but not anti-Ia^{b}, anti-IFN-γ and anti-TNF-α Abs, significantly inhibited CD8⁺ T cell proliferative responses in the co-cultures by 49%, 52%, 62% and 49% (p<0.05), respectively, indicating that the CD8⁺ T cell proliferation is critically dependent on OVA-specific pMHC I/TCR interaction, and greatly affected by non-specific costimulations (CD80/CD28 and CD54/LFA-1).

EXO-Targeted CD4⁺ T Cells Stimulate Naïve CD8⁺ T Cell Differentiation into Central Memory T Cells In Vitro

A phenotypic characterization of the above in vitro aT_{EXO}-primed CD8⁺ T cells was then conducted. The data showed that both DC_{OVA} and aT_{EXO} priming resulted in several cycles of CD8⁺ CFSE-T cell division, and the primed T cells displayed the expression of CD25, CD44 (Tm marker) (68) and CD62L. However, aT_{EXO}-primed CD8⁺ T cells displayed IL-7R and higher CD62L expression than DC_{OVA}-primed ones with no IL-7R expression (FIG. 11c), indicating they may be prone to becoming long-lived Tm cells. It was then examined whether aT_{EXO}-primed CTL exhibited any other functional traits attributed to typical memory cells. These traits include (i) secretion of IFN-γ upon Ag stimulation, (ii) the enhanced survival and proliferation in response to IL-7 and IL-15 (69), and (iii) the capacity to generate Ag-specific CTL. The data also showed that both DC_{OVA}- and aT_{EXO}-primed CD8⁺ T cells secrete IFN-γ upon Ag stimulation by EG7 tumor cells (FIG. 11d). However, aT_{EXO}-primed CTL expanded better in presence of IL-2, IL-7 and IL-15 than DC_{OVA}-primed ones (FIG. 11e). In chromium release assay, aT_{EXO}-primed CTL (aT_{EXO}/OT I_{6.1}) showed cytotoxicity to OVA-expressing EG7 tumor cells, but at a relatively lower level than DC_{OVA}-primed ones (DC_{OVA}/OT I_{6.1}) (FIG. 11f). Taken together, the inventor's results indicate that DC_{OVA}-primed CD44⁺CD62L^{low}IL-7R⁻ and aT_{EXO}-primed CD44⁺CD62L^{high}LL-7R⁺ CTL, which have high and low cytotoxicity to tumor cells, are consistent with typical effector and central memory CTL (emCTL and cmCTL), respectively (70,71).

EXO-Targeted CD4⁺ T Cells Activate CD4⁺ T Cell-Independent CD8⁺ T Cell Proliferation in Wild-Type C57BL/6 Mice In Vivo

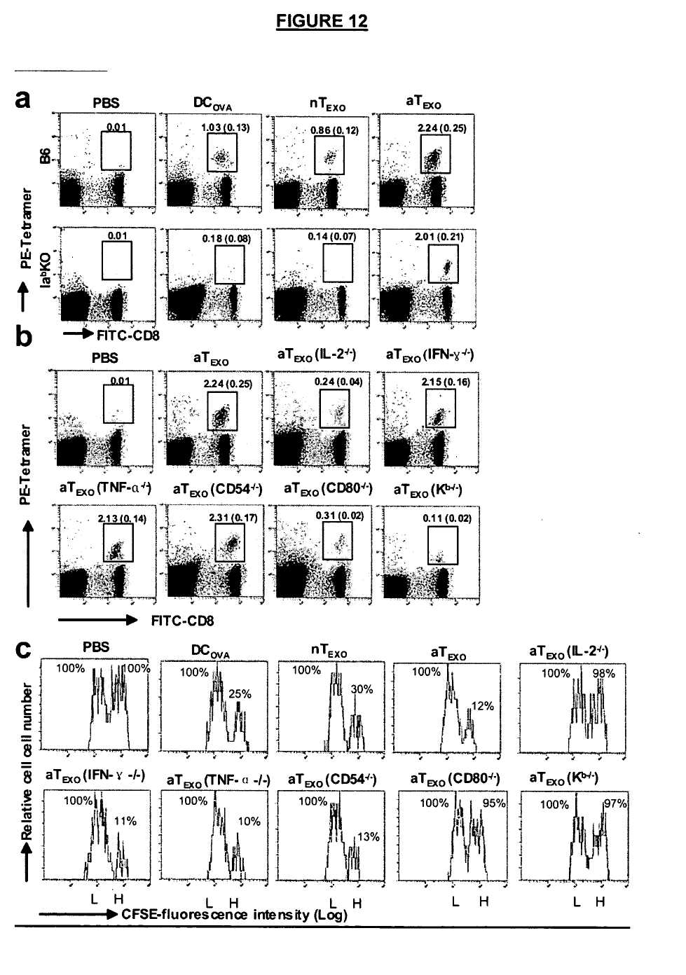

A tetramer staining assay was then performed to detect OVA-specific CD8⁺ T cells in wild-type or MHC class II (Ia^{b}) gene KO mice 6 days after immunizations with DC_{OVA}, aT_{EXO} and nT_{EXO} cells, respectively. As shown in FIG. 12a, DC_{OVA}, aT_{EXO} and nT_{EXO} cells stimulated proliferation of H-2K^{b}/OVA₂₅₇₋₂₆₄ tetramer-positive CD8⁺ T cells accounting for 1.03%, 2.24% and 0.86% of the total spleen CD8⁺ T cells in wild-type C57BL/6 (B6) mice, respectively, indicating that EXO-targeted aT_{EXO} is the strongest stimulator among the three. In lab gene KO mice lacking CD4⁺ T cells, however, only aT_{EXO}, but not DC_{OVA} and nT_{EXO}, could still stimulate OVA-specific CD8⁺ T cell responses (2.01%), indicating that the aT_{EXO}-induced CD8⁺ T cell response is CD4⁺ T cell independent, whereas those of DC_{OVA} and nT_{EXO} are CD4⁺ T cell dependent.

The Stimulatory Effect of EXO-Targeted CD4⁺ T Cells is Mediated by its IL-2 and Acquired CD80 Costimulation and Specifically Delivered to CD8⁺ T Cells In Vivo Via Acquired pMHC I

By using aT_{EXO} with different gene KO, the stimulation of OVA-specific CD8⁺ T cell responses by aT_{EXO}(IL-2^{-/-}) (0.24%) and aT_{EXO}(CD80^{-/-}) (0.31%) cells, but not with aT_{EXO}(IFN-γ^{-/-}) (2.15%), aT_{EXO}(TNF-α^{-/-}) (2.13%) and aT_{EXO}(CD54^{-/-}) (2.31%) cells, was almost lost (FIG. 12b), indicating that the stimulatory effect of aT_{EXO} is mediated by its IL-2 and acquired CD80 costimulation. Interestingly, aT_{EXO}(K^{b-/-}) cells (0.11%) with similar cytokine profile as aT_{EXO} (data not shown), but without acquired pMHC I complexes, also completely lost their stimulatory effect, indicating that the stimulatory effect of aT_{EXO} is specifically delivered to CD8⁺ T cells in vivo via acquired exosomal pMHC I complexes.

EXO-Targeted CD4⁺ T Cells Stimulate CD8⁺ T Cell Differentiation into CTL Effectors in Wild-Type C57BL/6 Mice In Vivo

To assess aT_{EXO}-induced CD8⁺ T cell differentiation into CTL, OVAI peptide-pulsed splenocytes that had been strongly labeled with CFSE (CFSE^{high}) were adoptively transferred, as well as the control peptide Mut1-pulsed splenocytes that had been weakly labeled with CFSE (CFSE^{low}), into the recipient mice that had been vaccinated with DC_{OVA}, aT_{EXO} and nT_{EXO} cells, respectively. As expected, the mice immunized with aT_{EXO} had the largest loss of the CFSE^{high} (OVAI peptide-pulsed) cells among the three stimulators [DC_{OVA} (75%), aT_{EXO} (88%) and nT_{EXO} (70%)] (FIG. 12c), indicating that aT_{EXO} can most efficiently stimulate CD8⁺ T cell differentiation into CTL effectors. Interestingly, the aT_{EXO}-induced cytotoxicity was substantially lost in aT_{EXO}(IL-2^{-/-})-(2%) and aT_{EXO}(CD80^{-/-})-immunized (5%) mice, but not in aT_{EXO}(IFN-γ^{-/-})-(89%), aT_{EXO}(TNF-α^{-/-})-(90%) and aT_{EXO}(CD54^{-/-})-immunized (87%) ones, thus further confirming that aT_{EXO}'s stimulatory effect is mediated by its IL-2 secretion and acquired CD80 costimulation. In addition, the aT_{EXO}(K^{b-/-})-vaccinated mice did not display any killing activity (3%), again confirming that the acquired pMHC I complexes play a critical role in targeting CD4⁺ aT_{EXO}'s stimulatory effect to OVA-specific CD8⁺ T cells in vivo.

EXO-Targeted CD4⁺ T Cells Breaks Immune Tolerance in RIP-mOVA Transgenic Mice

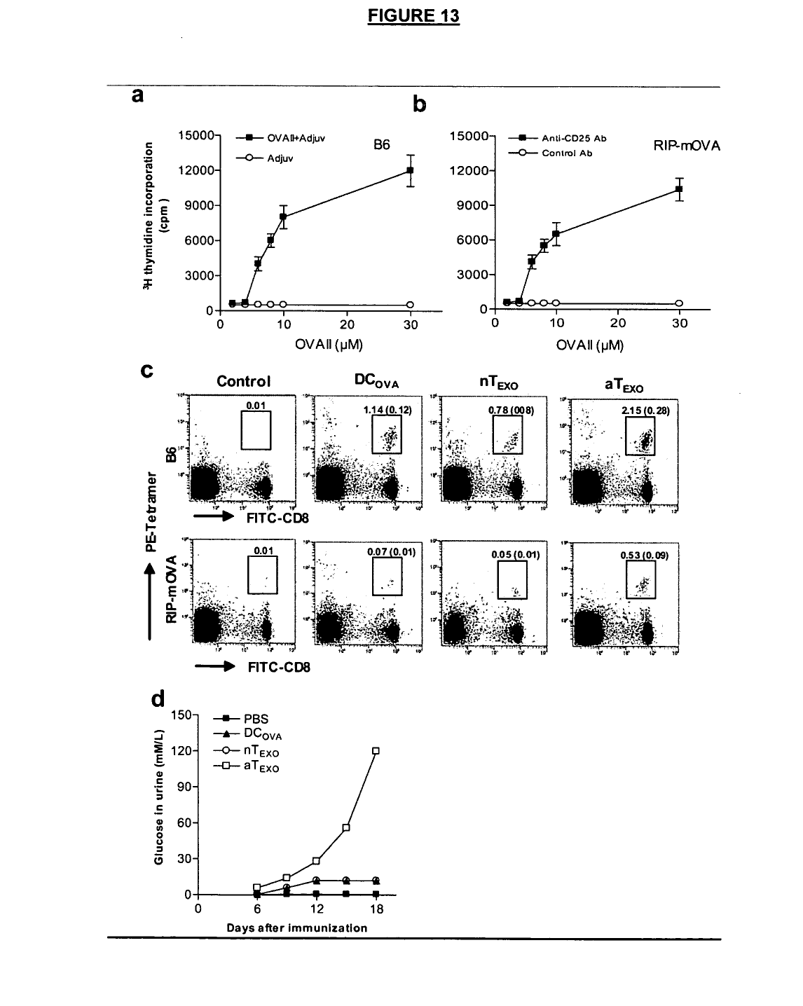

RIP-mOVA transgenic mice expressing self-OVA exhibited deletional tolerance mediated by autoreactive CD8⁺ T cells (72). Wild-type C57BL/6 (B6) and RIP-mOVA transgenic mice were s.c. immunized with OVAII peptide in CFA. The data demonstrated that the lymph node T cells from immunized B6 mice responded normally to OVA II peptide, whereas those from immunized RIP-mOVA mice did not proliferate in presence of OVAII peptide stimulation (FIG. 13a). Interestingly, when RIP-mOVA mice had been previously treated with anti-CD25 Ab to delete CD4⁺CD25⁺ Tr cells (73) before immunization, lymph node T cells resumed their normal responses to OVAII stimuli (FIG. 13b), indicating the exist of CD4⁺ Tr cell-mediated OVA-specific immune tolerance in RIP-mOVA mice, which is consistent with a previous report (74). To assess the potential breakage of immune tolerance, B6 and RIP-mOVA mice were immunized with DC_{OVA}, aT_{EXO} and nT_{EXO} cells, respectively. As shown in FIG. 13c, DC_{OVA}, aT_{EXO} and nT_{EXO} cells stimulated tetramer-positive CD8⁺ T cell responses accounting for 1.14%, 2.15% and 0.78% of the total spleen CD8⁺ T cells in wild-type B6 mice, respectively. However, only aT_{EXO}, but not DC_{OVA} and nT_{EXO}, still stimulated 0.53% tetramer-positive CD8⁺ T cell responses, indicating that EXO-targeted active CD4⁺ T (aT_{EXO}) cells can break immune tolerance in RIP-mOVA transgenic mice. This was further confirmed by the animal diabetes studies. Again, only aT_{EXO}, but not DC_{OVA} and nT_{EXO} cells, induced diabetes in all 8/8 RIP-mOVA mice (FIG. 13d).

EXO-Targeted CD4⁺ T Cells Induce Strong Antitumor Immunity in Wild-Type C57BL/6 Mice

As shown in Exp I of Table 2, all the mice injected with PBS had large numbers (>100) of lung metastatic tumor colonies. The aT_{EXO} vaccine induced a complete immune protection against BL6-10_{OVA} tumor cell challenge (0.5×10⁶ cells/mouse) in 8/8 (100%), whereas both DC_{OVA} and nT_{EXO} cell vaccines only protected 6/8 (75%) and 5/8 (63%) mice, respectively, indicating that CD4⁺ aT_{EXO} induce stronger antitumor immunity than DC_{OVA}. The specificity of the protection was confirmed with the observation that aT_{EXO} did not protect against BL6-10 tumors that did not express OVA, with all mice having large numbers (>100) of lung metastatic tumor colonies. To study the immune mechanism, Ia^{b} and H-2K^{b} gene KO mice were used for immunization of aT_{EXO} cells. As shown in Exp II of Table 2, most of Ia^{b} gene KO (7/8) mice lacking CD4⁺ T cells were still tumor free. However, all H-2K^{b} gene KO mice (8/8) lacking CD8⁺ T cells had numerous lung tumor metastases, confirming that aT_{EXO}-induced antitumor immunity is CD4⁺ Th cell independent.

EXO-Targeted CD4⁺ T Cell's Stimulatory Effect is Mediated by IL-2 Secretion and Acquired CD80 Costimulation, and Specifically Delivered to CD8⁺ T Cells In Vivo Via Acquired pMHC I

To elucidate the molecular mechanism, aT_{EXO} cells with respective gene deficiency were used for immunizations. It was found that aT_{EXO}(IFN-γ^{-/-})-, aT_{EXO}(TNF-α^{-/-})- and aT_{EXO}(CD54^{-/-})-immunized mice (8/8) had no lung tumor metastases, whereas aT_{EXO}(IL-2^{-/-})-(7/8) and aT_{EXO}(CD80^{-/-})-immunized (5/8) mice lost their antitumor immunity (Exp III of Table 2), indicating that aT_{EXO}-secreted IL-2 and acquired CD80 costimulation, but not IFN-γ, TNF-α and acquired CD54, play an important role in stimulation of CD8⁺ CTL responses in vivo, which is consistent with the above data (FIG. 12). Interestingly, most (7/8) of mice immunized with aT_{EXO}(pMHC I^{-/-}) without acquired pMHC I had large numbers (>100) of lung tumor colonies, indicating that the above aT_{EXO} cell's stimulatory effect is specifically delivered to CD8⁺ T cells in vivo via acquired pMHC I complexes.

EXO-Targeted CD4⁺ T Cells Induce Efficient Long-Term OVA-Specific CD8⁺ T Cell Memory

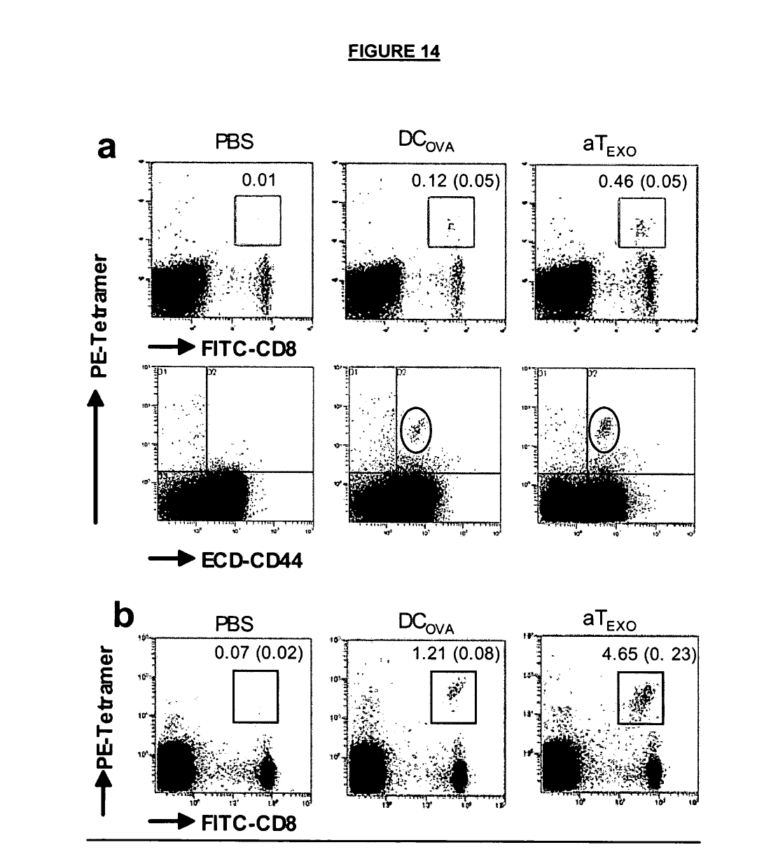

Active CD8⁺ T cells can become long-lived memory T (Tm) cells after adoptive transfer in vivo (75). These aT_{EXO}-activated CD8⁺ T cells were then assessed whether they can also become long-lived Tm cells. As shown in FIG. 14a, 0.12%, and 0.46% OVA-specific CD8⁺ T cells were detected in peripheral blood of immunized mice three months after the immunization. These OVA-specific CD8⁺ T cells were also CD44 (Tm marker) (68) positive, indicating that they are OVA-specific CD8⁺ Tm cells. In addition, the survived aT_{EXO}-stimulated CD8⁺ Tm cells are nearly 4-fold compared with the survived DC_{OVA}-stimulated ones, further confirming that aT_{EXO}-primed CD44⁺CD62L^{high}IL-7R⁺ CTL with low cytotoxicity to tumor cells are long survival cmCTL. The recall responses were assessed on day 4 after the boost of immunized mice with DC_{OVA}. As shown in FIG. 14b, there were few OVA-specific CD8⁺ T cells detected in peripheral blood of the PBS control mice, indicating that the primary proliferation of OVA-specific CD8⁺ T cells derived from DC_{OVA} boost is almost undetectable in at that time point. As expected, CD8⁺ Tm cells were expanded by 10 folds in these immunized mice after the boost, indicating that these CD8⁺ Tm cells are functional. In another set of experiments, the above immunized mice were challenged with a high dose (2×10⁶ cells per mouse) of BL6-10_{OVA} tumor cells. Only 4/8 (50%) of mice immunized with DC_{OVA} were tumor free, whereas all 8/8 (100%) of mice immunized with aT_{EXO} did not have any lung metastasis (Exp. III of Table 2), indicating that EXO-targeted CD4⁺ T cells can induce more efficient long-term CD8⁺ T cell memory than DC_{OVA}.

Discussion

According to the progressive linear differentiation hypothesis (76), T cell differentiation involves a phase of proliferation preceding the acquisition of fitness and effector function. Primed CD8⁺ T cells reach a variety of differentiation stages that contain effector cells as well as cells that have been arrested at intermediate levels of differentiation. Thus, they retain a flexible gene imprinting. T cells that may survive after retraction phase of an immune response can be resolved into distinct subsets of either central memory CTL (cmCTL) cells representing cells at intermediate levels of differentiation or fully differentiated effector memory CTL (emCTL) cells with effector capacity (77,78). It has been shown that a strong Ag presentation stimulates development of effector CTL, whereas a less efficient Ag presentation can lead to the generation of central memory CTL (79). In this study, the inventor demonstrated that CD4⁺ aT_{EXO} cells were able to stimulate naïve CD8⁺ T cell differentiation into central memory CD44+CD62^{high}IL-7R⁺ T cells with less cytotoxicity and longer survival capacity leading to strong memory T cell responses, compared with DC_{OVA}-primed CD44+CD62^{low}IL-7R⁻ effector memory CTL with high cytotoxicity and shorter survival capacity in vivo.

CD4⁺ CD25⁺ regulatory T (Tr) cells develop in the thymus and then enter the peripheral tissues, where they suppress activation of other self-reactive T cells (73,80). It has been reported that an elevated number of CD4⁺CD25⁺ Tr cells was detected in tumors (69,81), which suppressed the anti-tumor immune responses by inhibition of naïve CD4⁺ T cell proliferation and CD4⁺ T cell helper effect (82-84) as well as DC maturation (85). Therefore, how to combat immune tolerance becomes a critical challenge in cancer immunotherapy (1). In this study, for the first time, it was demonstrated that EXO-targeted CD4⁺ aT_{EXO} cells, but not DC_{OVA}, can stimulate CD8⁺ T cell proliferation in presence of CD4⁺CD25⁺ Tr cells in vitro and RIP-mOVA transgenic mice in vivo leading to development of OVA-specific cytotoxic T lymphocyte (CTL)-mediated diabetes. These results clearly indicate that EXO-targeted CD4⁺ aT_{EXO} cells can break CD4⁺ CD25+Tr cell-mediated immune tolerance, possibly due to its capacity of direct stimulation of CD8⁺ T cell responses in a CD4⁺ T helper cell- and DC-independent manner, thus bypassing the above CD4⁺ Tr cell-mediated suppressive pathways.

EXO-based vaccines have been shown to induce antitumor immunity (24-28). However, its efficiency was less effective because it only induced either prophylatic immunity in animal models (24-28) or very limited immune responses in clinical trials (86). The potential pathway of EXO-mediated immunity is through uptake of EXO by the host DC. In this study, DC_{OVA}-derived EXO were systemically characterized by flow cytometry. The inventor demonstrated that, in addition to the previously reported MHC class I and II and CD54 molecules, EXO also expressed CD11c and co-stimulatory molecule CD80. In addition, EXO also expressed MHC class I/OVA I peptide (PMHC I) complexes, the critical components in initiation of CD8⁺ CTL responses. The inventor also demonstrated that EXO itself can stimulate OT I CD8⁺ T cell proliferation in vitro, which is also consistent with a previous report by Hwang et al (87), but in a relatively mild fashion. Administration of attenuated T lymphocytes to animals has been shown to stimulate immune suppression and to prevent the development of experimental autoimmune diseases (88-90). Vaccination using myelin-basic-protein autoreactive T cells has also been applied to clinical trial in multiple sclerosis (91). Interestingly, for the first time, the inventor clearly showed that EXO-targeted CD4⁺ aT_{EXO} can more strongly stimulate OVA-specific immunogenic CD8⁺ CTL responses, antitumor immunity and CD8⁺ T cell memory in wild-type mice than EXO and DC_{OVA}. Furthermore, the inventor elucidated the molecular mechanisms involved in CD4⁺ aT_{EXO} cell vaccines by showing that (i) it is the IL-2 secretion and the acquired CD80 costimulation that mediate the CD4⁺ aT_{EXO} cell's stimulatory effect, and (ii) it is the acquired pMHC I complexes that play a critical role in targeting the stimulatory effect of CD4⁺ aT_{EXO} cells to CD8⁺ T cells in vivo.

Taken together, the inventor's data showed that OVA-pulsed DC (DC_{OVA})-derived EXO (EXO_{OVA}) can be uptaken by CD4⁺ T cells. EXO_{OVA}-uptaken (targeted) CD4⁺ T cells expressing acquired pMHC I and costimulatory CD80 molecules can break immune tolerance in RIP-mOVA transgenic mice, and induce OVA-specific central memory CD8⁺ T responses leading to more efficient antitumor immunity and CD8⁺ T cell memory in wild-type mice than DC_{OVA}. Therefore, the EXO-targeted CD4⁺ T cell vaccine may represent a new highly effective vaccine strategy for inducing immune responses against not only tumors, but also other infectious diseases.

### Example 3

### Targeting Dendritic Cells with Exosomes

Materials and Methods

Reagents, Cell Lines and Animals

Ovalbumin (OVA) protein was obtained from Sigma (St. Louis, Mo.). OVA I (SIINFEKL) peptide (33,32) and Mut I (FEQNTAQP) peptide specific for an irrelevant 3LL lung carcinoma (34) were synthesized by Multiple Peptide Systems (San Diego, Calif.). Biotin-labeled and fluorescein isothiocyanate (FITC)-labeled antibodies (Abs) specific for H-2K^{b} (AF6-88.5), Ia^{b} (AF6-120.1), CD4 (GK1.5), CD8 (53-6.7), CD11c (HL3), CD40 (IC10), CD54 (3E2), CD80 (16-10A1), CD44 (IM7), MyD88, CCR7 (4B12) and DC-specific ICAM-grabbing non-integrin (DC-SIGN) (5H-11) were obtained from Pharmingen Inc (Mississauga, Ontario, Canada). The anti-H-2 Kb/OVA I (PMHC I) complex Ab was obtained from Dr. Germain (National Institute of Health, Bethesda, Md.) (62). PE-labeled H-2K^{b}/OVA I tetramer Ab was obtained from Beckman Coulter (Mississauga, Ontario, Canada). Biotin-labeled Toll-like receptor (TLR)4 and TLR9 Abs were obtained from eBioscience (San Diego, USA). The anti-LFA-1, anti-K^{b}, anti-Ia^{b} and anti-DEC205 Abs, and the cytotoxic T lymphocyte-associated Ag (CTLA4/Ig) fusion protein, the recombinant mouse interleukin-4 (IL-4) and granulocyte-macrophage colony-stimulating factor (GM-CSF) were purchased from R&D Systems Inc (Minneapolis, Minn.). The cytochalasin D (CCD), D-mannose, D-glucose, D-fucose and D-glucosamine were purchased from SIGMA (St. Louis, Mo.). The 5-carboxy-fluorescein diacetate succinimidyl ester (CFSE) was obtained from Molecular Probes, Eugene, Oreg. The highly lung metastatic BL/6-10 and the OVA-transfected BL6-10 (BL6-10_{OVA}) melanoma cell lines were generated in the inventor's laboratory (63). The mouse EL4 and the OVA-transfected EL4 (EG7) thymoma cell lines were obtained from American Type Culture Collection (ATCC, Rockville, Md.). Female C57BL/6 (B6; CD45.2+), C57BL/6.1 (B6.1; CD45.1⁺), OVA-specific T cell receptor (TCR) transgenic OT I and OT II mice, and H-2 K^{b}, CD4, CD8, CD54 and CD80 gene knockout (KO) mice on a C57BL/6 background were all obtained from the Jackson Laboratory (Bar Harbor, Mass.). All mice were maintained in the animal facility at the Saskatoon Cancer Center and treated according to animal care committee guidelines of the University of Saskatchewan.

Generation of Bone Marrow-Derived DC

The generation of bone marrow (BM)-derived immature DC (imDC) under low dose of GM-CSF (2 ng/mL) and mature DC (mDC) under high dose of GM-CSF/IL-4 (20 ng/mL) has been described previously (92). DC at day 6 in culture were further pulsed with OVA protein (0.1 mg/mL) in AIM-V medium (GIBCO) for overnight culture and termed DC_{OVA}. DC derived from H-2K^{b} KO mice were termed DC (K^{b-/-}).

Generation and Purification of Exosomes

Exosomes (EXO) were isolated as described previously (64,65). Briefly, culture supernatants of mDC_{OVA} were subjected to four successive centrifugations at 300×g for 5 min to remove cells, 1,200×g for 20 min and 10,000×g 30 min to remove cellular debris and 100,000×g for 1 h to pellet exosomes. The EXO pellets were washed twice in a large volume of PBS and recovered by centrifugation at 100,000×g for 1 h. The amount of exosomal proteins recovered was measured using Bradford assay (Bio-Rad, Richmond, Calif.). EXO derived from mDC_{OVA} of wild-type C57BL/6 and C57BL/6.1 mice were termed as EXO_{OVA} and EXO_{6.1}, respectively. EXO derived from mDC_{OVA} of H-2K^{b}, CD54, CD80 KO mice were termed (K^{b-/-})EXO, (CD54^{-/-})EXO and (CD80^{-/-})EXO, respectively. To obtain CFSE-labeled EXO_{CFSE}, mDC were stained with 0.5 μM CFSE at 37° C. for 20 minutes and washed three times with PBS (93,94), and then pulsed with OVA protein in AIM-V serum-free medium for overnight culture. The CFSE-labeled EXO_{CFSE} were then harvested and purified from the culture supernatants as described above.

Phenotypic Characterization of DC and Exosomes

For phenotypic analysis of DC, both imDC_{OVA} and mDC_{OVA} were stained with a panel of biotin-labeled and FITC-labeled Abs and analyzed by flow cytometry. For phenotypic analysis of EXO, EXO_{OVA} (25-40 μg) were incubated with a panel of FITC-conjugated Abs on ice for 30 min, and then analyzed by flow cytometry as previously described (95). To determine the optimal voltage suitable for EXO analysis, Dynal M450 beads with a size of 4.5 μm in diameter (DYNAL Inc, Lake Success, N.Y.) were used as a size control by flow cytometric analysis (95) using FACScan (Coulter EPICS XL, Beckman Coulter, San Diego, Calif.). For analysis of expression of intracellular molecules such as TLR9 and MyD88, DC and exosomes were permeablized using Cytofix/Cytoperm Plus Kit (Pharmingen Inc) according to company's protocol before Ab staining. Isotype-matched biotin-labeled or FITC-conjugated Abs were used as controls.

Preparation of T Cells

Naïve OVA-specific T cells were isolated from OVA-specific TCR transgenic OT I and OT II mouse spleens, respectively, and enriched by passage through nylon wool columns. OT II CD4⁺ and OT I CD8⁺ T cells were then purified by negative selection using anti-mouse CD8 (Ly2) or CD4 (L3T4) paramagnetic beads (DYNAL Inc) (63) to yield populations that were >98% CD4⁺/Vα2Vβ5⁺ or CD8⁺/Vα2Vβ5⁺, respectively.

Exosome Uptaken by DC

Both mDC and imDC were co-cultured with EXO_{OVA} (10 μg/1×10⁶ DC) in 0.5-1 mL AIM-V medium at 37° C. for 6 hrs, washed twice with PBS and termed mDC_{EXO} and imDC_{EXO}. To assess EXO absorption, mDC and imDC were co-cultured with EXO_{CFSE} or EXO_{6.1} (10 μg/1×10⁶ DC) and then analyzed for CFSE staining and expression of CD45.1 molecule, respectively, by flow cytometry. To investigate the molecular mechanisms involved in EXO absorption, mDC(K^{b-/-}) were incubated with a panel of Abs specific for H-2K^{b}, Ia^{b}, LFA-1, DEC205 and DC-SIGN (15 μg/mL), the fusion protein CTLA-4/IgG (10 μg/mL), an inhibitor of actin polymerization CCD (15 μg/mL), D-mannose, D-glucose, D-fucose and D-glucosamine (5 mM), and EDTA (50 mM), respectively, on ice for 30 min before and during co-culturing with EXO_{OVA}.

In Vitro T Cell Proliferation Assay

To assess the functional effect of DC-derived EXO, an in vitro CD8⁺ T cell proliferation assay was then performed. EXO_{OVA} (10 μg/ml) and their 2-fold dilutions were cultured with a constant number of naïve OT I CD8⁺ T cells (1×10⁵ cells/well). To test whether pMHC I complexes of EXO_{OVA} uptaken by DC are functional, mDC (0.3×10⁵ cells/well) and imDC (0.3×10⁵ cells/well) were co-cultured with EXO_{OVA}, and their 2-fold dilutions for 4 hrs, and then a constant number of naïve OT I CD8⁺ T cells (1×10⁵ cells/well) were added into each well. To examine the molecular mechanism, before OT I CD8⁺ T cells were added, a panel of reagents including anti-H-2 Kb and LFA-1 Abs, and CTLA-4/Ig fusion protein (each 10 μg/ml), a mixture of the above reagents (as mixed reagents) and a mixture of isotype-matched irrelevant Abs (as control reagents) were added to the culture of mDC and EXO_{OVA}, respectively. After culturing for 48 hrs, thymidine incorporation was determined by liquid scintillation counting (34).

Tetramer Staining and ELISPOT Assays

C57BL/6 or CD4 KO mice were i.v. immunized with EXO_{OVA} (10 μg/mouse) and irradiated (4,000 rad) DC_{OVA}, mDC_{EXO} and imDC_{EXO} (0.5×10⁶ cells/mouse), respectively. In one set of experiment, the blood samples were incubated with ten microliters of PE-conjugated H-2K^{b}/OVA₂₅₇₋₂₆₄ tetramer (Beckman Coulter, Mississauga, Ontario, Canada) and FITC-conjugated anti-CD8 (PK135) for 30 min at room temperature. The erythrocytes were then lysed using lysis/fixed buffer (Beckman Coulter). The cells were analyzed by flow cytometry. In another set of experiments, the above immunized mice were i.v. boosted with irradiated DC_{OVA} (0.5×10⁶) three months after immunization, the blood samples were analyzed by flow cytometry 4 days after the boost. In ELISPOT assay (96), splenocytes (1×10⁶ cells) harvested from mice 6 days after the primary immunization were seeded into each well of filtration plates (96 wells; Millipore, Bedford, Mass.) in absence (as control) or presence of OVA 1 (2 μM), which were previously coated with purified anti-IFN-γ Ab for 24 h and blocked with 10% FCS. The plates were then incubated at 37° C. for 24 hr. After washing, biotin-conjugated anti-IFN-γ mAb were added and incubated for 2 hr at room temperature. The plates were then washed 3 times with distilled water. The streptavidin-alkaline phosphatase (Invitrogen, Carlsbad, Calif.) was added, and the plates were incubated for 1-2 hr at room temperature. After 3 washes with distilled water, the alkaline phosphatase substrate BCIP/NBT (Sigama) was added, and the color was developed according to the manufacturer's instructions. Spots were counted under a microscope.

Animal Studies

To examine protective antitumor immunity, wild-type C57BL/6, CD4 KO or CD8 KO mice (n=8) were injected i.v. with EXO_{OVA} (10 μg/mouse), and irradiated (4,000 rad) DC_{OVA} (0.05-0.5×10⁶ cells/mouse), mDC_{EXO} (0.05-0.5×10⁶ cells/mouse) and imDC_{EXO} (0.5×10⁶ cells/mouse), respectively. The immunized mice were i.v. challenged with 0.5×10⁶ BL6-10_{OVA} 6 days or 3 months after immunization. To examine the therapeutic effect on established tumors, wild-type C57BL/6 mice (n=15) were firstly injected i.v. with 0.5×10⁶ BL6-10_{OVA} tumor cells. After 5 days, mice were immunized with irradiated DC_{OVA} and mDC_{EXO} (1.0×10⁶ cells/mouse). The mice were sacrificed 4 weeks after tumor cell injection and the lung metastatic tumor colonies were counted in a blind fashion. Metastases on freshly isolated lungs appeared as discrete black pigmented foci that were easily distinguishable from normal lung tissues and confirmed by histological examination. Metastatic foci too numerous to count were assigned an arbitrary value of >100 (63).

Results

Phenotypical Characterization of DC and EXO

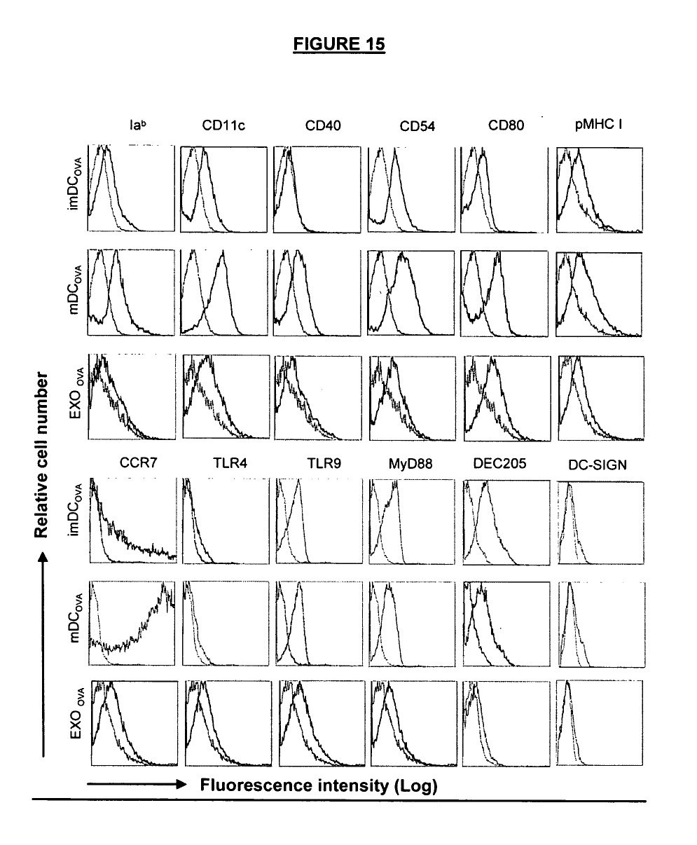

Immature DC (imDC) displayed low expression of MHC Class II (Ia^{b}), co-stimulatory molecule CD80 and chemokine receptor CCR7 and were deficient in CD40 expression (FIG. 15), each of which plays a critical role in T cell activation. Mature DC (mDC) exhibited higher expression of the above molecules compared with the imDC (FIG. 15). Both imDC and mDC displayed expression of CD11c, adhesion molecule CD54, Toll-like receptors TLR4 and TLR9, MyD88, C-type lectins DEC205 with ligand specificity for mannose and DC-SIGN with ligand specificity for mannan, Le^{X}, etc. They expressed similar amount of PMHC I after pulsing with OVA protein. The expression of pMHC 1, MHC class II (Ia^{b}), CD11c, CD40, CD54, CD80, CCR7, TLR4, TLR9, MyD88, DEC205 and DC-SIGN were also detected on EXO_{OVA}, but at a lower level than mDC_{OVA} (FIG. 15).

DC Uptake Exosomal Molecules

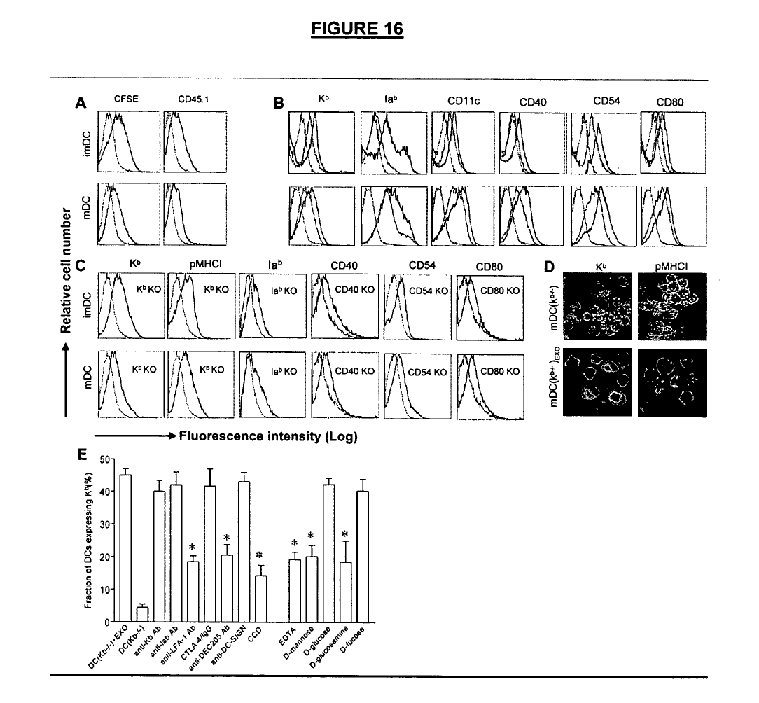

To assess EXO uptaken by DC, mDC and imDC were incubated with CFSE-labeled EXO_{CFSE} and then analyzed by flow cytometry. As shown in FIG. 16A, the CFSE dye was detectable on both mDC and imDC, indicating that DC can absorb EXO. To further confirm it, both mDCs and imDCs were also incubated with EXO_{6.1} expressing CD45.1 molecule. As shown in FIG. 16A, both mDCs and imDCs acquired CD45.1 after incubation with EXO_{6.1}. Furthermore, other EXO molecules such as MHC class I and II, CD11c, CD40, CD54 and CD80 molecules can also be transferred onto both imDC and mDC (FIG. 16B). To confirm the acquisition, EXO with DC derived from C57BL/6 mice were incubated with different gene knockout (KO). As shown in FIG. 16C, the original mDC and imDC derived from gene KO mice did not express H-2K^{b}, pMHC I, Ia^{b}, CD40, CD54 and CD80, respectively. However, each of them was displayed on DC after incubation with EXO_{OVA}, indicating that an increased expression of the above molecules is due to acquisition of EXO molecules by DC. The transfer of exosomal pMHC I onto DC, which is critical in stimulation of OVA-specific CTL responses, was also confirmed by fluorescence microscopy (FIG. 16D),

EXO Uptaken by DC is Mediated by LFA-1/CD54 and C-Type Lectin/C-Type Lectin Receptor Interactions

To elucidate the molecular mechanisms involved in EXO uptake, an inhibition assay was performed using a panel of blocking reagents. As shown in FIG. 16E, EXO uptake by DC was significantly decreased by blocking with the anti-LAF-1 and anti-DEC205 Abs (p<0.05), but not with the anti-H-2K^{b}, anti-Ia^{b} and anti-DC-SIGN Abs, and the CTLA-4/Ig fusion protein, indicating that LFA-1/CD54 and C-type lectin/mannose-rich CLR interactions are involved in EXO uptake. In addition, EXO uptaken by DC was also significantly reduced (P<0.05) after treatment of CCD (an inhibitor of actin polymerization), indicating that the actin cytoskeleton is crucial for EXO uptake. Since the interaction of C-type lectin and CLR is calcium-dependent (97), EDTA capable of chelating calcium ions was then used. As shown in FIG. 16E, EDTA (50 mM) significantly reduced EXO uptake by DC (P<0.05), confirming that EXO uptake by DC is mediated with C-type lectin/CLR interactions. To further confirm the involvement of C-type lectin/mannose-rich CLR interaction in EXO uptake, a panel of monosaccharides in the blocking test was used. Interestingly, both D-mannose and D-glucosamine, but not D-glucose and D-fucose significantly reduced EXO uptake (P<0.05), indicating that EXO uptaken by DC is mediated by interaction between C-type lectin and mannose/glucosamine-rich CLR.

EXO-Targeted DC Stimulate Naïve CD8⁺ T Cell Proliferation In Vitro

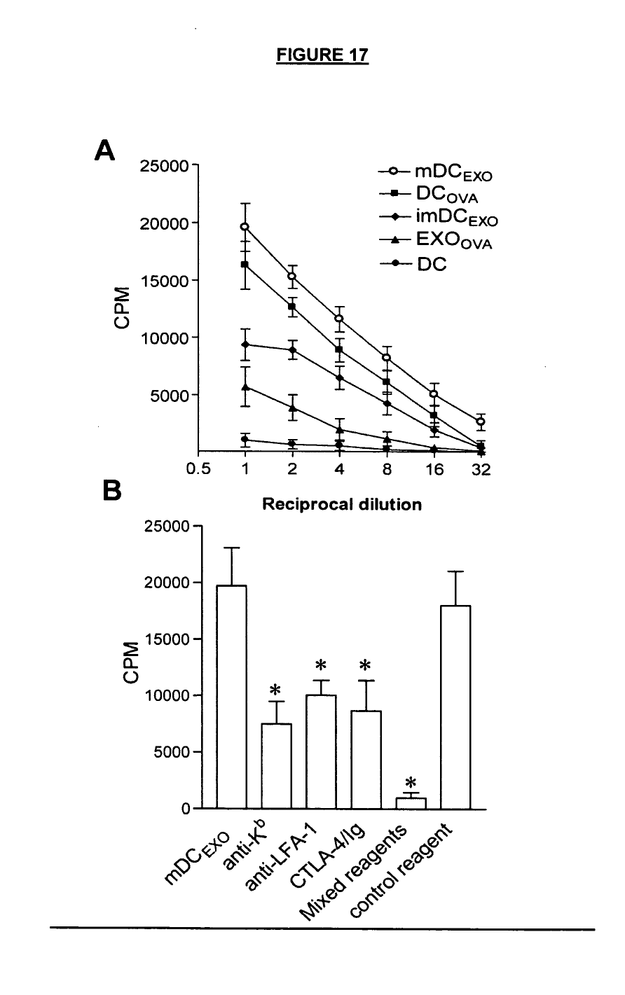

Since EXO harbor immune molecules, they have potent effect in stimulation of CD8⁺ T cells (87). The inventor's data showed that EXO_{OVA} stimulated OT I CD8⁺ T cell proliferation in vitro, but in much less efficiency than DC_{OVA}, mDC_{EXO} and imDC_{EXO}, indicating that EXO require DC to more efficiently activate naïve CD8⁺ T cells (FIG. 17A). Among them, EXO-uptaken (targeted) mDC_{EXO} is the most efficient stimulator. To investigate the molecular mechanism involved in CD8⁺ T cell proliferation, a panel of reagents was added to the cell cultures. As shown in FIG. 17B, the anti-MHC class I, anti-LFA-1 Ab, and CTLA-4/1 g could significantly inhibit the OT I CD8⁺ T cell proliferative response in the co-cultures by 62%, 49% and 56% (p<0.05), respectively. A more effective inhibition in proliferation of CD8⁺ T cell by 95% were observed in the mixed reagents group (p<0.05), indicating that the CD8⁺ T cell proliferation is critically dependent on pMHC I/TCR specificity, and greatly affected by costimulations (CD80/CD28 and CD54/LFA-1).

EXO-Targeted DC Activate CD8⁺ T Cell Proliferation In Vivo

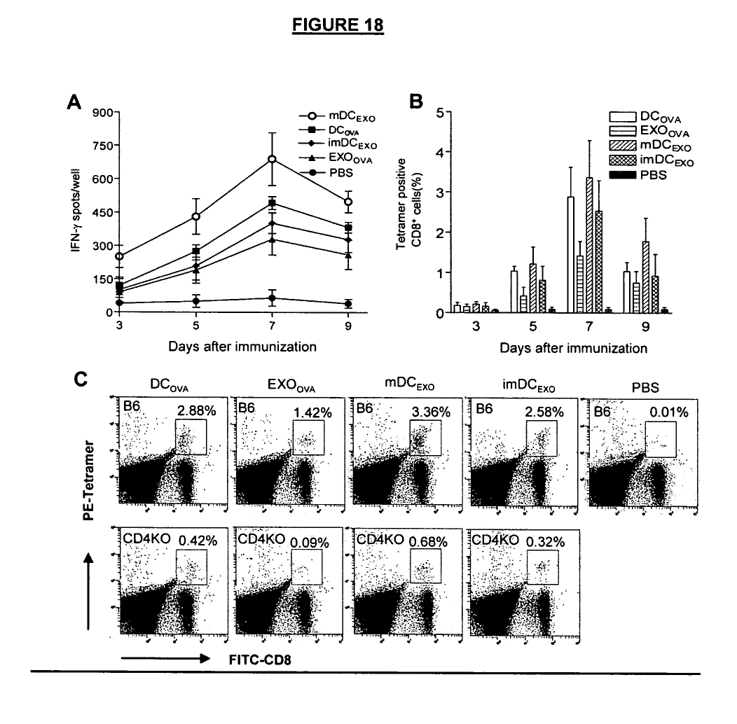

To assess whether EXO-targeted DC can also stimulate CD8⁺ T cell proliferation in vivo, kinetic studies using ELISPOT and tetramer staining assays were performed (47). As shown in FIGS. 18A and 18B, the OVA-specific and IFN-γ-secreting CD8⁺ T cell proliferative responses peaked at day 7 and then declined at day 9 after immunization with DC_{OVA}, EXO_{OVA}, mDC_{EXO} and imDC_{EXO}, respectively. EXO_{OVA} itself could only induce an average of 319 IFN-γ-secreting cells/10⁶ splenocytes or 1.42% tetramer-positive CD8⁺ T cells of the total white blood cells at day 7 after immunization, indicating that EXO_{OVA} can induce activation of naïve Ag-specific CD8⁺ T cell responses in vivo, but in a much less extent compared with DC_{OVA} (504 IFN-γ-secreting cells/10⁶ splenocytes and 2.88% tetramer-positive CD8⁺ T cells). Interestingly, mDC_{EXO} induced the strongest CD8⁺ T cell responses (680 IFN-γ-secreting cells/10⁶ splenocytes and 3.36% tetramer-positive CD8⁺ T cells), indicating that EXO-targeted mDC_{EXO} can efficiently prime naïve CD8⁺ T cell responses in vivo. The inventor's data also showed that both DC_{OVA}, mDC_{EXO} and imDC_{EXO}, but not EXO_{OVA}, can still stimulate OVA-specific CD8⁺ T cell proliferation (0.42%, 0.68% and 0.32% tetramer-positive CD8⁺ T cells of the total white blood cells) (FIG. 18C), indicating that EXO_{OVA} mainly induce CD4⁺ Th-dependent CD8⁺ CTL responses, whereas DC_{OVA}, mDC_{EXO} and imDC_{EXO} mainly induce CD4⁺ Th-independent, but also induce some CD4⁺ Th-dependent CD8⁺ CTL responses.

EXO-Targeted DC Stimulate CD8⁺ T Cell Differentiation into CTL Effectors In Vitro and In Vivo

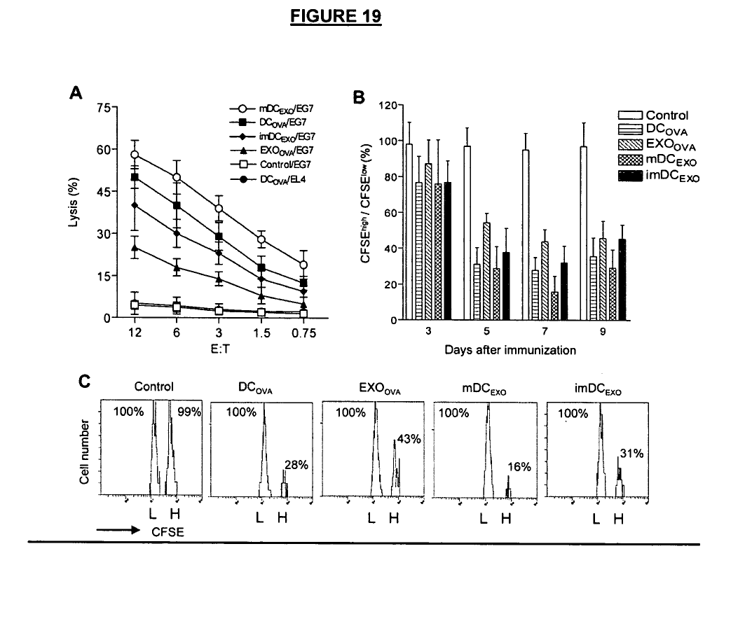

In in vitro cytotoxicity assay, CD8⁺ T cells activated by EXO_{OVA} in vitro displayed killing activities against EG7 cells (25% killing; E:T ratio, 12:1), but much weaker than those activated by DC_{OVA}, mDC_{EXO} and imDC_{EXO} (50%, 58% and 39%; E: T ratio, 12:1) (FIG. 19A), respectively. No killing activities against its parental EL4 tumor cells were detectable, indicating that the killing activity of these CTLs is OVA specific. In in vivo cytotoxicity assay, OVAI peptide-pulsed splenocytes that had been strongly labeled with CFSE (CFSE^{high}) as well as the control Mut1 peptide-pulsed splenocytes that had been weakly labeled with CFSE (CFSE^{low}) were adoptively transferred into the recipient mice that had been vaccinated with EXO_{OVA}, DC_{OVA}, mDC_{EXO} and imDC_{EXO}, respectively. The peak of loss of CFSE^{high} target cells occurred at day 7 after immunization in all tested groups (FIG. 19B). No CFSE^{high} target cells loss (>2%) were observed in mice immunized with PBS. As expected, there was substantial loss of the CFSE^{high} cells in the immunized mice. Among them, the mice immunized with mDC_{EXO} and EXO_{OVA} had the largest (84%) and the least (57%) losses of the CFSE^{high} target cells, respectively (FIG. 19C), indicating that EXO-targeted mDC_{EXO} can most efficiently stimulate CD8⁺ T cells differentiating into CTL effectors.

EXO-Targeted DC Induce Stronger Immunity Against Lung Tumor Metastases

As shown in Exp I of Table 3, all the mice injected with PBS had large numbers (>100) of lung metastatic tumor colonies. EXO_{OVA} vaccine only protected 5/8 (63%) mice as did similarly imDC_{EXO} vaccine, whereas both DC_{OVA} and mDC_{EXO} vaccines induced complete immune protection against BL6-10_{OVA} tumor challenge in 8/8 (100%) immunized mice. The specificity of the protection was confirmed with the observation that mDC_{EXO} did not protect against BL6-10 tumors that did not express OVA, with all mice having large numbers (>100) of lung metastatic tumor colonies after tumor cell challenge. The protective immunity derived from DC_{OVA} and mDC_{EXO} vaccines mostly maintained in CD4 KO mice, but completely lost in CD8 KO mice, confirming that DC_{OVA}- and mDC_{EXO}-derived antitumor immunity is mainly CD4⁺ Th-independent and mediated by CD8⁺ T cells. To compare the efficiency of antitumor immunity, different doses of DC_{OVA} and mDC_{EXO} were administered. As shown in Exp II of Table 3, mDC_{EXO} vaccination at lower doses (0.05-0.2×10⁶ cells per mouse) demonstrated more efficient protection than DC_{OVA}, though both of them at high dose (0.5×10⁶ cells) all showed 100% immune protection against BL6-10_{OVA} tumor, indicating that mDC_{EXO} can induce stronger antitumor immunity than DC_{OVA}.

EXO-Targeted DC Eradicate Established Tumors

To investigate the therapeutic effect of EXO-targeted DC on established tumors, mice were firstly injected with BL6-10_{OVA} tumor cells. After 5 days, the mice were then immunized with DC_{OVA} and mDC_{EXO}. As shown in Exp III of Table 3, 13 out of 15 (87%) mice with mDC_{EXO} immunization were tumor free compared with only 7 out of 15 (47%) mice cured in DC_{OVA} group, indicating that EXO-targeted mDC_{EXO} can more efficiently eradicate established tumors than DC_{OVA}.

EXO-Targeted DC Induce Strong Long-Term OVA-Specific CD8⁺ T Cell Memory

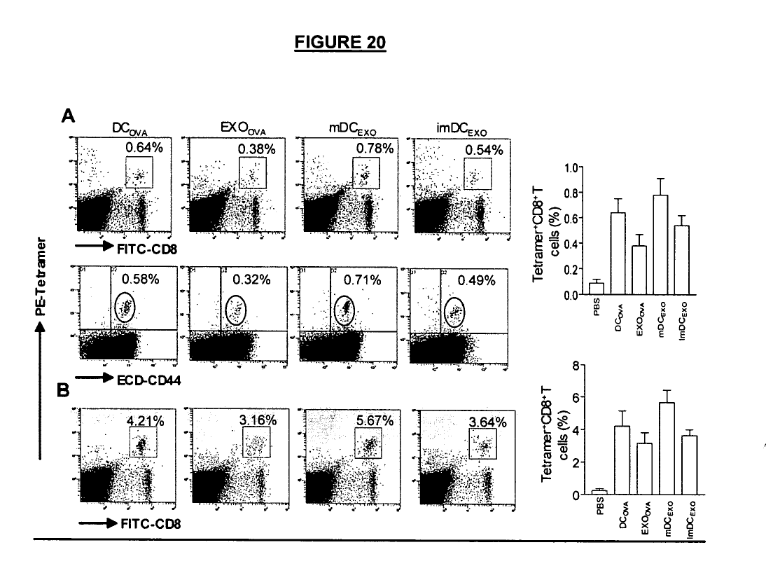

Active CD8⁺ T cells can become long-lived memory T (Tm) cells after adoptive transfer in vivo (75). Since mDC_{EXO} stimulated CD8⁺ T cell differentiation into CTL effectors in vitro and in vivo, these activated CD8⁺ T cells were assessed to determine whether can become long-lived Tm cells. As shown in FIG. 20A, three months after the immunization, 0.64%, 0.38%, 0.78% and 0.54% CD8⁺ T cells expressing H-2K^{b}/OVA₂₅₇₋₂₆₄ tetramer-specific TCR were detected in peripheral blood of mice immunized with DC_{OVA}, EXO_{OVA}, mDC_{EXO} and imDC_{EXO}, respectively. These OVA-specific CD8⁺ T cells were also CD44, a Tm marker (68), indicating that all these vaccines can induce development of OVA-specific CD8⁺ Tm cells. Among them, mDC_{EXO} represent the strongest one. In order to investigate the functionality of these CD8⁺ Tm cells, the immunized mice were boosted with DC_{OVA}. The recall responses were examined using H-2K^{b}/OVA₂₅₇₋₂₆₄ tetramer staining on day 4 after the boost. As shown in FIG. 20B, there was few OVA-specific CD8⁺ T cells detected in peripheral blood of the mice, which were injected with PBS three months ago and boosted with DC_{OVA} four days ago, indicating that the primary proliferation of OVA-specific CD8⁺ T cells is almost undetectable by DC_{OVA} boost at that time point. As expected, the number of CD8⁺ T cells expressing H-2K^{b}/OVA₂₅₇₋₂₆₄ tetramer-specific TCR was expanded by 6-7 folds in the immunized mice after the boost, indicating that these CD8⁺ Tm cells are functional. In another set of experiments, the above immunized mice were challenged with BL6-10_{OVA} tumor cells 3 months after the immunization. As expected, the control mice died of lung metastasis. In contrast, mice immunized with mDC_{EXO}, imDC_{EXO} and DC_{OVA} were tumor free (Exp. IV of Table 3), confirming that these CD8⁺ Tm cells remained functional.

Discussion

In recent years, EXO research has been stimulated by the finding that APC such as B lymphocytes and DC secrete EXO during exocytic fusion of multivesicular MHC class II compartments with the cell surface (64,65). Formation of EXO occurs in MHC class II enriched compartments (MIIC) by macroautophagy of the internal membrane, then EXO are exocytosed by direct fusion of MIIC with plasma membrane. EXO from BM-DCs display immunologically important molecules such as MHC class I and II, CD54 and co-stimulatory molecule CD86 (98,99,95) necessary for induction of immune responses. EXO-based vaccines have been shown to induce antitumor immunity (24-28). However, its efficiency was less effective because it only induced either prophylactic immunity in animal models (24-28) or very limited immune responses in clinical trials (86). In addition, the mechanism of EXO-mediated immunity in vivo is still poorly understood. The potential pathway of EXO-mediated immunity may be through uptake of EXO by the host imDC.

In this study, DC_{OVA}-derived EXO were systemically characterized by flow cytometry. The inventor demonstrated that, in addition to the previously reported MHC class I and II, CD11b, CD54 and CD86 molecules (98,99,95), EXO also expressed CD11c, co-stimulatory molecule CD80, chemokine receptor CCR7, mannose-rich C-type lectin receptor DEC205 and Toll-like receptors TLR4 and TLR9. In addition, for the first time, the inventor also demonstrated that EXO also expressed MHC class I/OVA I peptide (PMHC I) complexes and contained intracellular molecules such as MyD88 related to signal transduction, indicating that EXO carry all the immunologically important molecules as DC for induction of immune responses.

Membrane transfer has been reported in systems requiring or not requiring cell to cell contact (100). Knight et al have shown that DC acquire Ag from cell-free DC supernatants (101). In this study, the inventor demonstrated that EXO can be uptaken by mDC and imDC. The expression of immunologically important molecules such as MHC class II, CD40, CD54 and CD80 was all enhanced on DC after EXO uptake. The non-specific LFA-1/CD54 interaction between EXO and DC was involved in the EXO uptake, which is consistent with a previous report by Sprent et al (87). In immune system, C-type lectins and C-type lectin receptors (CLR) have been shown to act as both the adhesion and the pathogen recognition receptors (102). C-type lectins include mannose receptor (MMR) family such as DEC205 (103) and type II receptors such as DC-SIGN (104). In addition to the adhesion effect, DEC205 and DC-SIGN have been demonstrated to mediate Ag uptake (105,106). DC-SIGN also mediates the contact between DC and T cells by binding to ICAM-3 (104) and the rolling of DC on endothelium by interacting with ICAM-2 (107). Interestingly, the inventor found that the anti-DEC205, but not the anti-DC-SIGN antibody can significantly reduce EXO uptake by DC, indicating that the interaction of C-type lectin and mannose-rich CLR may be involved in EXO uptake by DC. A panel of monosaccharides in the blocking test was then used. Interestingly, both D-mannose and D-glucosamine significantly reduced EXO uptake. Therefore, for the first time, the inventor elucidated another important molecular mechanism of EXO uptake by DC (i.e. C-type lectin/mannose[glucosamine]-rich CLR interaction).

EXO_{OVA} derived from OVA protein-pulsed DC_{OVA} can stimulate OT I CD8⁺ T cell proliferation in vitro, which is also consistent with a previous report by Sprent et al (87), but in a relatively mild fashion. In comparison, mature DC with EXO uptake (mDC_{EXO}) can more strongly stimulate CD8⁺ T cell proliferation and differentiation into effector CTL than immature DC with EXO uptake (imDC_{EXO}), tumor Ag-pulsed mature DC (DC_{OVA}) and EXO_{OVA}. It is because mDC_{EXO} express higher level of MHC class II, CD40, CD54 and CD80 than imDC_{EXO} and OVA-pulsed DC_{OVA}. It is also because EXO vaccine needs DC adjuvant through EXO uptake by the host immature DC for induction of immune responses (26,108), and may thus be equivalent to imDC_{EXO} vaccine. In addition, EXO-targeted mDC_{EXO} vaccine can further induce more effective OVA-specific CTL responses against OVA-expressing EG7 tumor cells and antitumor immunity as demonstrated in our lung metastasis animal model. Since tumor cell-derived EXO is a good source of tumor antigens, EXO-targeted-DC vaccine may become a feasible one in combating tumors by using EXO purified from cancer patient's ascites, which are then uptaken by in vitro-activated DC derived from patient's peripheral blood monocytes. Thus, EXO-targeted DC vaccine may represent a novel and feasible EXO- and DC-based vaccine approach against tumors.

Taken together, the inventor's data showed that OVA protein-pulsed DC_{OVA}-derived exosomes (EXO_{OVA}) can be uptaken by DC via LFA-1/CD54 and C-type lectin/mannose(glucosamine)-rich CLR interactions. EXO-targeted mDC_{OVA} expressing higher level of PMHC I and costimulatory CD40, CD54 and CD80 molecules can more efficiently stimulate naïve OVA-specific CD8⁺ T cell proliferation in vitro and in vivo, and induce OVA-specific CTL responses, antitumor immunity and CD8⁺ T cell memory in vivo than EXO_{OVA} and DC_{OVA}. Therefore, the EXO-targeted mDC_{OVA} may represent a new highly effective DC-based vaccine in induction of antitumor immunity.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Vaccination with CD4⁺ Th-APC protects against lung tumor
metastases in mice

| | | | |
|---|---|---|---|
| | Tumor cell | Tumor-bearing | Median number of |
| Immunization | challenge | mice (%) | lung tumor colonies |

Experiment I^{a}

| | | | |
|---|---|---|---|
| DC_{OVA} | BL6-10_{OVA} | 0/8 (0) | 0 |
| Th-APCs | BL6-10_{OVA} | 0/8 (0) | 0 |
| Con A-OT II cells | BL6-10_{OVA} | 8/8 (100) | >100 |
| PBS | BL6-10_{OVA} | 8/8 (100) | >100 |
| Th-APCs | BL6-10 | 8/8 (100) | >100 |
| PBS | BL6-10 | 8/8 (100) | >100 |

Experiment II^{b}

| | | | |
|---|---|---|---|
| Th-APCs (B6 mice) | BL6-10_{OVA} | 0/8 (0) | 0 |
| Th-APCs (CD4 KO) | BL6-10_{OVA} | 0/8 (0) | 0 |
| Th-APCs (CD8 KO) | BL6-10_{OVA} | 8/8 (100) | >100 |

^{a}In experiment I, C57BL/6 mice (n = 8) were immunized with DC_{OVA}, Th-APCs, Con A-OT II cells or PBS. Following the immunization, each mouse was challenged i.v. with OVA transgene-expressing (BL6-10_{OVA}) or wild-type BL6-10 tumor cells. The mice were sacrificed 4 weeks after tumor cell challenge and the numbers of lung metastatic tumor colonies were counted. One representative experiment of two is shown.
^{b}In experiment II, wild-type C57BL/6 (B6) and CD4 or CD8 KO mice (n = 8) were immunized with Th-APCs. Following the immunization, each mouse was challenged i.v. with OVA transgene-expressing (BL6-10_{OVA}) tumor cells. The mice were sacrificed 4 weeks after tumor cell challenge and the numbers of lung metastatic tumor colonies were counted. One representative experiment of two is shown.

a. In experiment 1, C57BL/6 mice (n=8) were immunized with DC_{OVA}, Th-APCs, Con A-OT II cells or PBS. Following the immunization, each mouse was challenged i.v. with OVA transgene-expressing (BL6-10_{OVA}) or wild-type BL6-10 tumor cells. The mice were sacrificed 4 weeks after tumor cell challenge and the numbers of lung metastatic tumor colonies were counted. One representative experiment of two is shown.

b. In experiment II, wild-type C57BL/6 (B6) and CD4 or CD8 KO mice (n=8) were immunized with Th-APCs. Following the immunization, each mouse was challenged i.v. with OVA transgene-expressing (BL6-100vA) tumor cells. The mice were sacrificed 4 weeks after tumor cell challenge and the numbers of lung metastatic tumor colonies were counted. One representative experiment of two is shown.

TABLE 2
Exosome-targeted CD4⁺ T cell vaccine protects against lung tumor
metastases

| | | | |
|---|---|---|---|
| | | | Median number |
| | Tumor cell | Tumor growth | of lung |
| Vaccines^{A} | challenge | incidence (%) | tumor colonies |

Exp. I.

| | | | |
|---|---|---|---|
| DC_{OVA} | BL6-10_{OVA} | 0/8 (0) | 0 |
| nT_{EXO} | BL6-10_{OVA} | 2/8 (25) | 27 ± 16 |
| aT_{EXO} | BL6-10_{OVA} | 0/8 (0) | 0 |
| PBS | BL6-10_{OVA} | 8/8 (100) | >100 |
| nT_{EXO} | BL6-10 | 8/8 (100) | >100 |
| aT_{EXO} | BL6-10 | 8/8 (100) | >100 |
| PBS | BL6-10 | 8/8 (100) | >100 |

Exp. II.

| | | | |
|---|---|---|---|
| aT_{EXO} (B6) | BL6-10_{OVA} | 0/8 (0) | 0 |
| aT_{EXO} (CD4KO) | BL6-10_{OVA} | 2/8 (25) | 14 ± 13 |
| aT_{EXO} (CD8KO) | BL6-10_{OVA} | 8/8 (100) | >100 |

Exp. III

| | | | |
|---|---|---|---|
| DC_{OVA} | BL6-10_{OVA} | 0/8 (0) | 0 |
| aT_{EXO} | BL6-10_{OVA} | 0/8 (0) | 0 |
| PBS | BL6-10_{OVA} | 8/8 (100) | >100 |

A. In experiment 1, C57BL/6 mice (n=8) were immunized with DC_{OVA}, nT_{EXO} and aT_{EXO} cells or PBS. In experiment II, wild-type C57BL/6 (B6) and CD4 or CD8 KO mice (n=8) were immunized with aT_{EXO} cells. Six days after the immunization, each mouse was challenged i.v. with OVA transgene-expressing (BL6-10_{OVA}) or wild-type BL6-10 tumor cells. In experiment III, C57BL/6 mice (n=8) were immunized with DC_{OVA}, aT_{EXO} cells or PBS. Three months after the immunization, each mouse was challenged i.v. with BL6-10_{OVA} tumor cells. The mice were sacrificed 4 weeks after tumor cell challenge and the numbers of lung metastatic tumor colonies were counted. One representative experiment of three is shown.

TABLE 3
Exosome-targeted DC vaccine protects against lung tumor
metastases

| | | | |
|---|---|---|---|
| | | | Median number |
| | Tumor cell | Tumor growth | of lung |
| Vaccines | challenge | incidence (%) | tumor colonies |

Exp. I.

| | | | |
|---|---|---|---|
| DC_{OVA} | BL6-10_{OVA} | 0/8 (0) | 0 |
| EXO_{OVA} | BL6-10_{OVA} | 3/8 (37) | 27 ± 6 |
| mDC_{EXO} | BL6-10_{OVA} | 0/8 (0) | 0 |
| imDC_{EXO} | BL6-10_{OVA} | 2/8 (25) | 16 ± 5 |
| PBS | BL6-10_{OVA} | 8/8 (100) | >100 |
| DC_{OVA} | BL6-10 | 8/8 (100) | >100 |
| mDC_{EXO} | BL6-10 | 8/8 (100) | >100 |
| DC_{OVA} (CD4KO) | BL6-10_{OVA} | 2/8 (25) | 15 ± 7 |
| mDC_{EXO} (CD4KO) | BL6-10_{OVA} | 1/8 (12) | 13 |
| DC_{OVA} (CD8KO) | BL6-10_{OVA} | 8/8 (100) | >100 |
| mDC_{EXO} (CD8KO) | BL6-10_{OVA} | 8/8 (100) | >100 |

Exp. II.

| | | | |
|---|---|---|---|
| 0.5 × 10⁶ DC_{OVA} | BL6-10_{OVA} | 0/8 (0) | 0 |
| 0.2 × 10⁶ DC_{OVA} | BL6-10_{OVA} | 2/8 (25) | 15 ± 6 |
| 0.1 × 10⁶ DC_{OVA} | BL6-10_{OVA} | 4/8 (50) | 28 ± 9 |
| 0.05 × 10⁶ DC_{OVA} | BL6-10_{OVA} | 8/8 (100) | 55 ± 14 |
| 0.5 × 10⁶ mDC_{EXO} | BL6-10_{OVA} | 0/8 (0) | 0 |
| 0.2 × 10⁶ mDC_{EXO} | BL6-10_{OVA} | 0/8 (0) | 0 |
| 0.1 × 10⁶ mDC_{EXO} | BL6-10_{OVA} | 1/8 (12) | 16 |
| 0.05 × 10⁶ mDC_{EXO} | BL6-10_{OVA} | 3/8 (37) | 17 ± 8 |
| PBS | BL6-10_{OVA} | 8/8 (100) | >100 |

Exp. III.

| | | | |
|---|---|---|---|
| DC_{OVA} | BL6-10_{OVA} | 8/15 (53) | 35 ± 10 |
| mDC_{EXO} | BL6-10_{OVA} | 2/15 (13) | 9 ± 7 |
| PBS | BL6-10_{OVA} | 15/15 (100) | >100 |

Exp. IV.

| | | | |
|---|---|---|---|
| DC_{OVA} | BL6-10_{OVA} | 0/8 (0) | 0 |
| mDC_{EXO} | BL6-10_{OVA} | 0/8 (0) | 0 |
| imDC_{EXO} | BL6-10_{OVA} | 0/8 (0) | 0 |
| PBS | BL6-10_{OVA} | 8/8 (100) | >100 |

In experiment 1, wild-type C57BL/6, CD4 and CD8 KO mice (n=8) were i.v. immunized with DC_{OVA}, EXO_{OVA}, mDC_{EXO}, imDC_{EXO} or PBS. Six days after immunization, each mouse was challenged i.v. with OVA transgene-expressing BL6-10_{OVA} or wild-type BL6-10 tumor cells. In experiment II. wild-type C57BL/6 mice (n=8) were i.v. immunized with different doses of DC_{OVA} and mDC_{EXO} (0.5-0.05×10⁶ cells/mouse). Six days after immunization, each mouse was challenged i.v. with BL6-10_{OVA} tumor cells.

In experiment III, wild-type C57BL/6 mice (n=15) were first injection i.v. with BL6-10_{OVA} tumor cells. Five days after tumor injection, mice were then immunized i.v. with DC_{OVA} and EXO_{OVA}, respectively.

In experiment IV, wild-type C57BL/6 mice (n=8) were i.v. immunized with DC_{OVA}, EXO_{OVA}, mDC_{EXO}, imDC_{EXO} or PBS. Three months after immunization, each mouse was challenged i.v. with BL6-10_{OVA} tumor cells. The mice were sacrificed 4 weeks after tumor cell challenge and the numbers of lung metastatic tumor colonies were counted. One representative experiment of three is shown.

### FULL CITATIONS FOR REFERENCES REFERRED TO IN THE SPECIFICATION

1. Pardoll, D. 1998. Cancer vaccines. Nat. Med. 4: 525.
2. Keene, J. A., and J. Forman. 1982. Helper activity is required for the in vivo generation of cytotoxic T lymphocytes. J. Exp. Med. 155:768.
3. Mitchison, N. A., and C. O'Malley. 1987. Three-cell-type clusters of T cells with antigen-presenting cells best explain the epitope linkage and noncognate requirements of the in vivo cytolytic response. Eur. J. Immunol. 17:1579.
4. Bennett, S. R., F. R. Carbone, F. Karamalis, J. F. Miller, W. R. Heath. 1997. Induction of a CD8⁺ cytotoxic T lymphocyte response by cross-priming requires cognate CD4⁺ T cell help. J. Exp. Med. 186:65.
5. Ridge, J. P., F. Di Rosa, P. Matzinger. 1998. A conditioned dendritic cell can be a temporal bridge between a CD4⁺ T-helper and a T-killer cell. Nature. 393:474.
6. Bousso, P., and E. Robey. 2003. Dynamics of CD8⁺ T cell priming by dendritic cells in intact lymph nodes. Nat. Immunol. 4:579.
7. Bennett, S. R., F. R. Carbone, F. Karamalis, R. A. Flavell, J. F. Miller, W. R. Heath. 1998. Help for cytotoxic-T-cell responses is mediated by CD40 signalling. Nature. 393:478.
8. Schoenberger, S. P., R. E. Toes, E. I. van der Voort, R. Offringa, C. J. Melief. 1998. T-cell help for cytotoxic T lymphocytes is mediated by CD40-CD40L interactions. Nature. 393:480.
9. Yang, Y., and J. M. Wilson. 1996. CD40 ligand-dependent T cell activation: requirement of B7-CD28 signaling through CD40. Science. 273:1862.
10. Bretscher, P. A. 1999. A two-step, two-signal model for the primary activation of precursor helper T cells. Proc. Natl. Acad. Sci. USA 96:185.
11. Ingulli, E., A. Mondino, A. Khoruts, and M. K. Jenkins. 1997. In vivo detection of dendritic cell antigen presentation to CD4⁺ T cells. J. Exp. Med. 185:2133.
12. Ruedl, C., P. Koebel, M. Bachmann, M. Hess, and K. Karjalainen. 2000. Anatomical origin of dendritic cells determines their life span in peripheral lymph nodes. J. Immunol. 165:4910.
13. Norbury, C. C., D. Malide, J. S. Gibbs, J. R. Bennink, J. W. Yewdell. 2002. Visualizing priming of virus-specific CD8⁺ T cells by infected dendritic cells in vivo. Nat. Immunol. 3:265.
14. Tite, J. P. 1990. Evidence of a role for TNF-alpha in cytolysis by CD4+, class II MHC-restricted cytotoxic T cells. Immunology. 71:208.
15. Rathmell, J. C., M. P. Cooke, W. Y. Ho, J. Grein, S. E. Townsend, M. M. Davis, C. C. Goodnow. 1995. CD95 (Fas)-dependent elimination of self-reactive B cells upon interaction with CD4⁺ T cells. Nature. 376:181.
16. Lanzavecchia, A. and F. Sallusto. 2000. Dynamics of T lymphocyte responses: intermediates, effectors, and memory cells. Science. 290:92.
17. Lenschow, D. J., T. L. Walunas, J. A. Bluestone. 1996. CD28/B7 system of T cell costimulation. Annu. Rev. Immunol. 14:233.
18. Grakoui, A., S. K. Bromley, C. Sumen, M. M. Davis, A. S. Shaw, P. M. Allen, M. L. Dustin. 1999. The immunological synapse: a molecular machine controlling T cell activation. Science. 285:221.
19. Viola, A., S. Schroeder, Y. Sakakibara, A. Lanzavecchia, 1999. T lymphocyte costimulation mediated by reorganization of membrane microdomains. Science. 283:649.
20. Hwang, I., J. F. Huang, H. Kishimoto, A. Brunmark, P. A. Peterson, M. R. Jackson, C. D. Surh, Z. Cai, J. Sprent. 2000. T cells can use either T cell receptor or CD28 receptors to absorb and internalize cell surface molecules derived from antigen-presenting cells. J. Exp. Med. 191:1137.
21. Huang, J. F., Y. Yang, H. Sepulveda, W. Shi, I. Hwang, P. A. Peterson, M. R. Jackson, J. Sprent, Z. Cai. 1999. TCR-Mediated internalization of peptide-MHC complexes acquired by T cells. Science. 286:952.
22. Altieri, S. L., A. N. Khan, and T. B. Tomasi. 2004. Exosomes from plasmacytoma cells as a tumor vaccine. J Immunother 27:282.
23. Wolfers, J., A. Lozier, G. Raposo, A. Regnault, C. Thery, C. Masurier, C. Flament, S. Pouzieux, F. Faure, T. Tursz, E. Angevin, S. Amigorena, and L. Zitvogel. 2001. Tumor-derived exosomes are a source of shared tumor rejection antigens for CTL cross-priming. Nat Med 7:297.
24. Andre, F., N. E. Schartz, M. Movassagh, C. Flament, P. Pautier, P. Morice, C. Pomel, C. Lhomme, B. Escudier, T. Le Chevalier, T. Tursz, S. Amigorena, G. Raposo, E. Angevin, and L. Zitvogel. 2002. Malignant effusions and immunogenic tumour-derived exosomes. Lancet 360:295.
25. Hsu, D. H., P. Paz, G. Villaflor, A. Rivas, A. Mehta-Damani, E. Angevin, L. Zitvogel, and J. B. Le Pecq. 2003. Exosomes as a tumor vaccine: enhancing potency through direct loading of antigenic peptides. J Immunother 26:440.
26. Andre, F., N. Chaput, N. E. Schartz, C. Flament, N. Aubert, J. Bernard, F. Lemonnier, G. Raposo, B. Escudier, D. H. Hsu, T. Tursz, S. Amigorena, E. Angevin, and L. Zitvogel. 2004. Exosomes as potent cell-free peptide-based vaccine. I. Dendritic cell-derived exosomes transfer functional MHC class I/peptide complexes to dendritic cells. J Immunol 172:2126.
27. Chaput, N., N. E. Schartz, F. Andre, J. Taieb, S. Novault, P. Bonnaventure, N. Aubert, J. Bernard, F. Lemonnier, M. Merad, G. Adema, M. Adams, M. Ferrantini, A. F. Carpentier, B. Escudier, T. Tursz, E. Angevin, and L. Zitvogel. 2004. Exosomes as potent cell-free peptide-based vaccine. II. Exosomes in CpG adjuvants efficiently prime naïve Tcl lymphocytes leading to tumor rejection. J Immunol 172:2137.
28. Cho, J. A., D. J. Yeo, H. Y. Son, H. W. Kim, D. S. Jung, J. K. Ko, J. S. Koh, Y. N. Kim, and C. W. Kim. 2005. Exosomes: a new delivery system for tumor antigens in cancer immunotherapy. Int J Cancer 114:613.
29. Liu, Y., H. Huang, Z. Chen, L. Zong, J. Xiang. 2003. Dendritic cells engineered to express the Flt3L stimulate type 1 immune response, and induce enhanced cytotoxic T and natural killer cell cytotoxicities and antitumor immunity. J. Gene Med. 5:668.
30. Kimura, A. K., and J. H. Xiang. 1986. High levels of Met-72 antigen expression: correlation with metastatic activity of B16 melanoma tumor cell variants. J. Nat. Cancer Inst. 76:1247.
31. Mitchell, D., S. Nair, E. Gilboa. 1998. Dendritic cell/macrophage precursors capture exogenous antigen for MHC class I presentation by dendritic cells. Eur. J. Immunol. 28:1923.
32. Li M., G. M. Davey, R. M. Sutherland, K. Christian, A. M. Lew, C. Hirst, F. R. Carbone, R. H. William. 2001. Cell-associated ovalbumin is cross-presented much more efficiently than soluble ovalbumin in vivo. J. Immunol. 166:6099.
33. Slingluff, C. 1996. Tumor antigens and tumor vaccines: peptides as immunogens. Seminars in Surgical Oncol. 12:446.
34. Zhang, W., Z. Chen, F. Li, H. Kamencic, B. Juurlink, J. R. Gordon. Xiang, J. 2003. Tumour necrosis factor-alpha (TNF-alpha) transgene-expressing dendritic cells (DCs) undergo augmented cellular maturation and induce more robust T-cell activation and anti-tumour immunity than DCs generated in recombinant TNF-alpha. Immunology. 108:177.
35. Nishimura, T., K. Iwakabe, M. Sekimot, Y. Ohmi, T. Yahata, M. Nakui, T. Sato, S. Habu, H. Tashiro, M. Sato, A. Ohta. 1999. Distinct role of antigen-specific T helper type 1 (Th1) and Th2 cells in tumor eradication in vivo. J. Exp. Med. 190:617.
36. Bourgeois, C., B. Rocha. C. Tanchot. 2002. A role for CD40 expression on CD8⁺ T cells in the generation of CD8⁺ T cell memory. Science. 297:2060.
37. Azuma, M., H. Yssel, J. H. Phillips, H. Spits, L. L. Lanier. 1993. Functional expression of B7/BB1 on activated T lymphocytes. J. Exp. Med. 177:845.
38. Sansom, D. M. and N. D. Hall. 1993. B7/BB1, the ligand for CD28, is expressed on repeatedly activated human T cells in vitro. Eur. J. Immunol. 23:295.
39. Tatari-Calderone, Z., R. T. Semnani, T. B. Nutman, J. Schlom, H. Sabzevari. 2002. Acquisition of CD80 by human T cells at early stages of activation: functional involvement of CD80 acquisition in T cell to T cell interaction. J. Immunol. 169:6162.
40. Tsang, J. Y., J. G. Chai, R. Lechler. 2003. Antigen presentation by mouse CD4⁺ T cells involving acquired MHC class II:peptide complexes: another mechanism to limit clonal expansion. Blood. 101:2704.
41. Hudrisier, D. P. Bongrand. 2002. Intercellular transfer of antigen-presenting cell determinants onto T cells: molecular mechanisms and biological significance. The FASEB J. 16:477.
42. Huang, H., F. Li, J. Gordon, J. Xiang. 2002. Synergistic enhancement of antitumor immunity with adoptively transferred tumor-specific CD4 and CD8 T cells and intratumoral lymphotactin transgene expression. Cancer Res. 62:2043.
43. Li, Y., N. Wang, H. Li, K. King, R. Bassi, H. Sun, A. Santiago, A. Hooper, P. Bohlen, D. Hicklin. 2002. Active immunization against the vascular endothelial growth factor receptor flk1 inhibits tumor angiogenesis and metastasis. J. Exp. Med. 195:1575.
44. Curtsinger, J., C. Schmidt, A. Mondino, D. Lins, R. Kedl, M. Jenkins, M. Mescher. 1999. Inflammatory cytokines provide a third signal for activation of naïve CD4 and CD8 T cells. J. Immunol. 162:3256.
45. Rosenberg, A. S., T. Mizuochi, S. O. Sharrow, A. Singer. 1987. Phenotype, specificity, and function of T cell subsets and T cell interactions involved in skin allograft rejection. J. Exp. Med. 165:1296.
46. Rees, M. A., A. S. Rosenberg, T. I. Munitz, A. Singer. 1990. In vivo induction of antigen-specific transplantation tolerance to Qala by exposure to alloantigen in the absence of T-cell help. Proc. Natl. Acad. Sci. USA 87: 2765.
47. Wang, J. C. and A. M. Livingstone. 2003. CD4⁺ T cell help can be essential for primary CD8⁺ T cell responses in vivo. J. Immunol. 171:6339.
48. Behrens, G. M. et al. 2004. Helper requirements for generation of effector CTL to islet β cell antigens. J. Immunol. 172:5420.
49. Janssen, E. M., E. E. Lemmens, T. Wolfe, U. Christen, M. G. von Herrath, S. P. Schoenberger. 2003. CD4⁺ T cells are required for secondary expansion and memory in CD8⁺ T lymphocytes. Nature. 421:852.
50. Lyakh, L. A., G. K. Koski, H. A. Young. S. E. Spence, P. A. Cohen, N. R. Rice. 2002. Adenovirus type 5 vectors induce dendritic cell differentiation in human CD14(+) monocytes cultured under serum-free conditions. Blood. 99:600.
51. Mailliard, R. B., Y. I. Son, R. Redlinger, P. T. Coates, A. Giermasz, P. A. Morel. W. J. Storkus, P. Kalinski. 2003. Dendritic cells mediate NK cell help for Th1 and CTL responses: two-signal requirement for the induction of NK cell helper function. J. Immunol. 171:2366.
52. Wang, B., C. N. Christopher, R. Greenwood, J. R. Bennink, J. W. Yewdell, J. A. Frelinger. 2001. Multiple paths for activation of naïve CD8⁺ T cells: CD4⁺-independent help. J. Immunol. 167:1283.
53. Mintern, J. D., G. M. Davey, G. T. Belz, F. R. Carbone, W. R. Heath. 2002. Precursor frequency affects the helper dependence of cytotoxic T cells. J. Immunol. 168:977.
54. Hou, S., X. Y. Mo, L. Hyland, P. C. Doherty. 1995. Host response to Sendai virus in mice lacking class II major histocompatibility complex glycoproteins. J. Virol. 69:1429.
55. Mauri, D., T. Wyss-Coray, H. Gallati, W. J. Pichler. 1995. Antigen-presenting T cells induce the development of cytotoxic CD4⁺ T. cells. I. Involvement of the CD80-CD28 adhesion molecules. J. Immunol. 155:118.
56. Taams, L. S., W. van Eden, M. H. Wauben. 1999. Antigen presentation by T cells versus professional antigen-presenting cells (APC): differential consequences for T cell activation and subsequent T cell-APC interactions. Eur. J. Immunol. 29:1543.
57. Nisini, R., A. Fattorossi, C. Ferlini, R. D'Amelio. 1996. One cause for the apparent inability of human T cell clones to function as professional superantigen-presenting cells is autoactivation. Eur. J. Immunol. 26:797.
58. Satyaraj, E., S. Rath, V. Bal. 1994. Induction of tolerance in freshly isolated alloreactive CD4⁺ T cells by activated T cell stimulators. Eur. J. Immunol. 24:2457.
59. Mannie, M. D., S. K. Rendall, P. Y. Arnold, J. P. Nardella, G. A. White. 1996. Anergy-associated T cell antigen presentation. A mechanism of infectious tolerance in experimental autoimmune encephalomyelitis. J. Immunol. 157:1062.
60. Patel, D. M., P. Y. Arnold, G. A. White, J. P. Nardella, M. D. Mannie. 1999. Class II MHC/peptide complexes are released from APC and are acquired by T cell responders during specific antigen recognition. J. Immunol. 163:5201.
61. Ossendorp, F., E. Mengede, M. Camps, R. Filius, C. J. Melief. 1998. Specific T helper cell requirement for optimal induction of cytotoxic T lymphocytes against major histocompatibility complex class II negative tumors. J. Exp. Med. 187:693.
62. Porgador, A., Yewdell, J. W., Deng, Y., Bennink, J. R. & Germain, R. N. Localization, quantitation, and in situ detection of specific peptide-MHC class I complexes using a monoclonal antibody. Immunity 6, 715-26 (1997).
63. Xiang, J., Huang, H. & Liu, Y. A new dynamic model of CD8⁺ T effector cell responses via CD4⁺ T helper-antigen-presenting cells. J Immunol 174, 7497-505 (2005).

64. Raposo, G. et al. B lymphocytes secrete antigen-presenting vesicles. J Exp Med 183, 1161-72 (1996).
65. Zitvogel, L. et al. Eradication of established murine tumors using a novel cell-free vaccine: dendritic cell-derived exosomes. Nat Med 4, 594-600 (1998).
66. Hao S, Ou Bai, Jinying Yuan, Suzanne Laferte and Xiang J. Targeting dendritic cells with exosomes: highly effective vaccine for induction of antitumor immunity. 2006, Submitted.
67. Hwang, I. & Sprent, J. Role of the actin cytoskeleton in T cell absorption and internalization of ligands from APC. J Immunol 166, 5099-107 (2001).
68. Dutton, R. W., Bradley, L. M. & Swain, S. L. T cell memory. Annu Rev Immunol 16, 201-23 (1998).
69. Liyanage, U. K. et al. Prevalence of regulatory T cells is increased in peripheral blood and tumor microenvironment of patients with pancreas or breast adenocarcinoma. J Immunol 169, 2756-61 (2002).
70. Kennedy, R. et al. Direct cross-priming by th lymphocytes generates memory cytotoxic T cell responses. J Immunol 174, 3967-77 (2005).
71. Kaech, S. M. et al. Selective expression of the interleukin 7 receptor identifies effector CD8 T cells that give rise to long-lived memory cells. Nat Immunol 4, 1191-8 (2003).
72. Kurts, C. et al. Constitutive class I-restricted exogenous presentation of self antigens in vivo. J Exp Med 184, 923-30 (1996).
73. Cozzo, C., Larkin, J., 3rd & Caton, A. J. Cutting edge: self-peptides drive the peripheral expansion of CD4+CD25+regulatory T cells. J Immunol 171, 5678-82 (2003).
74. Steinaa, L. et al. Linked foreign T-cell help activates self-reactive CTL and inhibits tumor growth. J Immunol 175, 329-34 (2005).
75. Wang, L. X., Kjaergaard, J., Cohen, P. A., Shu, S. & Plautz, G. E. Memory T cells originate from adoptively transferred effectors and reconstituting host cells after sequential lymphodepletion and adoptive immunotherapy. J Immunol 172, 3462-8 (2004).
76. Lanzavecchia, A. & Sallusto, F. Progressive differentiation and selection of the fittest in the immune response. Nat Rev Immunol 2, 982-7 (2002).
77. Geginat, J., Lanzavecchia, A. & Sallusto, F. Proliferation and differentiation potential of human CD8+ memory T-cell subsets in response to antigen or homeostatic cytokines. Blood 101, 4260-6 (2003).
78. Schwendemann, J., Choi, C., Schirrmacher, V. & Beckhove, P. Dynamic differentiation of activated human peripheral blood CD8+ and CD4+ effector memory T cells. J Immunol 175, 1433-9 (2005).
79. van Faassen, H. et al. Reducing the stimulation of CD8+ T cells during infection with intracellular bacteria promotes differentiation primarily into a central (CD62LhighCD44high) subset. J Immunol 174, 5341-50 (2005).
80. Bluestone, J. A. & Abbas, A. K. Natural versus adaptive regulatory T cells. Nat Rev Immunol 3, 253-7 (2003).
81. Curiel, T. J. et al. Specific recruitment of regulatory T cells in ovarian carcinoma fosters immune privilege and predicts reduced survival. Nat Med 10, 942-9 (2004).
82. Levings, M. K., Sangregorio, R. & Roncarolo, M. G. Human cd25(+)cd4(+) t regulatory cells suppress naïve and memory T cell proliferation and can be expanded in vitro without loss of function. J Exp Med 193, 1295-302 (2001).
83. Martin, B. et al. Suppression of CD4⁺ T lymphocyte effector functions by CD4+ CD25+ cells in vivo. J Immunol 172, 3391-8 (2004).
84. Nishikawa, H., Jager, E., Ritter, G., Old, L. J. & Gnjatic, S. CD4+ CD25+ regulatory T cells control the induction of antigen-specific CD4⁺ helper T cell responses in cancer patients. Blood 106, 1008-11 (2005).
85. Misra, N., Bayry, J., Lacroix-Desmazes, S., Kazatchkine, M. D. & Kaveri, S. V. Cutting edge: human CD4+ CD25+ T cells restrain the maturation and antigen-presenting function of dendritic cells. J Immunol 172, 4676-80 (2004).
86. Escudier, B. Dorval T, Chaput N, et al. Vaccination of metastatic melanoma patients with autologous dendritic cell (DC) derived-exosomes: results of the first phase I clinical trial. J Transl Med 3, 10 (2005).
87. Hwang, I., Shen, X. & Sprent, J. Direct stimulation of naïve T cells by membrane vesicles from antigen-presenting cells: distinct roles for CD54 and B7 molecules. Proc Natl Acad Sci USA 100, 6670-5 (2003).
88. Mor F, A W Lohse, N Karin, and I R Cohen. Clinical modeling of T cell vaccination against autoimmune diseases in rats. Selection of antigen-specific T cells using a mitogen. J Clin Invest 85, 1594-8 (1990).
89. Shapira 0, E Mor, T Reshef, R A Pfeffermann, and I R Cohen. Prolongation of survival of rat cardiac allografts by T cell vaccination. J Clin Invest 91, 388-90 (1993).
90. Kishimoto C, Hitoshi Takada, Yuji Hiraoka, Hiromichi Shinohara and Mikio Kitazawa. Protection against Murine Coxsackievirus B3 Myocarditis by T Cell Vaccination. J Mol Cell Cardiol 32, 2269-77 (2000).
91. Medaer R, Stinissen P, Truyen L, Raus J, Zhang J. Depletion of myelin-basic-protein autoreactive T cells by T-cell vaccination: pilot trial in multiple sclerosis. Lancet 346, 807-8 (1995).
92. Chen, Z., S. Dehm, K. Bonham, H. Kamencic, B. Juurlink, X. Zhang, J. R. Gordon, and J. Xiang. 2001. DNA array and biological characterization of the impact of the maturation status of mouse dendritic cells on their phenotype and antitumor vaccination efficacy. Cell Immunol 214:60.
93. Lyons, A. B., and C. R. Parish. 1994. Determination of lymphocyte division by flow cytometry. J Immunol Methods 171:131.
94. Hao, S., X. Bi, S. Xu, Y. Wei, X. Wu, X. Guo, S. Carlsen, and J. Xiang. 2004. Enhanced antitumor immunity derived from a novel vaccine of fusion hybrid between dendritic and engineered myeloma cells. Exp Oncol 26:300.
95. Clayton, A., J. Court, H. Navabi, M. Adams, M. D. Mason, J. A. Hobot, G. R. Newman, and B. Jasani. 2001. Analysis of antigen presenting cell derived exosomes, based on immuno-magnetic isolation and flow cytometry. J Immunol Methods 247:163.
96. Palucka, A. K., M. V. Dhodapkar, S. Paczesny, H. Ueno, J. Fay, and J. Banchereau. 2005. Boosting vaccinations with peptide-pulsed CD34+ progenitor-derived dendritic cells can expand long-lived melanoma peptide-specific CD8+ T cells in patients with metastatic melanoma. J Immunother 28:158.
97. Drickamer, K. 1999. C-type lectin-like domains. Curr Opin Struct Biol 9:585.
98. Denzer, K., M. J. Kleijmeer, H. F. Heijnen, W. Stoorvogel, and H. J. Geuze. 2000. Exosome: from internal vesicle of the multivesicular body to intercellular signaling device. J Cell Sci 113 Pt 19:3365.
99. Escola, J. M., M. J. Kleijmeer, W. Stoorvogel, J. M. Griffith, O. Yoshie, and H. J. Geuze. 1998. Selective enrichment of tetraspan proteins on the internal vesicles of multivesicular endosomes and on exosomes secreted by human B-lymphocytes. J Biol Chem 273:20121.
100. Akira, S., K. Takeda, and T. Kaisho. 2001. Toll-like receptors: critical proteins linking innate and acquired immunity. Nat Immunol 2:675.
101. Knight, S. C., S. Iqball, M. S. Roberts, S. Macatonia, and P. A. Bedford. 1998. Transfer of antigen between dendritic cells in the stimulation of primary T cell proliferation. Eur J Immunol 28:1636.
102. Cambi, A., K. Gijzen, J. M. de Vries, R. Torensma, B. Joosten, G. J. Adema, M. G. Netea, B. J. Kullberg, L. Romani, and C. G. Figdor. 2003. The C-type lectin DC-SIGN (CD209) is an antigen-uptake receptor for Candida albicans on dendritic cells. Eur J Immunol 33:532.
103. East, L., and C. M. Isacke. 2002. The mannose receptor family. Biochim Biophys Acta 1572:364.
104. Geijtenbeek, T. B., D. S. Kwon, R. Torensma, S. J. van Vliet, G. C. van Duijnhoven, J. Middel, I. L. Cornelissen, H. S. Nottet, V. N. KewalRamani, D. R. Littman, C. G. Figdor, and Y. van Kooyk. 2000. DC-SIGN, a dendritic cell-specific HIV-1-binding protein that enhances trans-infection of T cells. Cell 100:587.
105. Mahnke, K., M. Guo, S. Lee, H. Sepulveda, S. L. Swain, M. Nussenzweig, and R. M. Steinman. 2000. The dendritic cell receptor for endocytosis, DEC-205, can recycle and enhance antigen presentation via major histocompatibility complex class II-positive lysosomal compartments. J Cell Biol 151:673.
106. Engering, A., T. B. Geijtenbeek, S. J. van Vliet, M. Wijers, E. van Liempt, N. Demaurex, A. Lanzavecchia, J. Fransen, C. G. Figdor, V. Piguet, and Y. van Kooyk. 2002. The dendritic cell-specific adhesion receptor DC-SIGN internalizes antigen for presentation to T cells. J Immunol 168:2118.
107. Geijtenbeek, T. B., D. J. Krooshoop, D. A. Bleijs, S. J. van Vliet, G. C. van Duijnhoven, V. Grabovsky, R. Alon, C. G. Figdor, and Y. van Kooyk. 2000. DC-SIGN-ICAM-2 interaction mediates dendritic cell trafficking. Nat Immunol 1:353.
108. Vincent-Schneider, H., P. Stumptner-Cuvelette, D. Lankar, S. Pain, G. Raposo, P. Benaroch, and C. Bonnerot. 2002. Exosomes bearing HLA-DR1 molecules need dendritic cells to efficiently stimulate specific T cells. Int Immunol 14:713.

I claim:

1. A method of making a T helper-antigen presenting cell comprising contacting an exosome derived from a dendritic cell with a CD4⁺ T cell under conditions that allow absorption of the exosome on the CD4⁺ T cell.

2. The method according to claim 1, wherein the dendritic cell is bone marrow derived.

3. The method according to claim 1, wherein the CD4⁺ T cell is activated.

4. The method according to claim 1, wherein the CD4⁺ T cell is naïve.

5. The method according to claim 1, wherein the dendritic cell is exposed to an antigen prior to deriving the exosome from the dendritic cell.

6. An isolated T helper-antigen presenting cell made according to the method of claim 1.

7. A method of making a T helper-antigen presenting cell comprising contacting a CD4+ T cell with an activated dendritic cell under conditions that allow for transfer of molecules from the dendritic cell to the CD4+ T cells.

8. The method according to claim 7, wherein the molecules include antigen presentation machinery and/or costimulatory molecules.

9. The method according to claim 7, wherein the CD4+ T cell and the activated dendritic cell is contacted in the presence of IL-2, IL-12 and/or an anti-IL-4 antibody.

10. The method according to claim 7, wherein the activated dendritic cell is exposed to an antigen prior to contact with the CD4+ T cell.

11. An isolated T helper-antigen presenting cell made according to the method of claim 7.

12. A method of enhancing the immune response to treat or prevent a disease comprising administering an effective amount of a T helper-antigen presenting cell to an animal in need thereof.

13. The method according to claim 12, wherein the T helper-antigen presenting cell is administered in combination with other immune cells.

14. The method according to claim 13, wherein the other immune cells are dendritic cells, macrophages, B cells and/or T cells.

15. The method according to claim 12, wherein an immune adjuvant is used.

16. The method according to claim 12, wherein the disease is cancer, an immune disease or an infection.

17. The method according to claim 12, wherein cytotoxic T lymphocytes are activated.

18. A pharmaceutical composition for preventing or treating a disease comprising an effective amount of T helper-antigen presenting cells and a pharmaceutically acceptable carrier, diluent or excipient.

19. A method of making an exosome-absorbed dendritic cell comprising contacting an exosome derived from a first dendritic cell with a second dendritic cell under conditions that allow absorption of the exosome on the second dendritic cell.

20. The method according to claim 19, wherein the first dendritic cell is bone marrow derived.

21. The method according to claim 19, wherein the second dendritic cell is a mature dendritic cell.

22. The method according to claim 19, wherein the first dendritic cell is exposed to an antigen prior to deriving the exosome from the first dendritic cell.

23. An isolated exosome-absorbed dendritic cell made according to the method of claim 19.

24. A method of enhancing the immune response to treat or prevent a disease comprising administering an effective amount of an exosome-absorbed dendritic cell to an animal in need thereof.

25. The method according to claim 24, wherein the exosome-absorbed dendritic cell is administered in combination with other immune cells.

26. The method according to claim 25, wherein the other immune cells are dendritic cells, macrophages, B cells and/or T cells.

27. The method according to claim 24, wherein an immune adjuvant is used.

28. The method according to claim 24, wherein the disease is cancer, an immune disease or an infection.

29. The method according to claim 24, wherein cytotoxic T lymphocytes are activated.

30. A pharmaceutical composition for preventing or treating a disease comprising an effective amount of an exosome-absorbed dendritic cell and a pharmaceutically acceptable carrier, diluent or excipient.


SEQUENCE LISTING (3 sequences)

SEQ ID NO: 1 - protein, 8 aa, Artificial sequence
  OVAI synthetic peptide
SIINFEKL

SEQ ID NO: 2 - protein, 17 aa, Artificial sequence
  OVAII synthetic peptide
ISQAVHAAHAEINEAGR

SEQ ID NO: 3 - protein, 8 aa, Artificial sequence
  Mut1 synthetic peptide
FEQNTAQP